(19) United States
(12) Patent Application Publication
(10) Pub. No.: US 2015/0299120 A1
(43) Pub. Date: Oct. 22, 2015

(54) Spirohydantoin compounds and their use as selective androgen receptor modulators

(71) Applicant: NOVARTIS AG, Basel (CH)

(72) Inventors: Mark Gary BOCK, Boston, MA (US); Chetan PANDIT, Bangalore (IN); Sanjita SASMAL, Miyapur, Hyderabad (IN); Thomas ULLRICH, Basel (CH)

(21) Appl. No.: 14/382,073

(22) PCT Filed: Mar. 1, 2013
(86) PCT No.: PCT/IB2013/051651

§ 371 Date: Aug. 29, 2014

(30) Foreign Application Priority Data
Mar. 2, 2012 (IN) 609/DEL/2012

(51) Int. Cl. C07D 209/54; C07D 263/52; A61K 31/423; C07D 413/06; C07D 405/06; C07D 401/06; A61K 31/4439; C07D 491/107; A61K 31/4188; A61K 31/4184; A61K 31/422

(52) U.S. Cl. CPC C07D 209/54; A61K 31/4184; C07D 263/52; A61K 31/423; C07D 413/06; A61K 31/422; C07D 401/06; A61K 31/4439; C07D 491/107; A61K 31/4188; C07D 405/06

(57) ABSTRACT

The present invention relates to a compound of formula (I-1) in free form or in pharmaceutically acceptable salt form

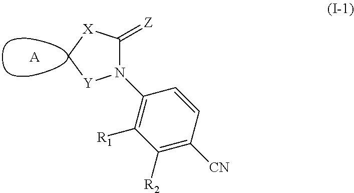

in which the substituents are as defined in the specification; to its preparation, to its use as a medicament and to medicaments comprising it. The present invention further provides a combination of pharmacologically active agents and a pharmaceutical composition.

The invention relates to spirohydantoin compounds, to their preparation, to their medical use as selective androgen receptor modulators and to medicaments, pharmaceutical compositions and combinations comprising them.

Selective androgen receptor modulators (SARMs) are ligands of the androgen receptor (AR) that have differential tissue regulation of AR. Selective androgen receptor modulators have been developed in the last decade as a new class of androgen receptor ligands analogous to androgenic drugs such as testosterone. Their improved selectivity over anabolic steroids suggests that this class of drugs could be developed for a number of therapeutic applications (Segal, S.; Narayanan, R.; Dalton J. T. Expert Opin. Investig. Drugs, 2006, 15(4), 377-387).

A number of disclosures such as WO97/19064, WO95/118794, US20110152348, WO2009055053, U.S. Pat. No. 5,750,553, U.S. Pat. No. 5,434,179, WO2011103202, WO2011029392, WO2010118354 and WO2007126765 disclose spiro compounds as anti-androgenics.

There is a continuing need to develop new modulators of the androgen receptor that are good drug candidates. SARMs would find wide application in conditions such as muscle wasting diseases, osteoporosis, sarcopenia, frailty, and cachexia (e.g. AIDS cachexia, cancer cachexia, COPD cachexia) in both men and women. In contrast to an androgen or to known AR antagonists, a desirable property of a SARM is that it would have an agonistic effect on the skeletal muscle and would be antagonistic or inactive in the prostate for example.

Compounds of the invention are selective for anabolic effect in e.g. muscle and bone tissue, and show beneficial effects in CNS while only having very limited androgenic effects in e.g. prostate and skin. The compounds of the invention show low affinity for other receptors. Particular compounds of the invention possess favourable pharmacokinetic properties, are non-toxic and demonstrate few side-effects. Furthermore, the ideal drug candidate will exist in a physical form that is stable, non-hygroscopic and easily formulated.

The compounds of the invention are selective androgen receptor modulators. They are therefore potentially useful in the treatment of a wide range of disorders or diseases, particularly muscle wasting diseases, osteoporosis, sarcopenia, frailty, and cachexia.

In a first aspect of the invention, there is therefore provided a compound of formula (I-1) in free form or in pharmaceutically acceptable salt form

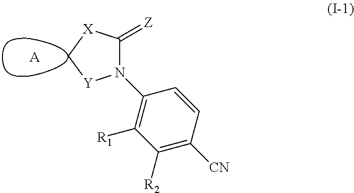

in which
X is O or N(R₈);
Y is CH₂, (C═NH), (C═O), (C═S) or CH(OR₉);
Z is O or S;
R₁ is C₁-C₃alkyl;
R₂ is halogen;
A is selected from:
   a 4-membered saturated ring which may contain one O atom, which ring is unsubstituted or substituted once or twice with R_{A}; or
   a 5-membered saturated or unsaturated non-aromatic ring which may contain one O atom, which ring is unsubstituted or substituted once or twice with R_{A};
R_{A} is, for each occurrence, independently selected from hydroxy, halogen, C₁-C₃alkyl, hydroxyC₁-C₃alkyl, or two R_{A} at the same carbon atom form an oxo group
R₈ is C₁-C₆alkyl optionally substituted with cyano, hydroxy-C₁-C₆alkyl, haloC₁-C₆alkyl, C₁-C₆alkoxy-C₁-C₆alkyl wherein the alkoxy portion is optionally substituted with cyano or halogen or
R₈ is —(CH₂)ₙ—B;
n is 1 or 2;
B is a 5- to 6-membered aromatic or non-aromatic ring which may comprise 1, 2, 3, or 4 heteroatoms selected from N, O or S, which ring is unsubstituted or substituted once or twice with R_{B};
R_{B} is, for each occurrence, independently selected from halo, cyano, C₁-C₆alkyl;
R₉ is hydrogen or C₁-C₃alkyl.

In a second aspect, the invention therefore provides a compound of formula (I) in free form or in pharmaceutically acceptable salt form

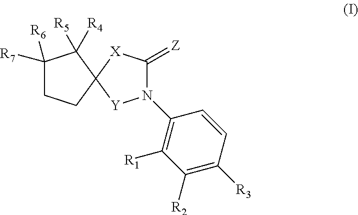

in which

### X is O or N(R₈);

### Y is CH₂, (C═O), (C═S) or CH(OR₉);

### Z is O or S;

R₁ is C₁-C₃alkyl;
5 R₂ is halogen;
R₃ is cyano;
R₄ and R₅ are independently selected from hydrogen, hydroxy or halogen; or R₄ and R₅ together form an oxo group;
R₆ and R₇ are independently selected from hydrogen, hydroxy, or halogen; or R₆ and R₇ together form an oxo group; or
R₄ and R₆ together form a bond and R₅ and R₇ are each hydrogen;
R₈ is C₁-C₃alkyl, C₁-C₆alkoxy-C₁-C₃alkyl, hydroxy-C₁-C₃alkyl;
R₉ is hydrogen or C₁-C₃alkyl.

Unless specified otherwise, the term “compounds of the present invention” refers to compounds of formula (I), (I-1), (Ia), (I-1a), (Ib), (I-1b), (Ic), (Id), (Ie), (If), (Ig), (Ih), (Ii) and (Ij), salts of the compounds, hydrates or solvates of the compounds and their salts, as well as all stereoisomers (including diastereoisomers and enantiomers), tautomers and isotopically labeled compounds (including deuterium substitutions), as well as inherently formed moieties (e.g., polymorphs, solvates and/or hydrates).

As used herein, the term “alkyl” refers to a fully saturated branched or unbranched hydrocarbon moiety having up to 6 carbon atoms. Unless otherwise provided, alkyl refers to hydrocarbon moieties having 1 to 6 carbon atoms, 1 to 4 carbon atoms or 1 to 3 carbon atoms. Representative examples of alkyl include, but are not limited to, methyl, ethyl, n-propyl, iso-propyl, tert-butyl and the like.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy and the like. Typically, alkoxy groups have 1-6, more preferably 1-4 carbons.

As used herein, the term “halogen” or “halo” refers to fluoro, chloro, bromo, and iodo. Typically, it refers to fluoro or chloro.

Typically, the term “selective androgen receptor modulators (SARMs)” includes compounds which are, for example, selective agonists, partial agonists, antagonists or partial antagonists of the androgen receptor.

Typically, the term “modulator” refers to a chemical compound with capacity to either enhance (e.g. “agonist” activity) or inhibit (e.g. “antagonist” activity) a functional property of biological activity or process (e.g. enzyme activity or receptor binding); such enhancement or inhibition may be contingent on the occurrence of a specific event, such as regulation of a signal transduction pathway, and/or may be manifest only in particular cell types.

Preferably, the SARMs of the present invention are selective agonists or partial agonists of the androgen receptor expressed in muscle and bone tissue.

Various embodiments of the invention are described herein. It will be recognized that features specified in each embodiment may be combined with other specified features to provide further embodiments.

In one embodiment, the invention provides a compound of formula (I) or (I-1) in free form or in pharmaceutically acceptable salt form as described above.

In one embodiment, the invention provides a compound of formula (I-1a) in free form or in pharmaceutically acceptable salt form

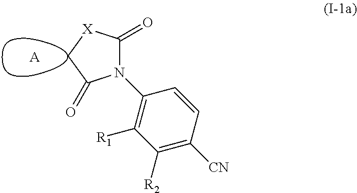

in which R₁, R₂, X, R₈, n, B, R_{B}, A and R_{A} are as defined in relation to the compound of formula (I-1).

In one embodiment, the invention provides a compound of formula (I-1b) in free form or in pharmaceutically acceptable salt form

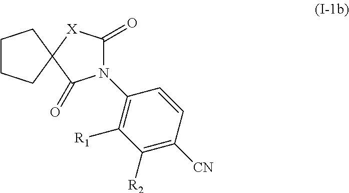

in which R₁, R₂, X, R₈, n, B, R_{B} are as defined in relation to the compound of formula (I-1).

In one embodiment, the invention provides a compound of formula (Ia) in free form or in pharmaceutically acceptable salt form

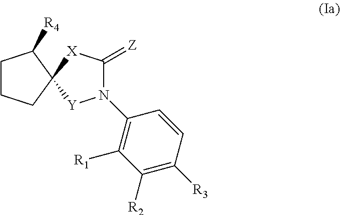

in which X, Y, Z, R₁, R₂, R₃, R₄, R₈ and R₉ are as defined in relation to the compound of formula (I).

In one embodiment, the invention provides a compound of formula (Ia) in free form or in pharmaceutically acceptable salt form where Y is (C═O), Z is O, R₁, R₂, R₃, R₄, X are as defined in relation to the compound of formula (I) and R₄ is not hydrogen.

In one embodiment, the invention provides a compound of formula (Ib) in free form or in pharmaceutically acceptable salt form

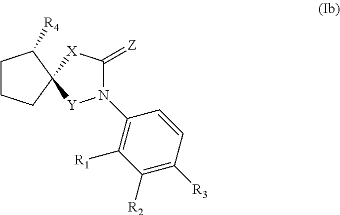

in which X, Y, Z, R₁, R₂, R₃, R₄, R₈ and R⁹ are as defined in relation to the compound of formula (I).

In one embodiment, the invention provides a compound of formula (Ib) in free form or in pharmaceutically acceptable salt form where Y is (C═O), Z is O, R₁, R₂, R₃, R₄, X are as defined in relation to the compound of formula (I) and R₄ is not hydrogen.

In one embodiment, the invention provides a compound of formula (Ic) in free form or in pharmaceutically acceptable salt form

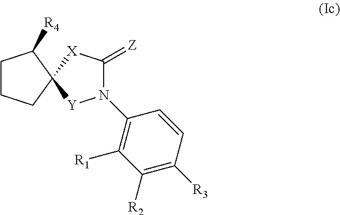

in which X, Y, Z, R₁, R₂, R₃, R₄, R₈ and R₉ are as defined in relation to the compound of formula (I).

In one embodiment, the invention provides a compound of formula (Ic) in free form or in pharmaceutically acceptable salt form where Y is (C═O), Z is O, R₁, R₂, R₃, R₄, X are as defined in relation to the compound of formula (I) and R₄ is not hydrogen.

In one embodiment, the invention provides a compound of formula (Id) in free form or in pharmaceutically acceptable salt form

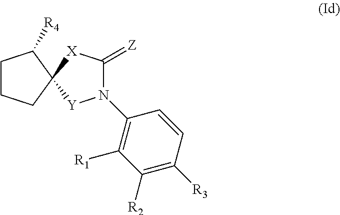

in which X, Y, Z, R₁, R₂, R₃, R₄, R₈ and R₉ are as defined in relation to the compound of formula (I).

In one embodiment, the invention provides a compound of formula (Id) in free form or in pharmaceutically acceptable salt form where Y is (C═O), Z is O, R₁, R₂, R₃, R₄, X are as defined in relation to the compound of formula (I) and R₄ is not hydrogen.

In one embodiment, the invention provides a compound of formula (Ie) in free form or in pharmaceutically acceptable salt form

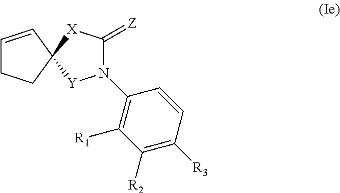

in which X, Y, Z, R₁, R₂, R₃, R₈ and R₉ are as defined in relation to the compound of formula (I).

In one embodiment, the invention provides a compound of formula (Ie) in free form or in pharmaceutically acceptable salt form where Y is (C═O), Z is O, R₁, R₂, R₃, X are as defined in relation to the compound of formula (I).

In one embodiment, the invention provides a compound of formula (If) in free form or in pharmaceutically acceptable salt form

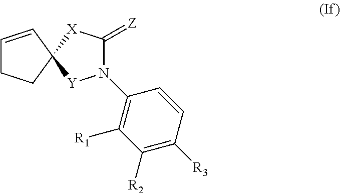

in which X, Y, Z, R₁, R₂, R₃, R₈ and R₉ are as defined in relation to the compound of formula (I).

In one embodiment, the invention provides a compound of formula (If) in free form or in pharmaceutically acceptable salt form where Y is (C═O), Z is O, R₁, R₂, R₃, X are as defined in relation to the compound of formula (I).

In one embodiment, the invention provides a compound of formula (Ig) in free form or in pharmaceutically acceptable salt form

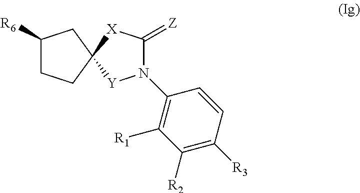

in which X, Y, Z, R₁, R₂, R₃, R₆, R₈ and R₉ are as defined in relation to the compound of formula (I).

In one embodiment, the invention provides a compound of formula (Ig) in free form or in pharmaceutically acceptable salt form where Y is (C═O), Z is O, R₁, R₂, R₃, R₆, X are as defined in relation to the compound of formula (I) and R₆ is not hydrogen.

In one embodiment, the invention provides a compound of formula (Ih) in free form or in pharmaceutically acceptable salt form

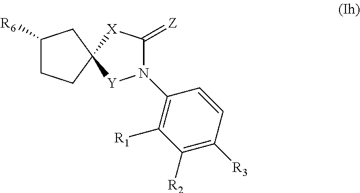

in which X, Y, Z, R₁, R₂, R₃, R₆, R₈ and R₉ are as defined in relation to the compound of formula (I).

In one embodiment, the invention provides a compound of formula (Ih) in free form or in pharmaceutically acceptable salt form where Y is (C═O), Z is O, R₁, R₂, R₃, R₆, X are as defined in relation to the compound of formula (I) and R₆ is not hydrogen.

In one embodiment, the invention provides a compound of formula (Ii) in free form or in pharmaceutically acceptable salt form

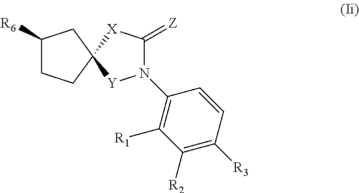

in which X, Y, Z, R₁, R₂, R₃, R₆, R₈ and R₉ are as defined in relation to the compound of formula (I).

In one embodiment, the invention provides a compound of formula (Ii) in free form or in pharmaceutically acceptable salt form where Y is (C═O), Z is O, R₁, R₂, R₃, R₆, X are as defined in relation to the compound of formula (I) and R₆ is not hydrogen.

In one embodiment, the invention provides a compound of formula (Ij) in free form or in pharmaceutically acceptable salt form

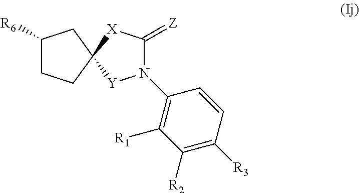

in which X, Y, Z, R₁, R₂, R₃, R₆, R₈ and R₉ are as defined in relation to the compound of formula (I).

In one embodiment, the invention provides a compound of formula (Ij) in free form or in pharmaceutically acceptable salt form where Y is (C═O), Z is O, R₁, R₂, R₃, R₆, X are as defined in relation to the compound of formula (I) and R₆ is not hydrogen.

In one embodiment, the invention provides a compound of formula (I-1b) in free form or in pharmaceutically acceptable salt form wherein

### X is N(R₈) and

R₈ is C₁-C₃alkyl, C₁-C₆alkoxy-C₁-C₃alkyl, hydroxy-C₁-C₃alkyl.

In one embodiment, the invention provides a compound of formula (I-1b) in free form or in pharmaceutically acceptable salt form wherein

### X is N(R₈)

### R₈ is —(CH₂)—B;

B is a 5-membered aromatic ring comprising 1 or 2 heteroatoms selected from N, O or S, which ring is unsubstituted or substituted once or twice with R_{B};
R_{B} is C₁-C₆alkyl.

In one embodiment, the invention provides a compound of formula (I-1b) in free form or in pharmaceutically acceptable salt form wherein

### X is N(R₈)

### R₈ is —(CH₂)—B;

B is a 6-membered aromatic ring which may comprise one N atom, which ring is unsubstituted or substituted once or twice with R_{B};
R_{B} is, for each occurrence, selected from halo, cyano or C₁-C₆alkyl.

In one embodiment, the invention provides a compound of formula (I) in free form or in pharmaceutically acceptable salt form wherein

### X is N(R₈);

### Y is (C═O);

### Z is O;

R₁ is C₁-C₃alkyl;
R₂ is halogen;
R₃ is cyano;
R₄ and R₅ are independently selected from hydrogen, hydroxy or halogen; or R₄ and R₅ together form an oxo group;
R₆ and R₇ are independently selected from hydrogen, hydroxy, or halogen; or R₆ and R₇ together form an oxo group; or
R₄ and R₆ form a bond and R₅ and R₇ are hydrogen;
R₈ is C₁-C₃alkyl, C₁-C₆alkoxy-C₁-C₃alkyl, hydroxy-C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄ and R₅ are independently selected from hydrogen, hydroxy or halogen; or R₄ and R₅ together form an oxo group;
R₆ and R₇ are independently selected from hydrogen, hydroxy, or halogen; or R₆ and R₇ together form an oxo group; or
R₄ and R₆ form a bond and R₅ and R₇ are hydrogen;
R₈ is C₁-C₃alkyl, C₁-C₆alkoxy-C₁-C₃alkyl, hydroxy-C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄ is selected from hydroxy or halogen;
R₅, R₆ and R₇ are hydrogen;
R₈ is C₁-C₃alkyl, C₁-C₆alkoxy-C₁-C₃alkyl, hydroxy-C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄ is selected from hydroxy or halogen;
R₅, R₆ and R₇ are hydrogen;
R₈ is C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄ and R₆ form a bond and R₅ and R₇ are hydrogen;
R₈ is C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄, R₅, R₆ and R₇ are hydrogen;
R₈ is C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄, R₅ and R₇ are hydrogen;
R₆ is selected from hydroxy or halogen;
R₈ is C₁-C₃alkyl.

In one embodiment, the invention provides a compound of formula (I) in free form or in pharmaceutically acceptable salt form wherein

### X is N(R₈);

### Y is (C═O);

### Z is O;

R₁ is methyl;
R₂ is chloro;
R₃ is cyano;
R₄ and R₅ are hydrogen;
R₆ and R₇ are halogen;
R₈ is C₁-C₃alkyl.

In certain embodiments, the invention relates to a compound of formula (I), (I-1), (Ia), (I-1a), (Ib), (I-1b), (Ic), (Id), (Ie), (If), (Ig), (Ih), (Ii) and (Ij) in free form or in pharmaceutically acceptable salt form, in which, where appropriate:
(1) R₁ is methyl;
(2) R₁ is ethyl;
(3) R₁ is n-propyl or isopropyl;
(4) R₂ is chloro;
(5) R₂ is fluoro;
(6) R₄ is hydrogen;
(7) R₄ is hydroxy;
(8) R₄ is halogen;
(9) R₄ is fluoro;
(10) R₄ is chloro;
(11) R₅ is hydrogen;
(12) R₅ is hydroxy;
(13) R₅ is halogen;
(14) R₅ is fluoro;
(15) R₅ is chloro;
(16) R₄ and R₅ together form oxo;
(17) R₆ is hydrogen;
(18) R₆ is hydroxy;
(19) R₆ is halogen;
(20) R₆ is fluoro;
(21) R₆ is chloro;
(22) R₇ is hydrogen;
(23) R₇ is hydroxy;
(24) R₇ is halogen;
(25) R₇ is fluoro;
(26) R₇ is chloro;
(27) R₆ and R₇ together form oxo;
(28) R₄ and R₆ together form a bond and R₅ and R₇ are each hydrogen;
(29) R₈ is C₁-C₃alkyl;
(30) R₈ is methyl;
(31) R₈ is ethyl;
(32) R₈ is propyl;
(33) R₈ is cyanoC₁-C₆alkyl;
(34) R₈ is 3-cyanopropyl
(35) R₈ is 4-cyanobutyl
(36) R₈ is 5-cyanopentyl
(37) R₈ is C₁-C₆alkoxy-C₁-C₃alkyl;
(38) R₈ is methoxymethyl, methoxyethyl or methoxypropyl;
(39) R₈ is ethoxymethyl, ethoxyethyl, or ethoxypropyl;
(40) R₈ is isopropoxymethyl, isopropoxyethyl or isopropoxypropyl;
(41) R₈ is C₁-C₆alkoxy-C₁-C₆alkyl where the alkoxy portion is substituted with cyano or halogen;
(42) R₈ is cyanoethoxyethyl;
(43) R₈ is 2-fluoroethoxyethyl;
(44) R₈ is hydroxyC₁-C₃alkyl;
(45) R₈ is hydroxymethyl, hydroxyethyl or hydroxypropyl;
(46) R₉ is hydrogen;
(47) R₉ is C₁-C₃alkyl;
(48) R₉ is methyl;
(49) R₉ is ethyl;
(50) R₉ is propyl;
(51) X is N(R₈);
(52) X is O;
(53) Y is (C═O);
(54) Z is O;
(55) A is a 4-membered saturated carbocyclic ring;
(56) A is a 4-membered saturated ring comprising one O atom;
(57) A is a 5-membered unsubstituted saturated carbocyclic ring;
(58) A is a 5-membered unsubstituted saturated ring comprising one O atom;
(59) A is a 5-membered saturated carbocyclic ring substituted once or twice with R_{A};
(60) A is a 5-membered saturated carbocyclic ring substituted once with methyl
(61) B is a 5-membered heterocyclic aromatic ring comprising 1 or 2 heteroatoms selected from N, O or S;
(62) B is oxazolyl or isoxazolyl;
(63) B is oxazolyl or isoxazolyl substituted once or twice with C₁-C₆alkyl;
(64) B is a 6-membered aromatic ring which may comprise one N atom;
(65) B is pyridyl;
(66) B is pyridyl substituted once or twice with C₁-C₆alkyl;
(67) B is phenyl.

The skilled person would understand that the embodiments (1) to (67) may be used independently, collectively or in any combination or sub-combination to limit the scope of the invention as described hereinbefore in relation to compounds of formula (I), (Ia), (Ib), (Ic), (Id), (Ie), (If), (Ig), (Ih), (Ii) and (Ij) as appropriate.

In one embodiment, the invention provides a compound which is selected from
2-chloro-4-(6-hydroxy-1-methyl-2,4-dioxo-1,3-diazaspiro[4.4]nonan-3-yl)-3-methylbenzonitrile;
2-chloro-3-methyl-4-(1-methyl-2,4,6-trioxo-1,3-diazaspiro[4.4]nonan-3-yl)benzonitrile;
2-chloro-4-(4,6-dihydroxy-1-methyl-2-oxo-1,3-diazaspiro[4.4]nonan-3-yl)-3-methylbenzonitrile;
2-chloro-4-(1-ethyl-6-hydroxy-2,4-dioxo-1,3-diazaspiro[4.4]nonan-3-yl)-3-methylbenzonitrile;
2-chloro-4-(6-fluoro-1-methyl-2,4-dioxo-1,3-diazaspiro[4.4]nonan-3-yl)-3-methylbenzonitrile;
2-chloro-3-methyl-4-(1-methyl-2,4-dioxo-1,3-diazaspiro[4.4]non-6-en-3-yl)benzonitrile;
2-chloro-4-(6-hydroxy-1-methyl-2-oxo-1,3-diazaspiro[4.4]nonan-3-yl)-3-methylbenzonitrile;
2-chloro-3-methyl-4-(1-methyl-2,4-dioxo-1,3-diazaspiro[4.4]nonan-3-yl)benzonitrile;
2-chloro-4-(1-(2-methoxyethyl)-2,4-dioxo-1,3-diazaspiro[4.4]nonan-3-yl)-3-methylbenzonitrile;
2-chloro-4-(1-ethyl-2,4-dioxo-1,3-diazaspiro[4.4]nonan-3-yl)-3-methylbenzonitrile;
2-chloro-4-(6-hydroxy-2,4-dioxo-1-oxa-3-azaspiro[4.4]nonan-3-yl)-3-methylbenzonitrile;
2-chloro-4-(6-hydroxy-4-methoxy-2-oxo-1-oxa-3-azaspiro[4.4]nonan-3-yl)-3-methylbenzonitrile;
2-chloro-3-methyl-4-(2-oxo-1-oxa-3-azaspiro[4.4]non-6-en-3-yl)benzonitrile;
2-chloro-4-(7-hydroxy-1-methyl-2,4-dioxo-1,3-diazaspiro[4.4]nonan-3-yl)-3-methylbenzonitrile;
2-chloro-4-(7-fluoro-1-methyl-2,4-dioxo-1,3-diazaspiro[4.4]nonan-3-yl)-3-methylbenzonitrile;
2-chloro-3-methyl-4-(1-methyl-2,4,7-trioxo-1,3-diazaspiro[4.4]nonan-3-yl)benzonitrile;
2-chloro-4-(7,7-difluoro-1-methyl-2,4-dioxo-1,3-diazaspiro[4.4]nonan-3-yl)-3-methylbenzonitrile;
2-chloro-4-(6-(hydroxymethyl)-1-methyl-2,4-dioxo-1,3-diazaspiro[4.4]nonan-3-yl)-3-methylbenzonitrile;
2-chloro-4-(1,6-dimethyl-2,4-dioxo-1,3-diazaspiro[4.4]nonan-3-yl)-3-methylbenzonitrile;
2-chloro-4-(4-imino-1-methyl-2-oxo-1,3-diazaspiro[4.4]nonan-3-yl)-3-methylbenzonitrile;
2-chloro-4-(1-(4-cyanobenzyl)-2,4-dioxo-1,3-diazaspiro[4.4]nonan-3-yl)-3-methylbenzonitrile;
2-chloro-3-methyl-4-(1-((5-methyl isoxazol-3-yl)methyl)-2,4-dioxo-1,3-diazaspiro[4.4]nonan-3-yl)benzonitrile;
2-chloro-4-(2,4-dioxo-1-(2-(pyridin-4-yl)ethyl)-1,3-diazaspiro[4.4]nonan-3-yl)-3-methylbenzonitrile;
2-chloro-4-(1-((3,5-dimethylisoxazol-4-yl)methyl)-2,4-dioxo-1,3-diazaspiro[4.4]nonan-3-yl)-3-methylbenzonitrile;
2-chloro-4-(2,4-dioxo-1-(pyridin-2-ylmethyl)-1,3-diazaspiro[4.4]nonan-3-yl)-3-methylbenzonitrile;
2-chloro-4-(2,4-dioxo-1-(pyridin-4-ylmethyl)-1,3-diazaspiro[4.4]nonan-3-yl)-3-methylbenzonitrile;
2-chloro-3-methyl-4-(1-((6-methyl pyridin-3-yl)methyl)-2,4-dioxo-1,3-diazaspiro[4.4]nonan-3-yl)benzonitrile;
2-chloro-3-methyl-4-(1-((5-methyloxazol-2-yl)methyl)-2,4-dioxo-1,3-diazaspiro[4.4]nonan-3-yl)benzonitrile;
2-chloro-4-(1-((5-(hydroxymethyl)oxazol-2-yl)methyl)-2,4-dioxo-1,3-diazaspiro[4.4]nonan-3-yl)-3-methylbenzonitrile;
2-chloro-3-methyl-4-(1-(oxazol-5-ylmethyl)-2,4-dioxo-1,3-diazaspiro[4.4]nonan-3-yl)benzonitrile;
2-chloro-3-methyl-4-(1-((2-methyloxazol-5-yl)methyl)-2,4-dioxo-1,3-diazaspiro[4.4]nonan-3-yl)benzonitrile;
2-chloro-4-(1-(2-fluoroethyl)-2,4-dioxo-1,3-diazaspiro[4.4]nonan-3-yl)-3-methylbenzonitrile;
2-chloro-4-(1-(5-cyanopentyl)-2,4-dioxo-1,3-diazaspiro[4.4]nonan-3-yl)-3-methylbenzonitrile;
2-chloro-4-(1-(2-(2-fluoroethoxy)ethyl)-2,4-dioxo-1,3-diazaspiro[4.4]nonan-3-yl)-3-methylbenzonitrile;
2-chloro-4-(1-(2-(cyanomethoxy)ethyl)-2,4-dioxo-1,3-diazaspiro[4.4]nonan-3-yl)-3-methylbenzonitrile;
2-chloro-4-(1-(3-cyanopropyl)-2,4-dioxo-1,3-diazaspiro[4.4]nonan-3-yl)-3-methylbenzonitrile;
2-chloro-4-(1-isobutyl-2,4-dioxo-1,3-diazaspiro[4.4]nonan-3-yl)-3-methylbenzonitrile;
2-chloro-4-(1-(4-cyanobutyl)-2,4-dioxo-1,3-diazaspiro[4.4]nonan-3-yl)-3-methylbenzonitrile;
2-chloro-4-(1-(2-hydroxyethyl)-2,4-dioxo-1,3-diazaspiro[4.4]nonan-3-yl)-3-methylbenzonitrile;
2-chloro-4-(1-(2-cyanoethyl)-2,4-dioxo-1,3-diazaspiro[4.4]nonan-3-yl)-3-methylbenzonitrile;
2-chloro-4-(1-(3-fluoropropyl)-2,4-dioxo-1,3-diazaspiro[4.4]nonan-3-yl)-3-methylbenzonitrile;
2-chloro-3-methyl-4-(1-methyl-2,4-dioxo-7-oxa-1,3-diazaspiro[4.4]nonan-3-yl)benzonitrile;
2-chloro-4-(2,4-dioxo-1-((tetrahydrofuran-3-yl)methyl)-1,3-diazaspiro[4.4]nonan-3-yl)-3-methylbenzonitrile and
2-chloro-3-methyl-4-(5-methyl-6,8-dioxo-5,7-diazaspiro[3.4]octan-7-yl)benzonitrile
   in free form or in pharmaceutically acceptable salt form.

In one embodiment, the invention provides a compound which is selected from
2-chloro-4-((5R,6S)-6-hydroxy-1-methyl-2,4-dioxo-1,3-diazaspiro[4.4]nonan-3-yl)-3-methylbenzonitrile;
2-chloro-4-((5S,6R)-6-hydroxy-1-methyl-2,4-dioxo-1,3-diazaspiro[4.4]nonan-3-yl)-3-methylbenzonitrile;
2-chloro-4-((5R,6R)-6-hydroxy-1-methyl-2,4-dioxo-1,3-diazaspiro[4.4]nonan-3-yl)-3-methylbenzonitrile;
2-chloro-4-((5S,6S)-6-hydroxy-1-methyl-2,4-dioxo-1,3-diazaspiro[4.4]nonan-3-yl)-3-methylbenzonitrile;
(R)-2-chloro-3-methyl-4-(1-methyl-2,4,6-trioxo-1,3-diazaspiro[4.4]nonan-3-yl)benzonitrile;
(S)-2-chloro-3-methyl-4-(1-methyl-2,4,6-trioxo-1,3-diazaspiro[4.4]nonan-3-yl)benzonitrile;
2-chloro-4-((4R,5R,6R)-4,6-dihydroxy-1-methyl-2-oxo-1,3-diazaspiro[4.4]nonan-3-yl)-3-methylbenzonitrile;
2-chloro-4-((4R,5R,6S)-4,6-dihydroxy-1-methyl-2-oxo-1,3-diazaspiro[4.4]nonan-3-yl)-3-methylbenzonitrile;
2-chloro-4-((4R,5S,6S)-4,6-dihydroxy-1-methyl-2-oxo-1,3-diazaspiro[4.4]nonan-3-yl)-3-methylbenzonitrile;
2-chloro-4-((4R,5S,6R)-4,6-dihydroxy-1-methyl-2-oxo-1,3-diazaspiro[4.4]nonan-3-yl)-3-methylbenzonitrile;
2-chloro-4-((4S,5R,6R)-4,6-dihydroxy-1-methyl-2-oxo-1,3-diazaspiro[4.4]nonan-3-yl)-3-methylbenzonitrile;
2-chloro-4-((4S,5R,6S)-4,6-dihydroxy-1-methyl-2-oxo-1,3-diazaspiro[4.4]nonan-3-yl)-3-methylbenzonitrile;
2-chloro-4-((4S,5S,6S)-4,6-dihydroxy-1-methyl-2-oxo-1,3-diazaspiro[4.4]nonan-3-yl)-3-methylbenzonitrile;
2-chloro-4-((4S,5S,6R)-4,6-dihydroxy-1-methyl-2-oxo-1,3-diazaspiro[4.4]nonan-3-yl)-3-methylbenzonitrile;
2-chloro-4-((5S,6R)-1-ethyl-6-hydroxy-2,4-dioxo-1,3-diazaspiro[4.4]nonan-3-yl)-3-methylbenzonitrile;
2-chloro-4-((5S,6S)-1-ethyl-6-hydroxy-2,4-dioxo-1,3-diazaspiro[4.4]nonan-3-yl)-3-methylbenzonitrile;
2-chloro-4-((5R,6S)-1-ethyl-6-hydroxy-2,4-dioxo-1,3-diazaspiro[4.4]nonan-3-yl)-3-methylbenzonitrile;
2-chloro-4-((5R,6R)-1-ethyl-6-hydroxy-2,4-dioxo-1,3-diazaspiro[4.4]nonan-3-yl)-3-methylbenzonitrile;
2-chloro-4-((5S,6S)-6-fluoro-1-methyl-2,4-dioxo-1,3-diazaspiro[4.4]nonan-3-yl)-3-methylbenzonitrile;
2-chloro-4-((5S,6R)-6-fluoro-1-methyl-2,4-dioxo-1,3-diazaspiro[4.4]nonan-3-yl)-3-methylbenzonitrile;
2-chloro-4-((5R,6S)-6-fluoro-1-methyl-2,4-dioxo-1,3-diazaspiro[4.4]nonan-3-yl)-3-methylbenzonitrile;
2-chloro-4-((5R,6R)-6-fluoro-1-methyl-2,4-dioxo-1,3-diazaspiro[4.4]nonan-3-yl)-3-methylbenzonitrile;
(S)-2-chloro-3-methyl-4-(1-methyl-2,4-dioxo-1,3-diazaspiro[4.4]non-6-en-3-yl)benzonitrile;
(R)-2-chloro-3-methyl-4-(1-methyl-2,4-dioxo-1,3-diazaspiro[4.4]non-6-en-3-yl)benzonitrile;
2-chloro-4-((5S,6R)-6-hydroxy-1-methyl-2-oxo-1,3-diazaspiro[4.4]nonan-3-yl)-3-methylbenzonitrile;
2-chloro-4-((5R,6R)-6-hydroxy-1-methyl-2-oxo-1,3-diazaspiro[4.4]nonan-3-yl)-3-methylbenzonitrile;
2-chloro-4-((5R,6S)-6-hydroxy-1-methyl-2-oxo-1,3-diazaspiro[4.4]nonan-3-yl)-3-methylbenzonitrile;
2-chloro-4-((5S,6S)-6-hydroxy-1-methyl-2-oxo-1,3-diazaspiro[4.4]nonan-3-yl)-3-methylbenzonitrile;
2-chloro-4-((5S,6R)-6-hydroxy-2,4-dioxo-1-oxa-3-azaspiro[4.4]nonan-3-yl)-3-methylbenzonitrile;
2-chloro-4-((5S,6S)-6-hydroxy-2,4-dioxo-1-oxa-3-azaspiro[4.4]nonan-3-yl)-3-methylbenzonitrile;
2-chloro-4-((5R,6R)-6-hydroxy-2,4-dioxo-1-oxa-3-azaspiro[4.4]nonan-3-yl)-3-methylbenzonitrile;
2-chloro-4-((5R,6S)-6-hydroxy-2,4-dioxo-1-oxa-3-azaspiro[4.4]nonan-3-yl)-3-methylbenzonitrile;
2-chloro-4-((4R,5R,6R)-6-hydroxy-4-methoxy-2-oxo-1-oxa-3-azaspiro[4.4]nonan-3-yl)-3-methylbenzonitrile;
2-chloro-4-((4R,5R,6S)-6-hydroxy-4-methoxy-2-oxo-1-oxa-3-azaspiro[4.4]nonan-3-yl)-3-methylbenzonitrile;
2-chloro-4-((4R,5S,6S)-6-hydroxy-4-methoxy-2-oxo-1-oxa-3-azaspiro[4.4]nonan-3-yl)-3-methylbenzonitrile;
2-chloro-4-((4R,5S,6R)-6-hydroxy-4-methoxy-2-oxo-1-oxa-3-azaspiro[4.4]nonan-3-yl)-3-methylbenzonitrile;
2-chloro-4-((4S,5R,6R)-6-hydroxy-4-methoxy-2-oxo-1-oxa-3-azaspiro[4.4]nonan-3-yl)-3-methylbenzonitrile;
2-chloro-4-((4S,5R,6S)-6-hydroxy-4-methoxy-2-oxo-1-oxa-3-azaspiro[4.4]nonan-3-yl)-3-methylbenzonitrile;
2-chloro-4-((4S,5S,6R)-6-hydroxy-4-methoxy-2-oxo-1-oxa-3-azaspiro[4.4]nonan-3-yl)-3-methylbenzonitrile;
2-chloro-4-((4S,5S,6S)-6-hydroxy-4-methoxy-2-oxo-1-oxa-3-azaspiro[4.4]nonan-3-yl)-3-methylbenzonitrile;
(S)-2-chloro-3-methyl-4-(2-oxo-1-oxa-3-azaspiro[4.4]non-6-en-3-yl)benzonitrile;
(R)-2-chloro-3-methyl-4-(2-oxo-1-oxa-3-azaspiro[4.4]non-6-en-3-yl)benzonitrile;
2-chloro-4-((5R,7R)-7-hydroxy-1-methyl-2,4-dioxo-1,3-diazaspiro[4.4]nonan-3-yl)-3-methylbenzonitrile;
2-chloro-4-((5R,7S)-7-hydroxy-1-methyl-2,4-dioxo-1,3-diazaspiro[4.4]nonan-3-yl)-3-methylbenzonitrile;
2-chloro-4-((5S,7R)-7-hydroxy-1-methyl-2,4-dioxo-1,3-diazaspiro[4.4]nonan-3-yl)-3-methylbenzonitrile;
2-chloro-4-((5S,7S)-7-hydroxy-1-methyl-2,4-dioxo-1,3-diazaspiro[4.4]nonan-3-yl)-3-methylbenzonitrile;
2-chloro-4-((5R,7R)-7-fluoro-1-methyl-2,4-dioxo-1,3-diazaspiro[4.4]nonan-3-yl)-3-methylbenzonitrile;
2-chloro-4-((5R,7S)-7-fluoro-1-methyl-2,4-dioxo-1,3-diazaspiro[4.4]nonan-3-yl)-3-methylbenzonitrile;
2-chloro-4-((5S,7R)-7-fluoro-1-methyl-2,4-dioxo-1,3-diazaspiro[4.4]nonan-3-yl)-3-methylbenzonitrile;
2-chloro-4-((5S,7S)-7-fluoro-1-methyl-2,4-dioxo-1,3-diazaspiro[4.4]nonan-3-yl)-3-methylbenzonitrile;
(R)-2-chloro-3-methyl-4-(1-methyl-2,4,7-trioxo-1,3-diazaspiro[4.4]nonan-3-yl)benzonitrile;
(S)-2-chloro-3-methyl-4-(1-methyl-2,4,7-trioxo-1,3-diazaspiro[4.4]nonan-3-yl)benzonitrile;
(R)-2-chloro-4-(7,7-difluoro-1-methyl-2,4-dioxo-1,3-diazaspiro[4.4]nonan-3-yl)-3-methylbenzonitrile;
(S)-2-chloro-4-(7,7-difluoro-1-methyl-2,4-dioxo-1,3-diazaspiro[4.4]nonan-3-yl)-3-methylbenzonitrile
   in free form or in pharmaceutically acceptable salt form.

Preferably, a compound of the invention is not 2-chloro-4-(4-hydroxy-1-methyl-2-oxo-1,3-diazaspiro[4.4]nonan-3-yl)-3-methylbenzonitrile, 2-chloro-4-(4-methoxy-1-methyl-2-oxo-1,3-diazaspiro[4.4]nonan-3-yl)-3-methylbenzonitrile, 2-chloro-4-(6-hydroxy-2-oxo-1-oxa-3-azaspiro[4.4]nonan-3-yl)-3-methylbenzonitrile, 2-chloro-4-(6-fluoro-2-oxo-1-oxa-3-azaspiro[4.4]nonan-3-yl)-3-methylbenzonitrile, 2-chloro-4-(6-fluoro-1-methyl-2-oxo-1,3-diazaspiro[4.4]nonan-3-yl)-3-methylbenzonitrile, 2-chloro-4-(2,4-dioxo-1-(2-(tetrahydro-2H-pyran-4-yl)ethyl)-1,3-diazaspiro[4.4]nonan-3-yl)-3-methylbenzonitrile, 2-chloro-3-methyl-4-(1-(2-morpholinoethyl)-2,4-dioxo-1,3-diazaspiro[4.4]nonan-3-yl)benzonitrile, 2-chloro-4-(1-(2-ethoxyethyl)-2,4-dioxo-1,3-diazaspiro[4.4]nonan-3-yl)-3-methylbenzonitrile, 2-chloro-4-(1-(2-isobutoxyethyl)-2,4-dioxo-1,3-diazaspiro[4.4]nonan-3-yl)-3-methylbenzonitrile, or 2-chloro-4-(1-(2-isopropoxyethyl)-2,4-dioxo-1,3-diazaspiro[4.4]nonan-3-yl)-3-methylbenzonitrile.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

On account of one or more than one asymmetrical carbon atom, which may be present in a compound of the formula (I) or (I-1), a corresponding compound of the formula (I) or (I-1) may exist in pure optically active form or in the form of a mixture of optical isomers, e.g. in the form of a racemic mixture. All of such pure optical isomers and all of their mixtures, including the racemic mixtures, are part of the present invention.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, glycolate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention, if formed, can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, EtOD or CH₃CO₂D.

Compounds of the invention, i.e. compounds of formula (I), (I-1), (Ia), (I-1a), (Ib), (I-1b),-(Ic), (Id), (Ie), (If), (Ig), (Ih), (Ii) and (Ij) that contain groups capable of acting as donors and/or acceptors for hydrogen bonds may be capable of forming co-crystals with suitable co-crystal formers. These co-crystals may be prepared from compounds of formula (I), (Ia), (Ib), (Ic), (Id), (Ie), (If), (Ig), (Ih), (Ii) and (Ij) by known co-crystal forming procedures. Such procedures include grinding, heating, co-subliming, co-melting, or contacting in solution compounds of formula (I), (Ia), (Ib), (Ic), (Id), (Ie), (If), (Ig), (Ih), (Ii) and (Ij) with the co-crystal former under crystallization conditions and isolating co-crystals thereby formed. Suitable co-crystal formers include those described in WO 2004/078163. Hence the invention further provides co-crystals comprising a compound of formula (I), (Ia), (Ib), (Ic), (Id), (Ie), (If), (Ig), (Ih), (Ii) and (Ij).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by androgen receptor, or (ii) associated with androgen receptor activity, or (iii) characterized by activity (normal or abnormal) of androgen receptor; or (2) modulating the activity of androgen receptor; or (3) modulating the expression of androgen receptor. In another non-limiting embodiment, the term “a therapeutically effective amount” refers to the amount of the compound of the present invention that, when administered to a cell, or a tissue, or a non-cellular biological material, or a medium, is effective to at least partially modulate the activity of androgen receptor; or at least partially modulate the expression of androgen receptor. The meaning of the term “a therapeutically effective amount” as illustrated in the above embodiment for the androgen receptor also applies by the same means to any other relevant proteins/peptides/enzymes, such as sex hormone-binding globulin (SHBG), or the putative testosterone-binding G-protein coupled receptor (GPRC6A), and the like.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Typically, a compound of formula (I) can be prepared according to the schemes provided infra.

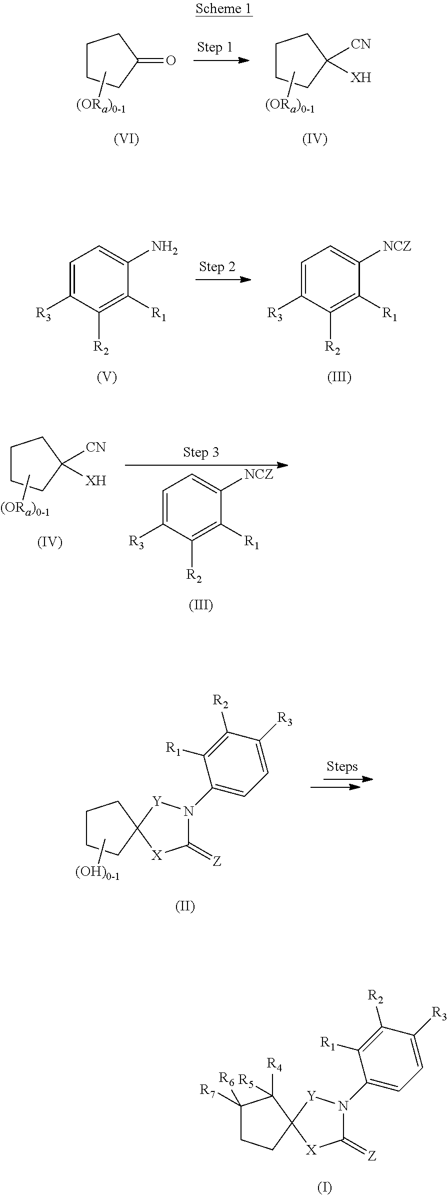

The process steps are described in more detail below:

### Step 1:

A compound of formula (IV) in which Rₐ represents a protecting group and X is as defined under formula (I) may be obtained by reaction of compound of formula (VI) in which Rₐ represents a protecting group, with a cyanating agent, e.g. trimethylsilylcyanide, optionally with a suitable amine e.g. methylamine, in a suitable solvent, e.g. tetrahydrofuran or DCM, optionally in the presence of a base, e.g. sodium sulphate. In the case where X is O, deprotection using suitable deprotecting agent may be used.

### Step 2:

A compound of formula (III) in which Z, R₁, R₂ and R₃ are as defined under formula (I) may be obtained by reaction of a compound of formula (V) with phosgene or thiophosgene in the presence of a suitable base, e.g. sodium hydrogen carbonate and in a suitable solvent, e.g. dichloromethane.

### Step 3:

A compound of formula (II) in which R₁, R₂, R₃, X, Y and Z are as defined under formula (I) may be obtained by treating a mixture of a compound of formula (IV) and a compound of formula (III) in a suitable solvent, e.g. dichloromethane, with a suitable base, e.g. triethylamine, to give, after reduction under pressure, a residue which is then heated in a suitable solvent, e.g. methanol, in the presence of a suitable acid, e.g. hydrochloric acid.

Compounds of formula (I) may be obtained from compounds of formula (II) prepared as described in Scheme 1 by further reduction, oxidation and/or other functionalisation of resulting compounds and/or by cleavage of any protecting group(s) optionally introduced.

Typically, a compound of formula (I′) can be prepared according to scheme 2 provided infra.

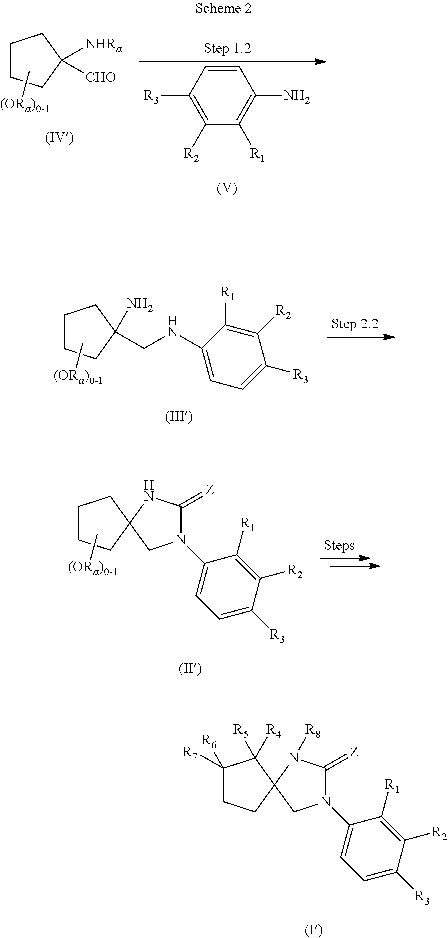

The process steps are described in more detail below:

### Step 1.2:

A compound of formula (III′) in which Rₐ represents a protecting group and R₁, R₂, R₃ are as defined under formula (I) may be obtained by reacting a compound of formula (IV′) in which Rₐ represents a protecting group with a compound of formula (V) in which R₁, R₂, R₃ are as defined under formula (I) in the presence of a reducing agent, e.g. sodium cyanoborohydride, in a suitable solvent, e.g. methanol and in the presence of a suitable acid, e.g. acetic acid, followed by deprotection using a suitable deprotecting agent, e.g. tetrabutylammoniumfluoride (TBAF) or trifluoroacetic acid (TFA), in a suitable solvent e.g. tetrahydrofuran (THF) or dichloromethane (DCM).

### Step 2.2:

A compound of formula (II′) in which in which Rₐ represents a protecting group and R₁, R₂, R₃ and Z are as defined under formula (I) may be obtained by reaction of a compound of formula (III′) with phosgene or thiophosgene in the presence of a suitable base, e.g. N,N diisopropylethylamine (DIPEA) in a suitable solvent, e.g. tetrahydrofuran (THF).

Compounds of formula (I′) in which R₁, R₂, R₃, R₄, R₅, R₆, R₇, R₈ and Z are as defined under formula (I) may be obtained from compounds of formula (II′) prepared as described in Scheme 2 by further reduction, oxidation and/or other functionalisation of resulting compounds and/or by cleavage of any protecting group(s) optionally introduced.

In a further aspect, the invention relates to a process for the preparation of a compound of formula (I), in free form or in pharmaceutically acceptable form, comprising the steps of:

a) coupling a compound of formula (IV) with a compound of formula (III) to form a spirocycle of formula (II);
b) the optional reduction, oxidation and/or other functionalization of the resulting compound of formula (II);
c) the cleavage of any protecting group(s) optionally present;
d) the recovery of the so obtainable compound of formula (I) in free form or in pharmaceutically acceptable salt form.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

In another aspect, the invention relates to a compound of formula (I′) in free form or in pharmaceutically acceptable salt form

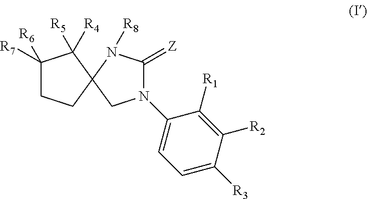

wherein Z, R₁, R₂, R₃, R₄, R₅, R₆, R₇ and R₈ are as defined herein with respect to a compound of formula (I).

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with or without a suitable permeation enhancer (including without limitation volatile or nonvolatile solvents) that improves the diffusion and solubility of the compound in the skin, other functional and non functional excipients (including without limiting, humectants, stabilizers, oils, surfactants, polymers, preservatives, antioxidants, moisturizers, emollients, solubilizers, penetration enhancers, skin protectants) and carriers suitable for transdermal delivery. The transdermal pharmaceutical compositions of the present invention can be made up in a semi-solid form (including without limitation gel, creams, ointments), solutions (including combination of several volatile and non volatile solvents and other pharmaceutical excipients) or solid (including without limitation reservoir patches, matrix patches, “patchless” formulations) comprising a backing member, a reservoir containing the compound optionally with carriers, optionally a rate controlling barrier to deliver the compound of the skin of the host at a controlled and predetermined rate over a prolonged period of time, and means to secure the device to the skin.

Moreover, administration through the skin by means of devices with or without the help of energy (including without limitation microneedle, iontophoresis, sonophoresis, thermal ablation) can be envisaged for delivery of the compound.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be desirable.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of the invention in free form or in salt form, exhibit valuable pharmacological properties, e.g. androgen receptor modulating properties, for example as indicated in in vitro tests as provided in the next sections and are therefore indicated for therapy or for use as research chemicals, e.g. tool compounds.

Compounds of the invention may be useful in the treatment or prevention of an indication selected from: muscular atrophy; lipodystrophy; long-term critical illness; sarcopenia; frailty or age-related functional decline; reduced muscle strength and function; reduced bone density or growth such as osteoporosis and osteopenia; the catabolic side effects of glucocorticoids; chronic fatigue syndrome; chronic myalgia; bone fracture; acute fatigue syndrome; muscle loss following elective surgery; cachexia; chronic catabolic state; eating disorders; side effects of chemotherapy; wasting secondary to fractures; wasting in connection with chronic obstructive pulmonary disease (COPD), chronic liver disease, AIDS, weightlessness, cancer cachexia, burn and trauma recovery, chronic catabolic state such as coma, eating disorders such as anorexia and chemotherapy; depression; nervousness; irritability; stress; growth retardation; reduced cognitive function; male contraception; hypogonadism; Syndrome X; diabetic complications or obesity.

In particular, compounds of the invention may be useful in the treatment or prevention of muscle wasting diseases, osteoporosis, sarcopenia, frailty, and cachexia such as AIDS cachexia, cancer cachexia, COPD cachexia.

Thus, as a further embodiment, the present invention provides the use of a compound of formula (I), (Ia), (Ib), (Ic), (Id), (Ie), (If), (Ig), (Ih), (Ii) and (Ij) in free from or in pharmaceutically acceptable salt form in therapy. In a further embodiment, the therapy is selected from a disease which may be treated by modulation of androgen receptor. In another embodiment, the disease is selected from the afore-mentioned list, suitably muscle wasting diseases, osteoporosis, sarcopenia, frailty, and cachexia, more suitably cancer cachexia and sarcopenia.

In another embodiment, the invention provides a method of treating a disease which is treated by modulation of androgen receptor comprising administration of a therapeutically acceptable amount of a compound of formula (I), (Ia), (Ib), (Ic), (Id), (Ie), (If), (Ig), (Ih), (Ii) and (Ij) in free from or in pharmaceutically acceptable salt form.

In a further embodiment, the disease is selected from the afore-mentioned list, suitably muscle wasting diseases, osteoporosis, sarcopenia, frailty, and cachexia, more suitably cancer cachexia and sarcopenia.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the present invention can be assessed by the following in vitro method. A method such as a modified Hershberger assay may be used to assess the activity of a compound of the invention in vivo.

### Test 1: In Vitro Assay

A suitable assay to determine the ability of a ligand to transcriptionally activate androgen receptor (AR) is carried out using mouse myoblastic C2C12 cells. The assay involves transfecting C2C12 cells with a plasmid containing full-length AR along with an AR response element linked to luciferase (2XIDR17). The luminescence read-out at the end of the assay is measured using Victor 3 and is a direct measure of the transcriptional activity. The assay has been validated using the reference compound, BMS-564929, for which EC₅₀ values have been reported in a similar set-up.

Preferred compounds of the invention have an EC50 value in the above-mentioned assay of less than 1 μM. More preferred compounds of the invention have an EC50 value in the above-mentioned assay of less than 100 nM. Even more preferred compounds of the invention have an EC50 value in the above-mentioned assay of less than 50 nM. Most preferred compounds of the invention have an EC50 value in the above-mentioned assay of less than 15 nM.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by androgen receptor modulation. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a pharmaceutical composition comprising a compound of formula (I) and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of formula (I).

In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula (I) for treating a disease or condition mediated by androgen receptor modulation, wherein the medicament is prepared for administration with another therapeutic agent. The invention also provides the use of another therapeutic agent for treating a disease or condition mediated by androgen receptor modulation, wherein the medicament is administered with a compound of formula (I).

The invention also provides a compound of formula (I) for use in a method of treating a disease or condition mediated by androgen receptor modulation, wherein the compound of formula (I) is prepared for administration with another therapeutic agent. The invention also provides another therapeutic agent for use in a method of treating a disease or condition mediated by androgen receptor modulation, wherein the other therapeutic agent is prepared for administration with a compound of formula (I). The invention also provides a compound of formula (I) for use in a method of treating a disease or condition mediated by androgen receptor modulation, wherein the compound of formula (I) is administered with another therapeutic agent. The invention also provides another therapeutic agent for use in a method of treating a disease or condition mediated by androgen receptor modulation, wherein the other therapeutic agent is administered with a compound of formula (I).

The invention also provides the use of a compound of formula (I) for treating a disease or condition mediated by androgen receptor modulation, wherein the patient has previously (e.g. within 24 hours) been treated with another therapeutic agent. The invention also provides the use of another therapeutic agent for treating a disease or condition mediated by androgen receptor modulation, wherein the patient has previously (e.g. within 24 hours) been treated with a compound of formula (I).

The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### EXAMPLES

### Abbreviations

Ac₂O acetic anhydride
AcOH acetic acid
AlBN azobisisobutyronitrile
Boc₂O di-tert-butyl dicarbonate
cm centimeters
COCl₂ phosgene
CuI copper iodide
d doublet
dd doublet of doublets
DAST diethylaminosulfurtrifluoride
DCM dichloromethane
DEA diethylamine
DIAD diisopropyl azodicarboxylate
DIBAL diisobutylaluminum hydride
DIPEA N,N-diisopropylethylamine
DMAP 4-Di(methylamino)pyridine
DMF N,N-dimethylformamide
DMSO dimethylsulfoxide
ee enantiomeric excess
ES electron-spray
EtOAc ethyl acetate
EtOH ethanol
g grams
h hour(s)
HCl hydrochloric acid
HPLC high pressure liquid chromatography
IPA isopropyl alcohol
IR infrared spectroscopy
LCMS liquid chromatography and mass spectrometry
1M one molar
MeI methyl iodide
MeOH methanol
MHz megahertz
MOM methoxymethyl
MS mass spectrometry
m multiplet
mbar millibar
min minutes
mL milliliter(s)
mmol millimole
MP melting point
m/z mass to charge ratio
N mol/L
NaH sodium hydride
NaHCO₃ sodium bicarbonate
Na₂SO₄ sodium sulfate
NBS N-bromosuccinimide
nm nanometer
nM nanomolar
NMR nuclear magnetic resonance
PCC pyridinium chlorochromate
PPh₃ triphenylphosphine
ppm parts per million
PPTS pyridinium p-toluenesulfonate
rt room temperature
RT retention time
s singlet
sat saturated
t triplet
TBAF tetrabutyl ammoniumfluoride
TBS t-butyl dimethylsilyl
TBDMS-Cl t-butyl dimethylsilyl chloride
TEA triethylamine
TFA trifluoroacetic acid
THF tetrahydrofuran
TLC thin layer chromatography
μm micrometers
wt weight

### Instruments Used:

NMR-400 MHz: Varian, Mercury
NMR-500 MHz: Varian, Unity INOVA
ES-MS: Applied Biosystems, API-3000
FT-IR: Shimadzu, IR Prestige 21

### Building block A1: 2-chloro-4-isothiocyanato-3-methylbenzonitrile

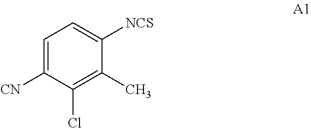

### 2-chloro-4-isothiocyanato-3-methylbenzonitrile (A1)

Thiophosgene (4.6 mL, 0.06 moles) was added dropwise to a stirred mixture of 4-amino-2-chloro-3-methylbenzonitrile (5.0 g, 0.03 moles) in dichloromethane (50 mL) and sodium hydrogen carbonate (5.04 g, 0.06 moles) at 0° C. The reaction mixture was allowed to warm to room temperature and stirred for 16 h. Once the starting material disappeared (monitored by TLC), the reaction mixture was filtered through celite. Filtrate was concentrated under reduced pressure to get the crude product which was purified by column chromatography (silica gel, 10% EtOAc in hexane) to provide the title compound.

Wt of the product: 4.5 g (72%)

¹H NMR (400 MHz, DMSO-d₆): δ 7.91 (d, J=8.4 Hz, 1H), 7.62 (d, J=8.2 Hz, 1H), 2.43 (s, 3H);

MS (ES): m/z 208.9 (M+1).

### Building block A2: 2-chloro-4-isocyanato-3-methylbenzonitrile

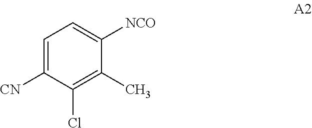

### 2-chloro-4-isocyanato-3-methylbenzonitrile (A2)

Phosgene (20%) in toluene (20.3 mL, 0.039 moles) was added drop wise to a stirred mixture of compound 4-amino-2-chloro-3-methylbenzonitrile (3.3 g, 0.02 moles) in dichloromethane (70 mL) and sodium hydrogen carbonate (3.3 g, 0.039 moles) at 0° C. Then reaction mixture was allowed to stir at room temperature and continued for 16 h. Once the starting material disappeared (monitored by TLC), reaction mixture was filtered by celite pad to remove sodium hydrogen carbonate. Filtrate was washed with saturated aqueous sodium hydrogen carbonate solution, dried over anhydrous sodium sulphate and concentrated under reduced pressure to get the crude product which was used in the next step without further purification.

Wt of the crude product: 3.0 g (78%)

¹H NMR (400 MHz, CDCl₃): δ 7.49 (d, J=8.3 Hz, 1H), 7.12 (d, J=8.3 Hz, 1H), 2.44 (s, 3H); MS (ES): m/z 190.9 (M−1).

### Building block B1: 2-(methoxymethoxy)-1-(methylamino)cyclopentanecarbonitrile

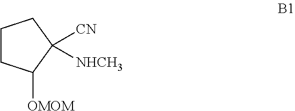

### a) cyclopentane-1,2-diol

To a stirred solution of cyclopentene (10 g, 0.147 moles) in acetone (100 mL) was added 50% aqueous 4-methyl morpholine-N-oxide (40 mL, 0.147 moles) followed by the addition of 2% osmium tetroxide in toluene at 0° C. and the reaction mixture was stirred for 16 h at room temperature. Once the starting material disappeared (monitored by TLC) the reaction mixture was quenched with saturated aqueous sodium meta-bisulphate and extracted with chloroform (3×300 mL). Chloroform layer was dried over anhydrous Na₂SO₄ and concentrated under reduced pressure to get the crude product which was used in the next step without further purification.

Wt of the crude product: 14 g (93%)

¹H NMR (400 MHz, CDCl₃): δ 4.06 (dd, J₁=3.9 Hz & J₂=8.3 Hz; 2H), 2.22-2.01 (m, 2H), 1.92-1.80 (m, 3H), 1.71-1.63 (m, 2H), 1.57-1.49 (m, 1H).

### b) 2-(methoxymethoxy)cyclopentanol

To a solution of cyclopentane-1,2-diol (14 g, 0.137 moles) in dichloromethane (140 mL) was added N-ethyldiisopropyl amine (36 mL, 0.206 moles) followed by the slow addition of chloromethylmethyl ether (10.42 mL, 0.137 moles) at 0° C. and the reaction mixture was stirred for 16 h at room temperature. Once the starting material disappeared (monitored by TLC), reaction mixture was diluted with dichloromethane, water and extracted. Organic layer was washed with water, dried over anhydrous Na₂SO₄ and concentrated under reduced pressure. Purification by column chromatography (silica gel, 30% EtOAc in hexane) provided the title compound.

Wt of the product: 8 g (40%)

¹H NMR (400 MHz, CDCl₃): δ 4.71 (d, J=2.0 Hz, 2H), 4.09-4.05 (m, 1H), 3.95-3.91 (m, 1H), 3.41 (s, 3H), 2.53 (d, J=4.3 Hz, 1H), 1.90-1.65 (m, 5H), 1.55-1.47 (m, 1H).

### c) 2-(methoxymethoxymethoxy)cyclopentanone

To a solution of 2-(methoxymethoxy)cyclopentanol (8 g, 0.055 moles) in acetone (80 mL) at 0° C. was added freshly prepared Jones' reagent (40 mL) drop wise. Then the reaction mixture was stirred at 0° C. for 6 h. Once the starting material disappeared (monitored by TLC), reaction mixture was diluted with ethyl acetate, water and extracted. Organic layer was washed with saturated aqueous sodium hydrogen carbonate, dried over Na₂SO₄ and concentrated under reduced pressure to get the crude product which was used in the next step without further purification.

Wt of the crude product: 5 g (63%)

¹H NMR (400 MHz, CDCl₃): δ 4.74 (d, J=6.9 Hz, 2H), 4.06-4.01 (m, 1H), 3.41 (s, 3H), 2.40-2.15 (m, 3H), 2.08-2.03 (m, 1H), 1.86-1.75 (m, 2H).

### d) 2-(methoxymethoxy)-1-(methylamino)cyclopentanecarbonitrile (B1)

Trimethylsilylcyanide (4.2 mL, 0.034 moles) was added drop wise to a stirred mixture of compound 2-(methoxymethoxy)cyclopentanone (4.0 g, 0.028 moles) in dry tetrahydrofuran (40 mL), 2M methylamine solution in tetrahydrofuran (14.0 mL, 0.028 moles) and sodium sulphate (19.9 g, 0.14 moles) at 0° C. Then the reaction mixture was allowed to warm to room temperature and stirred for 4 h. Once the starting material disappeared (monitored by TLC), the reaction mixture was filtered to remove sodium sulphate. Filtrate was diluted with ethyl acetate. Organic layer was washed with brine solution, dried over Na₂SO₄ and concentrated under reduced pressure to get the crude product which was used in the next step without further purification.

Wt of the crude product: 3.9 g (76%)

MS (ES): m/z 185.1 (M+1).

### Building block B2: tert-butyl(2-((tert-butyldimethylsilyl)oxy)-1-formylcyclopentyl)carbamate

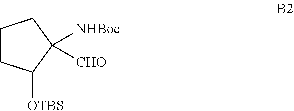 [SMILES: CC(C)(C)OC(=O)NC1(C=O)CCCC1O[Si](C)(C)C(C)(C)C]

### a) 2-(benzyloxy)cyclopentanol

To a suspension of NaH (0.392 g, 0.009 moles) in dry THF (10 mL) was added diol (as obtained in the procedure described for building block B1, step a) in THF (1 g, 9 mmol) at 0° C. and stirred for 10 minutes. Then the solution of benzyl bromide in THF (1.0 ml, 8 mmol) was added followed by tetra butyl ammonium iodide at 0° C. and stirred at ambient temperature for 24 h. Once the starting material disappeared (monitored by TLC), reaction mixture was quenched with ammonium chloride and extracted with ethyl acetate. Organic layer was washed with water, dried over anhydrous Na₂SO₄ and concentrated under reduced pressure. Purification by column chromatography (silica gel, 7% EtOAc in hexane) provided the title compound.

Wt of the product: 1.2 g (63%)

¹H NMR (400 MHz, DMSO): δ 7.37-7.23 (m, 5H), 4.59 (d, J=11.8 Hz, 1H), 4.47 (d, J=12.2 Hz, 1H), 4.24 (d, J=4.4 Hz, 1H), 4.00-3.95 (m, 1H), 3.66-3.62 (m, 1H), 1.75-1.40 (m, 6H).

### b) 2-(benzyloxy)cyclopentanone

To a solution of 2-(benzyloxy)cyclopentanol (3.9 g, 0.020 moles) in acetone (60 mL) at 0° C. was added freshly prepared Jones' reagent (12 mL) drop wise. The reaction mixture was stirred at 0° C. for 2 h. Once the starting material disappeared (monitored by TLC), reaction mixture was diluted with ethyl acetate, water and extracted. Organic layer was washed with saturated aqueous sodium hydrogen carbonate, dried over Na₂SO₄ and concentrated under reduced pressure. Purification by column chromatography (silica gel, 10% EtOAc in hexane) provided the title compound.

Wt of the product: 1.9 g (49%)

¹H NMR (400 MHz, DMSO): δ 7.37-7.28 (m, 5H), 4.69 (d, J=12.2 Hz, 1H), 4.57 (d, J=11.8 Hz, 1H), 3.94-3.89 (m, 1H), 2.26-2.14 (m, 3H), 1.98-1.66 (m, 3H).

### c) 6-(benzyloxy)-1,3-diazaspiro[4.4]nonane-2,4-dione

To a stirred solution of (NH₄)₂CO₃ (51.71 g, 0.342 moles) and NH₄Cl (7.31 g, 0.136 moles) in water (250 mL), 2-(benzyloxy)cyclopentanone (6.5 g, 0.034 moles) in ethanol (250 ml) was added and stirred at room temperature for 15 min. Then NaCN (8.38 g, 0.171 moles) was added and the reaction mixture was stirred at 100° C. for 48 h. Once the starting material disappeared (monitored by TLC), the reaction mixture was quenched with saturated ferrous sulphate solution and extracted with ethyl acetate (3×50 mL). Ethyl acetate layer was washed with brine solution and dried over anhydrous Na₂SO₄ and concentrated under reduced pressure. Purification by column chromatography (silica gel, 5% MeOH in DCM) provided the title compound.

Wt of the product: 5.4 g (60%)

¹H NMR (400 MHz, DMSO): δ 10.63 (s, 1H), 8.24 (s, 1H), 7.35-7.26 (m, 5H), 4.49-4.38 (m, 2H), 3.96-3.92 (m, 1H), 2.05-1.94 (m, 2H), 1.79-1.56 (m, 4H).

MS (ES): m/z 259 [M−1].

### d) 2-(benzyloxy)-1-((tert-butoxycarbonyl)amino)cyclopentanecarboxylic acid

To 6-(benzyloxy)-1,3-diazaspiro[4.4]nonane-2,4-dione (5.4 g, 0.020 moles) in sealed tube, 3N NaOH solution (180 mL) was added and stirred at 100° C. for 19 h. Once the starting material disappeared (monitored by TLC), reaction mixture pH was adjusted to 6-7 with concentrated HCl and solvent was removed under reduced pressure. The orange color residue was extracted with hot methanol twice and methanol was evaporated under reduced pressure. The residue was dissolved in methanol (220 mL), and Et₃N (45 mL) was added followed by (Boc)₂O (10.06 mL, 0.045 moles) and reaction mixture was stirred at room temperature for 18 h. Once the starting material disappeared (monitored by TLC), the solvent was removed under reduced pressure. Purification by column chromatography (silica gel, 4% MeOH in DCM) provided the title compound.

Wt of the product: 5.2 g (75%)

¹H NMR (400 MHz, CDCl₃): δ 7.37-7.29 (m, 5H), 5.75 (s, 1H), 4.58 (s, 2H), 4.43 (s, 1H), 2.35-2.26 (m, 2H), 2.10-1.92 (m, 2H), 1.82-1.62 (m, 2H), 1.46 (s, 9H).

### e)methyl 2-(benzyloxy)-1-((tert-butoxycarbonyl)amino)cyclopentanecarboxylate

To a stirred solution of 2-(benzyloxy)-1-((tert-butoxycarbonyl)amino)cyclopentanecarboxylic acid (5.2 g, 15.5 mmol) in ether (100 mL) was added (100 mL) of diazomethane (prepared from 8 g of nitrosoethylurea) at 0° C. and the reaction mixture was stirred for 30 minutes. Once the starting material disappeared (monitored by TLC), the solvent was removed under reduced pressure to get the crude product. Purification by column chromatography (silica gel, 1% MeOH in DCM) provided the title compound.

Wt of the product: 3.6 g (66%)

¹H NMR (400 MHz, CDCl₃): δ 7.36-7.28 (m, 5H), 5.52 (s, 1H), 4.57-4.46 (m, 2H), 4.06-4.05 (m, 1H), 3.72 (s, 3H), 2.38 (m, 2H), 2.05-2.01 (m, 2H), 1.88-1.70 (m, 2H), 1.56 (s, 9H).

### f)methyl 1-((tert-butoxycarbonyl)amino)-2-hydroxycyclopentanecarboxylate

To a stirred solution of methyl 2-(benzyloxy)-1-((tert-butoxycarbonyl)amino)cyclopentanecarboxylate (3.6 g, 10 mmol) in MeOH (45 mL) was added 10% Pd/C (3.6 g) and stirred for 3 h at room temperature under hydrogen atmosphere. Once the starting material disappeared (monitored by TLC), the residue was filtered off from the reaction mixture through a celite bed and the filtrate was concentrated under reduced pressure to get the crude product which was used in the next step without further purification.

Wt of the product: 2.19 g (82%)

¹H NMR (400 MHz, CDCl₃): δ 5.38 (s, 1H), 4.3 (s, 1H), 3.74 (s, 3H), 3.49 (m, 1H), 2.40-2.21 (m, 1H), 2.17-2.10 (m, 2H), 1.79-1.60 (m, 3H), 1.58 (s, 9H).

### g)methyl 1-((tert-butoxycarbonyl)amino)-2-((tert-butyldimethylsilyl)oxy)cyclopentanecarboxylate

To a stirred solution of methyl 1-((tert-butoxycarbonyl)amino)-2-hydroxycyclopentanecarboxylate (2.19 g, 8 mmol) in dry DMF (40 mL) was added imidazole (1.72 g, 25 mmol) at 0° C. and the reaction mixture was stirred for 15 minutes followed by TBS-Cl addition at 0° C., then the reaction mixture was slowly allowed to warm to room temperature and stirred for 24 h. Once the starting material disappeared (monitored by TLC), water was added and extracted with ethyl acetate (3×50 mL). The organic layer was washed with water, brine solution, dried over Na₂SO₄ and concentrated under reduced pressure. Purification of the residue by column chromatography (silica gel, 10% EtOAc in hexane) provided the title compound.

Wt of the product: 2.70 g (85%)

¹H NMR (400 MHz, CDCl₃): δ 5.43 (s, 1H), 4.22 (s, 1H), 3.69 (s, 3H), 2.39 (s, 2H), 1.98-1.95 (m, 1H), 1.81-1.60 (m, 4H), 1.44 (s, 9H), 0.88 (s, 9H), 0.014 (s, 6H).

### h) tert-butyl(2-((tert-butyldimethylsilyl)oxy)-1-formylcyclopentyl)carbamate (B2)

To a stirred solution of methyl 1-((tert-butoxycarbonyl)amino)-2-((tert-butyldimethylsilyl)oxy)cyclopentanecarboxylate (2.7 g, 7.2 mmol) in DCM (60 mL) was added DIBAL in toluene (14.47 mL, 14.4 mmol) at −78° C. and the reaction mixture was stirred for 2 h. Once the starting material disappeared (monitored by TLC), the reaction mixture was quenched with sodium potassium tartarate solution and extracted with ethyl acetate (3×100 mL). Ethyl acetate layer was washed with brine solution and dried over anhydrous Na₂SO₄ and concentrated under reduced pressure. Purification by column chromatography (silica gel, 7% ethyl acetate in hexane) provided the title compound.

Wt of the product: 1.4 g (56%)

¹H NMR (400 MHz, CDCl₃): δ 9.60 (s, 1H), 5.56 (s, 1H), 4.15 (m, 1H), 2.24-1.60 (m, 6H), 1.45 (s, 9H), 0.89 (s, 9H), 0.05 (s, 6H).

### Building block B3: 1-(methylamino)cyclopentanecarbonitrile

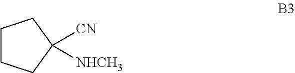

The title compound was synthesized using analogous procedure to building block B1, step d, starting from cyclopentanone.

Wt of the crude product: 1.5 g (100%).

### Building block B4: 1-((2-methoxyethyl)amino)cyclopentanecarbonitrile

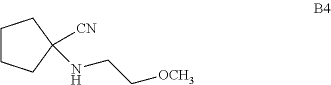 [SMILES: COCCNC1(C#N)CCCC1]

The title compound was synthesized using analogous procedure to building block B1, step d, starting from cyclopentanone.

Wt of the crude product: 0.9 g (90%).

### Building block B5: 1-(ethylamino)cyclopentanecarbonitrile

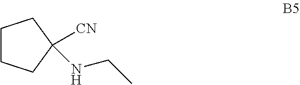

The title compound was synthesized using analogous procedure to building block B1, step d, starting from cyclopentanone.

Wt of the crude product: 0.8 g (97%).

### Building block B6: 3-(methoxymethoxy)-1-(methylamino)cyclopentanecarbonitrile

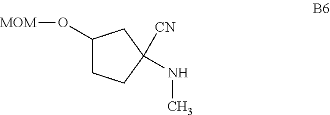

### a) 3-(methoxymethoxy)cyclopentanol

The title compound was synthesized using analogous procedure to building block B1, step b.

Wt of the product: 2.0 g (56%)

### b) 3-(methoxymethoxy)cyclopentanone

The title compound was synthesized using analogous procedure to building block B1, step c.

Wt of the crude product: 1.5 g (76%).

### c) 3-(methoxymethoxy)-1-(methylamino)cyclopentanecarbonitrile (B6)

The title compound was synthesized using analogous procedure to building block B1, step d.

Wt of the crude product: 1.0 g (52%)

### Building block B7: 1-hydroxy-2-(methoxymethoxy)cyclopentanecarbonitrile

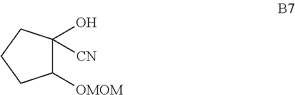

### a) 2-(methoxymethoxy)-1-((trimethylsilyl)oxy)cyclopentanecarbonitrile

Trimethylsilyl cyanide (1.3 mL, 10.4 mmol) was added drop wise to a stirred mixture of 2-(methoxymethoxy)cyclopentanol (1.0 g, 6.94 mmol) (obtained as described in building block B1, step c) in dry dichloromethane (20 mL), N-methylmorpholine N-oxide (0.244 g, 2.08 mmol) at room temperature and continued for 12 h. Once the starting material disappeared (monitored by TLC), reaction mixture was diluted with dichloromethane, water and extracted. Organic layer was washed with brine solution, dried over Na₂SO₄ and concentrated under reduced pressure to get the crude product which was purified by column chromatography (silica gel, 5% EtOAc in hexane) to provide the title compound.

Weight of the product: 0.83 g (50%)

¹H NMR (400 MHz, CDCl₃): δ 4.81-4.67 (m, 2H), 4.12-4.02 (m, 1H), 3.42-3.36 (m, 3H), 2.17-2.0 (m, 3H), 1.80-1.66 (m, 3H), 0.25 (s, 9H).

### b) 1-hydroxy-2-(methoxymethoxy)cyclopentanecarbonitrile

To a stirred mixture of 2-(methoxymethoxy)-1-((trimethylsilyl)oxy)cyclopentanecarbonitrile (0.83 g, 3.41 mmol) in ethylacetate (10 mL), 2N HCl (3.5 mL) was added dropwise at 0° C. and stirring was continued for 3.5 h at room temperature. Once the starting material disappeared (monitored by TLC), reaction mixture was diluted with ethylacetate, water and extracted. Organic layer was washed with brine solution, dried over Na₂SO₄ and concentrated under reduced pressure to get the crude product which was directly used for the next step.

Weight of the product: 0.33 g (57%)

¹H NMR (400 MHz, CDCl₃): δ 4.75-4.70 (m, 2H), 4.24-4.20 (t, J=7.8 Hz, 1H), 3.44 (s, 3H), 1.98-1.80 (m, 3H), 1.79-1.66 (m, 3H).

### Building block B8: 4-(((1-cyanocyclopentyl)amino)methyl)benzonitrile

### a) 4-(azidomethyl)benzonitrile

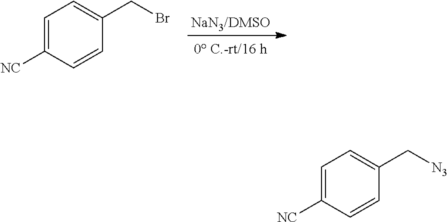 [SMILES: N#Cc1ccc(CBr)cc1 ; N#Cc1ccc(CN=[N+]=[N-])cc1]

Sodium azide (2.5 g, 0.04 moles) was added portionwise to a stirred mixture of 4-cyano benzylbromide (5.0 g, 0.03 moles) in DMSO (50 mL) at 0° C. The reaction mixture was stirred at room temperature for 16 h. Once the starting material was consumed (monitored by TLC), the reaction mixture was quenched with cold water and extracted with ethyl acetate (3×150 mL). Organic layer was washed with water, brine solution, dried over Na₂SO₄ and concentrated under reduced pressure. Crude compound was obtained as pale yellow liquid (3.88 g) which was used in the next step without further purification.

### b) 4-(aminomethyl)benzonitrile

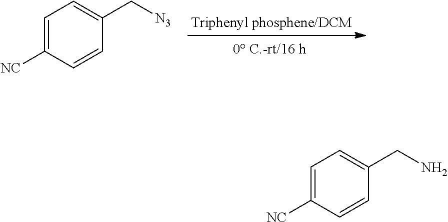 [SMILES: N#Cc1ccc(CN=[N+]=[N-])cc1 ; N#Cc1ccc(CN)cc1]

Triphenyl phosphine (2.25 g, 0.009 moles) was added portion wise to a stirred mixture of 4-(azidomethyl)benzonitrile as obtained in step a) (0.88 g, 0.006 moles) in dichloromethane (10 mL) at 0° C. The reaction mixture was stirred at room temperature for 16 h. Once the starting material was consumed (monitored by TLC), the reaction mixture was quenched with cold water and the residue was extracted with dichloromethane (3×50 mL). The organic layer was washed with water, brine solution, dried over Na₂SO₄ and concentrated under reduced pressure. Purification by column chromatography (silica gel, 4% MeOH in chloroform) provided the title compound as pale yellow colored gummy compound (0.43 g, 56%).

¹H NMR (400 MHz, CDCl₃): δ 7.63 (d, J=8.3 Hz, 2H), 7.45 (d, J=8.2 Hz, 2H), 3.96 (s, 2H);

### c) 4-(((1-cyanocyclopentyl)amino)methyl)benzonitrile

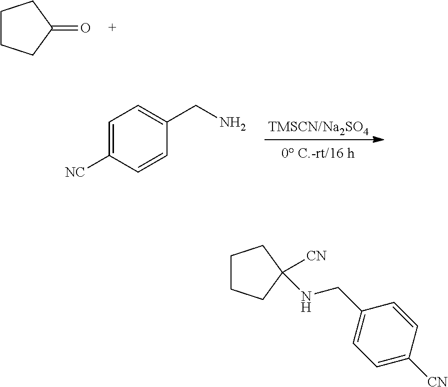

The title compound was synthesized using same procedure used for 1-(methylamino)cyclopentanecarbonitrile (B3) using 4-(aminomethyl)benzonitrile and cyclopentanone as starting materials. The crude product (0.77 g) was obtained as brown liquid which was not further purified.

### Building block B9: 1-(((5-methylisoxazol-3-yl)methyl)amino)cyclopentanecarbonitrile

### a) 3-(azidomethyl)-5-methylisoxazole

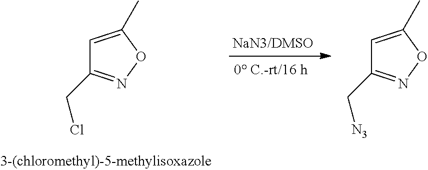

The title compound was synthesized using same procedure used for 4-(azidomethyl)benzonitrile (building block B8 step a) using 3-(chloromethyl)-5-methylisoxazole as starting material. The crude compound was obtained as pale yellowish liquid (0.21 g) which was used in the next step without further purification.

### b) (5-methylisoxazol-3-yl)methanamine

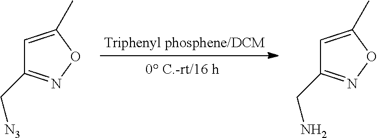

The title compound was synthesized using same procedure used for building block B8, step b) using 3-(azidomethyl)-5-methylisoxazole as starting material. Purification by column chromatography (silica gel, 4% MeOH in dichloromethane) provided the title compound as light brown semi solid (0.125 g, 73%).

MS (LC-MS): m/z 113.2 (M+1);

### c) 1-(((5-methylisoxazol-3-yl)methyl)amino)cyclopentanecarbonitrile (R9)

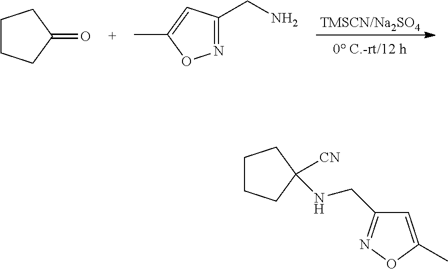

The title compound was synthesized using same procedure used for building block B3 using (5-methylisoxazol-3-yl)methanamine and cyclopentanone as starting material. The crude product was obtained as brown liquid (0.22 g) which was not further purified.

MS (LC-MS): m/z 206.2 (M+1).

### Building block B10: 1-((2-(pyridin-4-yl)ethyl)amino)cyclopentanecarbonitrile

### a) 2-(pyridin-4-yl)ethanol

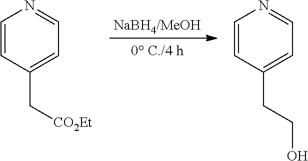 [SMILES: CCOC(=O)Cc1ccncc1 ; OCCc1ccncc1]

Sodium borohydride (0.7 g, 0.02 moles) was added portion wise to a stirred mixture of (1.0 g, 0.006 moles) in methanol (10 mL) at 0° C. The reaction mixture was stirred for 4 h at 0° C. Once the starting material was consumed (monitored by TLC), reaction mixture was quenched with saturated ammonium chloride solution and extracted with dichloromethane (3×50 mL). Organic layer was washed with brine solution, dried over Na₂SO₄ and concentrated under reduced pressure to get the crude product (0.65 g) as brown liquid which was used in the next step without further purification.

¹H NMR (400 MHz, DMSO): δ 8.45-8.43 (dd, J, =8.0 Hz & J₂=2.4 Hz; 2H), 7.25-7.24 (dd, J, =4.4 Hz & J₂=1.5 Hz; 2H), 4.72-4.69 (t, J=5.1 Hz; 1H), 3.67-3.62 (dt, J, =5.4 Hz & J₂=1.5 Hz; 2H), 2.74-2.71 (t, J=6.6 Hz; 2H);

MS (ES-MS): m/z 124.0 (M+1);

### b) 4-(2-bromoethyl)pyridine

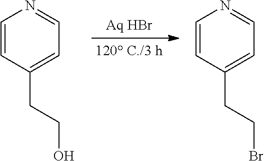 [SMILES: OCCc1ccncc1 ; BrCCc1ccncc1]

Aqueous hydrobromic acid (3.5 mL) was added drop wise to 2-(pyridin-4-yl)ethanol at room temperature and it was heated slowly to 120° C. The reaction mixture was stirred for 3 h at 120° C. Once the starting material consumed (monitored by TLC), reaction mixture was poured into crushed ice and extracted with ethyl acetate (3×15 mL). Organic layer was washed with aqueous sodium bicarbonate solution, brine solution, dried over Na₂SO₄ and concentrated under reduced pressure to get the crude product as pale yellow liquid (0.5 g) which was used in the next step without further purification.

### c) 4-(2-azidoethyl)pyridine

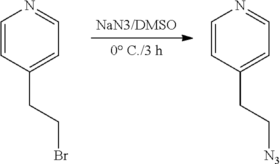 [SMILES: BrCCc1ccncc1 ; [N-]=[N+]=NCCc1ccncc1]

The title compound was synthesized using similar procedure which was used for the synthesis of building block B8, step a) using 4-(2-bromoethyl)pyridine as the starting material. The crude compound was obtained as brown liquid (0.15 g) and used in the next step without further purification.

MS (LC-MS): m/z 149.1 (M+1);

### d) 2-(pyridin-4-yl)ethanamine

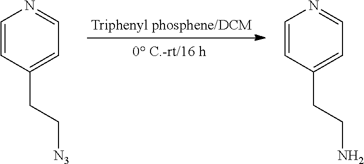 [SMILES: [N-]=[N+]=NCCc1ccncc1 ; NCCc1ccncc1]

The title compound was synthesized using similar procedure which was used for the synthesis of building block B8 step b) using 4-(2-azidoethyl)pyridine as the starting material. The crude product was obtained as cream color semi solid (0.07 g) which was used in the next step without further purification.

MS (LC-MS): m/z 123.2 (M+1).

### e) 1-((2-(pyridin-4-yl)ethyl)amino)cyclopentanecarbonitrile

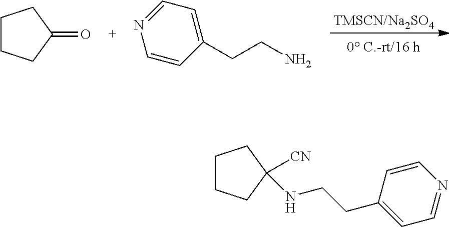

The title compound was synthesized using similar procedure which was used for the synthesis of building block B3 using 2-(pyridin-4-yl)ethanamine and cyclopentanone as the starting material. The crude product was obtained as brown liquid (0.11 g) which was not further purified.

MS (LC-MS): m/z 216.2 (M+1).

### Building block B11: 1-((2-fluoroethyl)amino)cyclopentanecarbonitrile

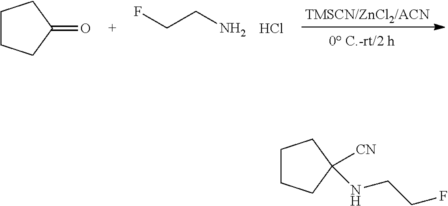

Zinc chloride (0.035 mg, 0.0003 moles) was added to a stirred mixture of cyclopentanone (0.11 mL, 1 mmol) in acetonitrile, 2-fluoroethanamine hydrochloride (0.25 mg, 3 mmol) and trimethyl silylcyanide (0.31 mL, 3 mmol) at 0° C. The reaction mixture was stirred at room temperature for 2 h. Once the starting material was consumed (monitored by TLC), the reaction mixture was quenched with aqueous ammonia and the residue was extracted with ethyl acetate (3×25 mL). Organic layer was washed with water, brine solution, dried over Na₂SO₄ and concentrated under reduced pressure to get the crude product as pale brown liquid (0.11 g) which was used in the next step without further purification.

MS (LC-MS): m/z 157.2 (M+1).

### Building block B12: 1-((5-cyanopentyl)amino)cyclopentanecarbonitrile

### a) 6-azidohexanenitrile

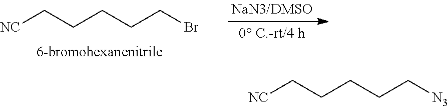 [SMILES: N#CCCCCCBr ; N#CCCCCCN=[N+]=[N-]]

The title compound was synthesized using similar procedure used for the synthesis of building block B8 step a) using 6-bromohexanenitrile as the starting material. The crude compound was obtained as colorless liquid (0.7 g) and used in the next step without further purification.

### b) 6-aminohexanenitrile

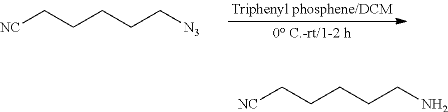 [SMILES: N#CCCCCCN=[N+]=[N-] ; N#CCCCCCN]

The title compound was synthesized using similar procedure used for the synthesis of building block B8 step b) using 6-azidohexanenitrile as starting material. Crude product was obtained as cream color semi solid (0.5 g) which was used in the next step without further purification.

### c) 1-((5-cyanopentyl)amino)cyclopentanecarbonitrile (B12)

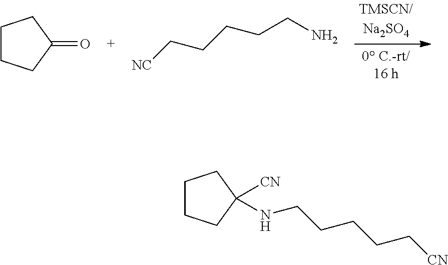 [SMILES: O=C1CCCC1 ; N#CCCCCCN ; N#CCCCCCNC1(C#N)CCCC1]

The title compound was synthesized using similar procedure used for the synthesis of building block B3 using 6-aminohexanenitrile as the starting material. The crude product was obtained as brown liquid which was used in the next step without further purification (0.98 g).

### Building block B13: 1-((2-(2-fluoroethoxyl)ethyl)amino)cyclopentanecarbonitrile

### a) tert-butyl(2-hydroxyethyl)carbamate

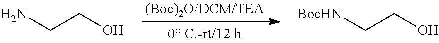

BOC anhydride (28.0 mL, 0.12 moles) was added to stirred solution of 2-aminoethanol (5.0 g, 0.08 moles) in dichloromethane (50 mL) and triethyl amine (22.7 mL, 0.16 moles) at 0° C. The reaction mixture was stirred at room temperature for 12 h. Once the starting material was consumed (monitored by TLC), the reaction mixture was diluted with water and the residue was extracted with ethyl acetate (3×150 mL). The organic layer was washed with water, brine solution, dried over Na₂SO₄ and concentrated under reduced pressure. Purification by column chromatography (silica gel, 20% EtOAc in hexane) provided the title compound as colorless liquid (4.0 g, 30%).

¹H NMR (400 MHz, DMSO): δ 6.66 (s, 1H), 4.56-4.54 (t, J=6.0 Hz, 1H), 3.37-3.32 (m, 2H), 2.99-2.94 (q, J=5.9 Hz; 2H), 1.37 (s, 9H);

### b) tert-butyl(2-(2-fluoroethoxy)ethyl)carbamate

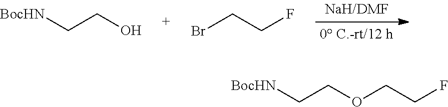

To a stirred solution of sodium hydride (0.5 g, 0.01 moles) in DMF (10 mL) was added tert-butyl(2-hydroxyethyl)carbamate (1.0 g, 0.006 moles) followed by 1-bromo-2-fluoroethane (0.95 g, 0.007 moles) at 000° C. The reaction mixture was stirred for 12 h at room temperature. Once the starting material was consumed (monitored by TLC), the reaction mixture was diluted with cold water and extracted with ethyl acetate (2×50 mL).

The organic layer was washed with water, brine solution, dried over Na₂SO₄ and concentrated under reduced pressure. Purification by column chromatography (silica gel, 10% EtOAc in hexane) provided the title compound as colorless liquid (0.5 g, 39%).

¹H NMR (400 MHz, DMSO): δ 6.78 (s, 1H), 4.57-4.43 (m, 2H), 3.66-3.56 (m, 2H), 3.42-3.39 (t, J=5.8 Hz; 2H), 3.31-3.05 (m, 2H), 1.37 (s, 9H);

### c) 2-(2-fluoroethoxy)ethanamine

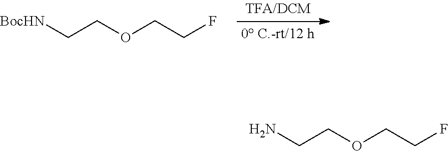 [SMILES: CC(C)(C)OC(=O)NCCOCCF ; NCCOCCF]

Trifluoroaceticacid (1.0 mL) was added to a stirred solution of tert-butyl(2-(2-fluoroethoxy)ethyl)carbamate (0.8 g, 0.004 moles) in DCM (10 mL) at 0° C. The reaction mixture was stirred for 12 h at room temperature. Once the starting material was consumed (monitored by TLC), the reaction mixture was concentrated under reduced pressure. The crude product was obtained as brown liquid (0.5 g) which was used in the next step without further purification.

### d) 1-((2-(2-fluoroethoxyl)ethyl)amino)cyclopentanecarbonitrile B13

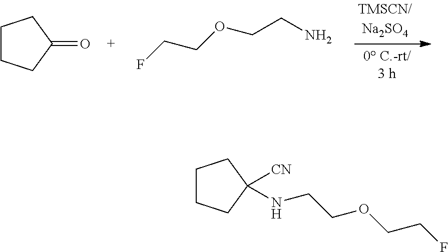 [SMILES: O=C1CCCC1 ; NCCOCCF ; N#CC1(NCCOCCF)CCCC1]

The title compound was synthesized using similar procedure used for the synthesis of building block B3 using 2-(2-fluoroethoxyl)ethanamine and cyclopentanone as the starting materials. The crude product was obtained as brown liquid (0.25 g) which was not further purified.

### Building block B14: 1-((2-(cyanomethoxy)ethyl)amino)cyclopentanecarbonitrile

### a) tert-butyl(2-(cyanomethoxy)ethyl)carbamate

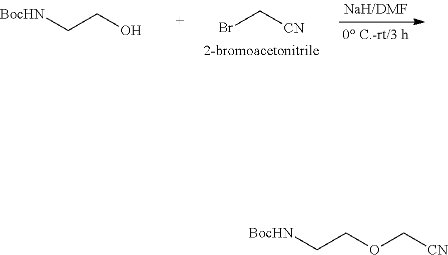

The title compound was synthesized using similar procedure used for building block B13 step b) using 2-bromoacetonitrile and tert-butyl(2-hydroxyethyl)carbamate as starting material. Purification by column chromatography (silica gel, 8% EtOAc in hexane) provided the title compound as colorless liquid (0.5 g, 40%).

¹H NMR (400 MHz, DMSO): δ 6.88 (s, 1H), 4.45 (s, 2H), 3.50-3.47 (t, J=5.9 Hz; 2H), 3.12-3.08 (m, 2H), 1.37 (s, 9H);

### b) 2-(2-aminoethoxy)acetonitrile

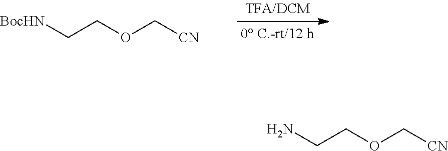 [SMILES: CC(C)(C)OC(=O)NCCOCC#N ; N#CCOCCN]

The title compound was synthesized using similar procedure used for the synthesis of building block B13 step c) using tert-butyl(2-(cyanomethoxy)ethyl)carbamate as starting material. The crude product was obtained as colorless liquid (0.51 g) which was used in the next step without further purification.

### c) 1-((2-(cyanomethoxy)ethyl)amino)cyclopentanecarbonitrile (B14)

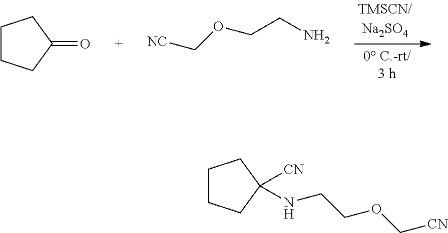 [SMILES: O=C1CCCC1 ; N#CCOCCN ; N#CCOCCNC1(C#N)CCCC1]

The title compound was synthesized using similar procedure used for building block B3 using 2-(2-aminoethoxy)acetonitrile and cyclopentanone as the starting material. The crude product was obtained as brown liquid (0.5 g) which was used in the next step without further purification.

### Building block B15: 6-((methoxymethoxy)methyl)-1,3-diazaspiro[4.4]nonane-2,4-dione

### a)ethyl 1,4-dioxaspiro[4.4]nonane-6-carboxylate

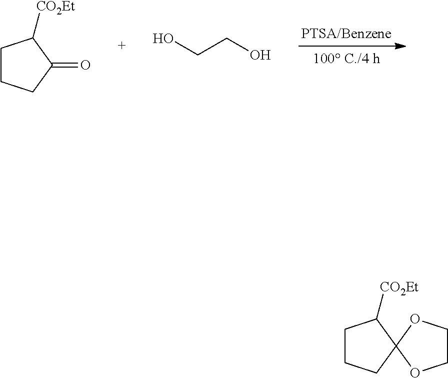

P-toluene sulphonic acid (1.1 g, 0.006 moles) was added to a stirred solution of ethyl-2-oxocyclopentanecarboxylate (10.0 g, 0.06 moles) in benzene (50 mL) followed by ethane-1,2-diol (50 g, 0.8 moles) at room temperature. The reaction mixture was heated to reflux with dean stark apparatus and stirred for 4 h. Once the starting material was consumed (monitored by TLC), the reaction mixture was diluted with cold water. The organic layer was washed with saturated aqueous sodium bicarbonate solution, water, brine solution, dried over Na₂SO₄ and concentrated under reduced pressure. Purification by column chromatography (silica gel, 10% EtOAc in hexane) provided the title compound as colorless liquid (7.1 g, 55%).

¹H NMR (400 MHz, DMSO): δ 4.10-4.01 (m, 2H), 3.99-3.77 (m, 4H), 2.85-2.81 (t, J=8.8 Hz; 1H), 1.97-1.82 (m, 2H), 1.78-1.68 (m, 3H), 1.57-1.52 (m, 1H), 1.22-1.16 (t, J=6.9 Hz; 3H);

MS (LC-MS): m/z 201.2 (M+1).

### b) 1,4-dioxaspiro[4.4]nonan-6-ylmethanol

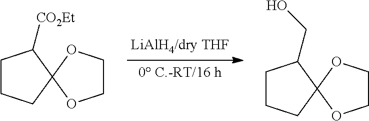 [SMILES: CCOC(=O)C1CCCC12OCCO2 ; OCC1CCCC12OCCO2]

To a stirred solution of lithium aluminium hydride (1.3 g, 0.03 moles) in dry tetrahydro furan (50 mL) was added ethyl 1,4-dioxaspiro[4.4]nonane-6-carboxylate as obtained in step a) (7.0 g, 0.03 moles) at 0° C. The reaction mixture was stirred for 16 h at room temperature. Once the starting material was consumed (monitored by TLC), the reaction mixture was quenched by the drop wise addition of aqueous NaOH solution at 0° C. and the formed salts were filtered. The filtrate was diluted with ethyl acetate (50 mL) and washed with water, brine solution, dried over Na₂SO₄ and concentrated under reduced pressure. Purification by column chromatography (silica gel, 25% EtOAc in hexane) provided the title compound as colorless liquid (4.6 g, 77%).

¹H NMR (400 MHz, DMSO): δ 4.26-4.23 (t, J=5.4 Hz; 1H), 3.84-3.76 (m, 4H), 3.51-3.46 (m, 1H), 3.25-3.20 (m, 1H), 1.99-1.94 (m, 1H), 1.83-1.79 (m, 1H), 1.67-1.41 (m, 5H);

MS (LC-MS): m/z 201.2 (M+1).

### c) 6-((methoxymethoxy)methyl)-1,4-dioxaspiro[4.4]nonane

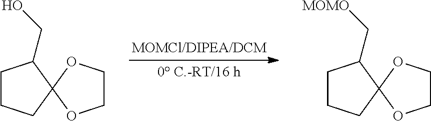

To a solution of 1,4-dioxaspiro[4.4]nonan-6-ylmethanol as obtained in step b) (3.5 g, 0.02 moles) in dichloromethane (50 mL) was added N-ethyldiisopropyl amine (5.5 mL, 0.03 moles) followed by the drop wise addition of chloromethylmethyl ether (1.9 mL, 0.02 moles) at 0° C. and the reaction mixture was stirred for 16 h at room temperature. Once the starting material was consumed (monitored by TLC), reaction mixture was diluted with dichloromethane, water and extracted. Organic layer was washed with water, dried over anhydrous Na₂SO₄ and concentrated under reduced pressure. Purification by column chromatography (silica gel, 10% EtOAc in hexane) provided the title compound as colorless liquid (2.2 g, 50%).

¹H NMR (400 MHz, DMSO): δ 4.55-4.51 (q, J=6.3 Hz; 2H), 3.85-3.78 (m, 4H), 3.53-3.48 (m, 1H), 3.33 (s, 3H), 3.31-3.27 (m, 1H), 2.13-2.10 (m, 1H), 1.88-1.84 (m, 1H), 1.70-1.40 (m, 5H);

### d) 2-((methoxymethoxy)methyl)cyclopentanone

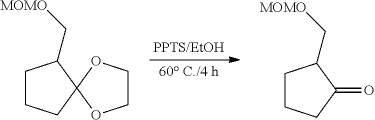

Pyridinium p-toluene sulfonate (0.51 g, 0.002 moles) was added to a stirred solution of 6-((methoxymethoxy)methyl)-1,4-dioxaspiro[4.4]nonane as obtained in step c) (2.2 g, 0.01 moles) in ethanol (30 mL) at room temperature. The reaction mixture was heated to 60° C. and the reaction mixture was stirred for 4 h. Once the starting material was consumed (monitored by TLC), the reaction mixture was diluted with water and extracted with ethyl acetate (100 mL). The organic layer was washed with water, dried over anhydrous Na₂SO₄ and concentrated under reduced pressure. Purification by column chromatography (silica gel, 15% EtOAc in hexane) provided the title compound as colorless liquid (1.1 g, 64%).

¹H NMR (400 MHz, DMSO): δ 4.53-4.51 (m, 2H), 3.83-3.79 (m, 1H), 3.61-3.53 (m, 2H), 3.23 (s, 3H), 2.37-2.33 (m, 1H), 2.20-2.02 (m, 3H), 1.94-1.91 (m, 1H), 1.83-1.81 (m, 2H);

### e) 6-((methoxymethoxy)methyl)-1,3-diazaspiro[4.4]nonane-2,4-dione (B15)

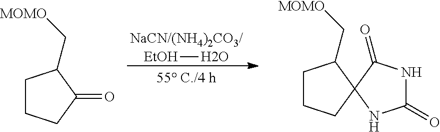

To a stirred solution of ammonium carbonate (8.5 g, 0.09 moles) in water (25 mL), 2-((methoxymethoxy)methyl)cyclopentanone as obtained in step d) (2.0 g, 0.01 moles) in ethanol (25 ml) was added and the reaction mixture stirred at room temperature for 15 min. Sodium cyanide (1.24 g, 0.02 moles) was added and the reaction mixture was stirred at 55° C. for 4 h. Once the starting material was consumed (monitored by TLC), the reaction mixture was diluted with water and extracted with ethyl acetate (3×150 mL). Ethyl acetate layer was washed with brine solution and dried over anhydrous Na₂SO₄ and concentrated under reduced pressure. Crude product was obtained as pale yellow liquid (1.4 g) which was used in the next step without further purification.

MS (LC-MS): m/z 159.1 (M+1).

### Building block B16: 1-(((3,5-dimethylisoxazol-4-yl)methyl)amino)cyclopentanecarbonitrile

### a) 4-(azidomethyl)-3,5-dimethylisoxazole

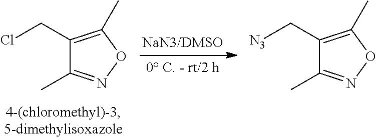

The title compound was synthesized using similar procedure used for synthesizing R3 using 4-(chloromethyl)-3,5-dimethylisoxazole as the starting material. The crude compound was obtained as pale yellowish liquid (0.15 g) which was used in the next step without further purification.

¹H NMR (400 MHz, DMSO): δ 4.30 (s, 2H), 2.40 (s, 3H), 2.20 (s, 3H);

MS (LC-MS): m/z 153.1 (M+1).

### b) (3,5-dimethylisoxazol-4-yl)methanamine

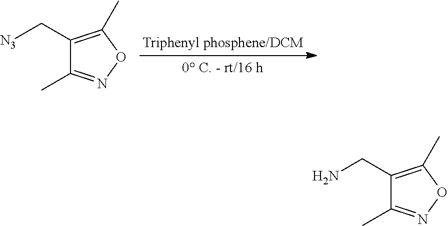

The title compound was synthesized using similar procedure used for building block B8 step b) using 4-(azidomethyl)-3,5-dimethylisoxazole as obtained in step a) as the starting material. Purification by column chromatography (silica gel, 20% EtOAc in hexane) provided the title compound as an off-white solid (0.1 g, 80%).

¹H NMR (400 MHz, DMSO): δ 3.40 (s, 2H), 2.31 (s, 3H), 2.19 (s, 3H);

MS (ES-MS): m/z 126.9 (M+1).

### c) 1-(((3,5-dimethylisoxazol-4-yl)methyl)amino)cyclopentanecarbonitrile (B16)

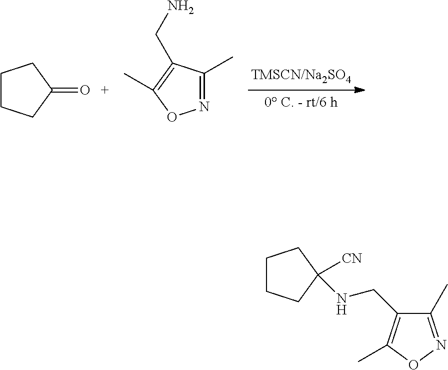

The title compound was synthesized using similar procedure used for building block B3 using (3,5-dimethylisoxazol-4-yl)methanamine as obtained in step b) as the starting material. The crude product was obtained as brown liquid (0.15 g) which was not further purified.

### Building block B17: 1-((pyridin-2-ylmethyl)amino)cyclopentanecarbonitrile

### a) 2-(azidomethyl)pyridine

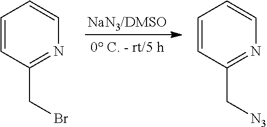 [SMILES: BrCc1ccccn1 ; [N-]=[N+]=NCc1ccccn1]

The title compound was synthesized using an analogous procedure to building block B8 step a). The crude compound was obtained as gummy solid (0.23 g) and used in the next step without further purification.

¹H NMR (400 MHz, CDCl₃): δ 8.61 (d, J=4.4 Hz; 1H), 7.75-7.70 (m, 1H), 7.35 (d, J=7.8 Hz; 1H), 7.27-7.24 (m, 1H), 4.49 (s, 2H).

### b) pyridin-2-ylmethanamine

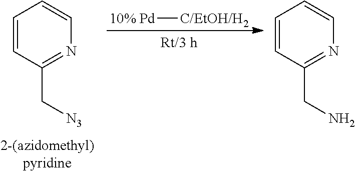

10% Palladium charcoal (0.05 g) was added to a stirred solution of 2-(azidomethyl)pyridine as obtained in step a) (0.22 g, 0.002 moles) at room temperature. The reaction mixture was stirred under hydrogen atmosphere for 3 h. Once the starting material was consumed (monitored by TLC), the reaction mixture was filtered through celite. The filtrate was concentrated under reduced pressure. The crude product was obtained as gummy solid (0.12 g) which was used in the next step without further purification.

¹H NMR (400 MHz, DMSO): δ 8.54 (t, J=6.6 Hz; 1H), 7.82-7.76 (m, 1H), 7.52-7.47 (m, 1H), 7.31-7.24 (m, 1H), 4.11-4.06 (m, 1H), 3.88 (br s, 1H), 3.84 (m, 1H);

MS (ES-MS): m/z 109.1 (M+1).

### c) 1-((pyridin-2-ylmethyl)amino)cyclopentanecarbonitrile (B17)

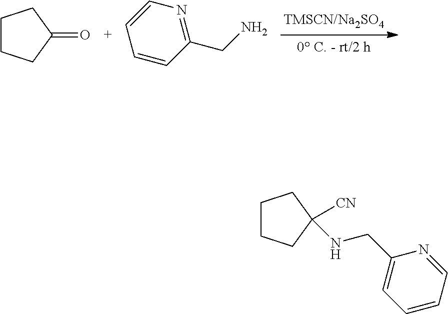

The title compound was synthesized using analogous procedure used for the synthesis of building block B3 using pyridin-2-ylmethanamine and cyclopentanone as the starting materials. The crude product was obtained as gummy liquid (0.11 g) which was used without further purification.

MS (ES-MS): m/z 202.1 (M+1).

### Building block B18: 1-((pyridin-4-ylmethyl)amino)cyclopentanecarbonitrile

### a) 4-(azidomethyl)pyridine

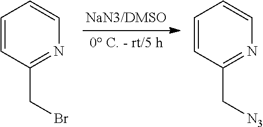 [SMILES: BrCc1ccccn1 ; [N-]=[N+]=NCc1ccccn1]

The title compound was synthesized using analogous procedure to building block B8 step a). The crude compound was obtained as gummy solid (0.3 g) and used in the next step without further purification.

MS (ES-MS): m/z 135.1 (M+1).

### b) pyridin-4-ylmethanamine

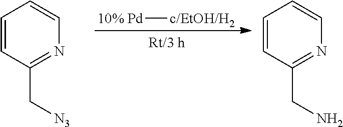

The title compound was synthesized using analogous procedure used for the synthesis of building block B17 step b) using 4-(azidomethyl)pyridine as obtained in step a) as the starting material. The crude product was obtained as gummy liquid (0.23 g) which was used in the next step without further purification.

MS (ES-MS): m/z 108.9 (M+1).

### c) 1-((pyridin-4-ylmethyl)amino)cyclopentanecarbonitrile (B18)

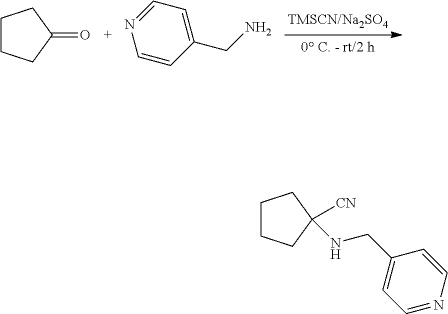

The title compound was synthesized using analogous procedure used for the synthesis of building block B3 using pyridin-4-ylmethanamine as obtained in step b) and cyclopentanone as the starting material. Crude product was obtained as gummy liquid (0.15 g) which was used without further purification.

MS (ES-MS): m/z 202.1 (M+1).

### Building block B19: 1-((3-cyanopropyl)amino)cyclopentanecarbonitrile

### a) 4-azidobutanenitrile

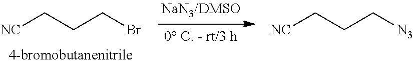 [SMILES: N#CCCCBr ; N#CCCCN=[N+]=[N-]]

The title compound was synthesized using analogous procedure used for building block B8 step a) using 4-bromobutanenitrile as the starting material. The crude compound was obtained as a colorless liquid (0.4 g) and used in the next step without further purification.

¹H NMR (400 MHz, CDCl₃): δ 3.50 (t, J=6.3 Hz, 2H), 2.61-2.46 (m, 2H), 1.95-1.88 (m, 2H).

### b) 4-aminobutanenitrile

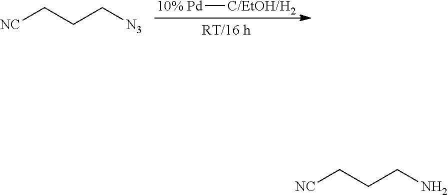

The title compound was synthesized using analogous procedure used for R45 using 4-azidobutanenitrile as the starting material. The crude product was obtained as a colorless liquid (0.14 g) which was used in the next step without further purification.

MS (LC-MS): m/z 85.1 (M+1).

### c) 1-((3-cyanopropyl)amino)cyclopentanecarbonitrile (B19)

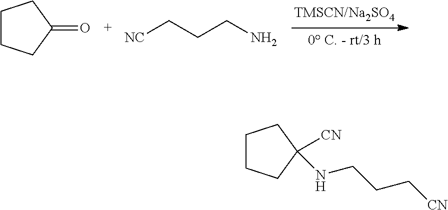 [SMILES: O=C1CCCC1 ; N#CCCCN ; N#CCCCNC1(C#N)CCCC1]

The title compound was synthesized using analogous procedure used for building block B3 using 4-aminobutanenitrile as obtained in step b) and cyclopentanone as the starting material. The crude product was obtained as a colorless liquid (0.1 g) which was used without further purification.

### Building block B20: 1-(isobutylamino)cyclopentanecarbonitrile (B20)

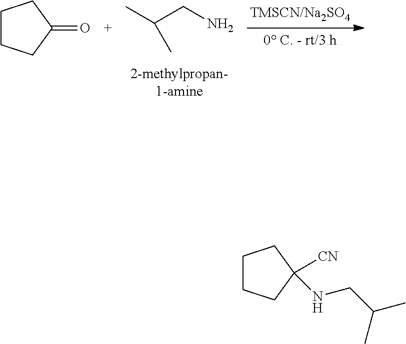

The title compound was synthesized using analogous procedure used for building block B3 using 2-methylpropan-1-amine and cyclopentanone as the starting materials. The crude product was obtained as a colorless liquid (0.45 g) which was used without further purification.

### Building block B21: 1-(((6-methylpyridin-3-yl)methyl)amino)cyclopentanecarbonitrile

### a)methyl 6-methylnicotinate

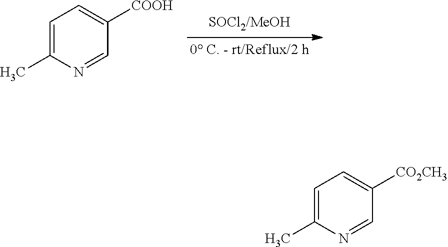

To a stirred solution of 6-methylnicotinic acid (2.1 g, 0.01 moles) in methanol (25 mL) was added dropwise thionylchloride (2.3 mL, 0.03 moles) at 0° C. The reaction mixture was allowed to come to room temperature and then heated to reflux. The reaction mixture was stirred for 2 h at reflux. Once the starting material was consumed (monitored by TLC), the reaction mixture was concentrated under reduced pressure. The residue was diluted with water and extracted with ethyl acetate (2×100 mL). The organic layer was washed with water, dried over anhydrous Na₂SO₄ and concentrated under reduced pressure. The crude product was obtained as gummy liquid (1.84 g) which was used in the next step without further purification.

¹H NMR (400 MHz, DMSO): δ 8.96 (s, 1H), 8.18-8.16 (dd, J, =1.8 Hz & J₂=7.8 Hz; 1H), 7.42 (d, J=8.3 Hz; 1H), 3.87 (s, 3H), 2.55 (s, 3H);

MS (ES-MS): m/z 152.1 (M+1).

### b) (6-methylpyridin-3-yl)methanol

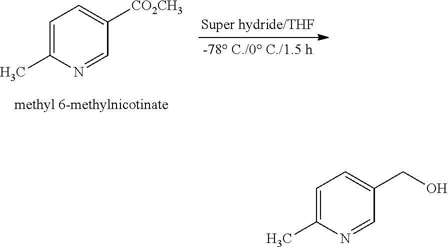

1M Lithium triethylborohydride (super hydride) in THF (12.0 mL, 0.01 moles) was added to a stirred solution of methyl 6-methylnicotinate (0.9 g, 0.06 moles) as obtained in step a) in dry THF (10 mL) at −78° C. The reaction mixture was slowly allowed to rise to 0° C. and stirred for 1.5 h. Once the starting material was consumed (monitored by TLC), the reaction mixture was quenched with saturated aqueous ammonium chloride solution and extracted with ethyl acetate (2×100 mL). The organic layer was washed with water, dried over anhydrous Na₂SO₄ and concentrated under reduced pressure. Purification by column chromatography (silica gel, 3% MeOH in dichloromethane) provided the title compound as pale yellow solid (0.61 g, 84%).

¹H NMR (400 MHz, DMSO): δ 8.37 (s, 1H), 7.57 (dd, J, =2.0 Hz & J₂=7.9 Hz; 1H), 7.19 (d, J=7.8 Hz; 1H), 5.21 (t, J=5.9 Hz; 1H), 4.47 (d, J=5.4 Hz; 2H), 2.43 (s, 3H);

MS (ES-MS): m/z 124.0 (M+1).

### c) 5-(bromomethyl)-2-methylpyridine

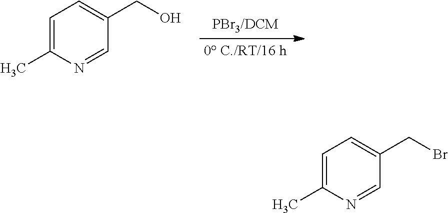

To a stirred solution of (6-methylpyridin-3-yl)methanol (0.28 g, 3 mmol) as obtained in step b) in DCM (10 mL) at 0° C. was added PBr₃ (0.48 mL, 5 mmol) and the reaction mixture was stirred at 25° C. for 16 h. Once the starting material was consumed (monitored by TLC), the reaction mixture was extracted with DCM. The organic layer was washed with sat. sodium bicarbonate solution, water, brine, dried over Na₂SO₄ and concentrated to get the desired product (420 mg) as brown liquid which was used in the next step without further purification.

### d) 5-(azidomethyl)-2-methylpyridine

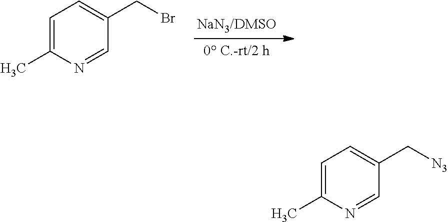

The title compound was synthesized using analogous procedure used for building block B8 step a) using 5-(bromomethyl)-2-methylpyridine as obtained in step c) as the starting material. Purification by column chromatography (silica gel, 25% EtOAc in hexane) provided the title compound as a gummy liquid (0.23 g, 68%).

¹H NMR (400 MHz, CDCl₃): δ 8.45 (s, 1H), 7.51-7.50 (m, 1H), 7.19 (d, J=8.3 Hz, 1H), 4.34 (s, 2H), 2.57 (s, 3H);

MS (ES-MS): m/z 149.3 (M+1).

### e) (6-methylpyridin-3-yl)methanamine

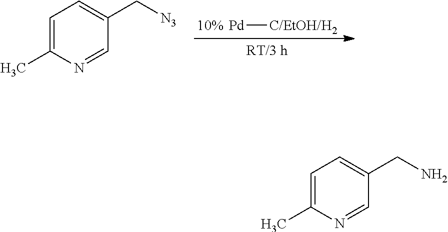

The title compound was synthesized using analogous procedure used for building block B17 step b) using 5-(azidomethyl)-2-methylpyridine as obtained in step d) as the starting material. The crude product was obtained as a gummy liquid (0.09 g) which was used in the next step without further purification.

¹H NMR (400 MHz, CDCl₃): δ 8.44 (s, 1H), 7.58-7.54 (m, 1H), 7.12 (d, J=7.9 Hz, 1H), 3.86-3.77 (m, 2H), 2.54 (s, 3H);

MS (LC-MS): m/z 123.0 (M+1).

### f) 1-(((6-methylpyridin-3-yl)methyl)amino)cyclopentanecarbonitrile (B21)

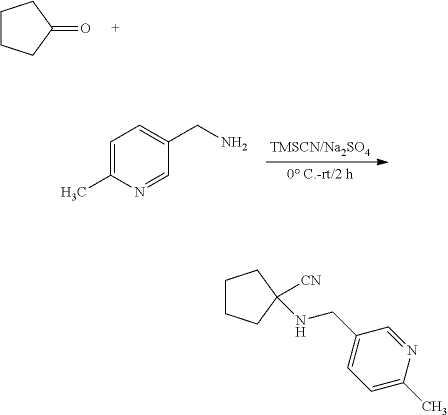

The title compound was synthesized using analogous procedure used for building block B3 using (6-methylpyridin-3-yl)methanamine as obtained in step e) and cyclopentanone as the starting materials. The crude product was obtained as a gummy liquid (0.16 g) which was used without further purification.

### Building block B22: 1-((4-cyanobutyl)amino)cyclopentanecarbonitrile

### a) 5-azidopentanenitrile

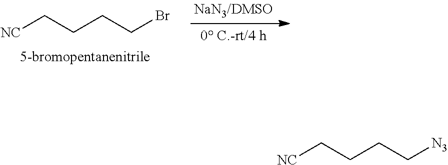 [SMILES: N#CCCCCBr ; N#CCCCCN=[N+]=[N-]]

The title compound was synthesized using analogous procedure used for building block B8 step a) using 5-bromopentanenitrile as the starting material. The crude compound was obtained as a gummy liquid (2.0 g) and used in the next step without further purification.

### b) 5-aminopentanenitrile

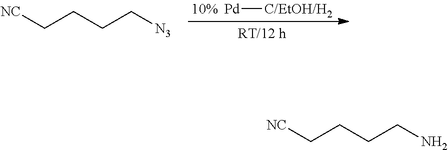

The title compound was synthesized using analogous procedure used for building block B17 step b) using 5-azidopentanenitrile as obtained in step a) as the starting material. The crude product was obtained as a gummy solid (1.6 g) which was used in the next step without further purification.

MS (ES-MS): m/z 98.9 (M+1).

### c) 1-((4-cyanobutyl)amino)cyclopentanecarbonitrile (B22)

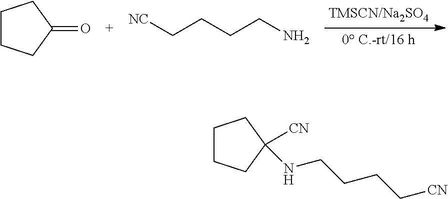 [SMILES: O=C1CCCC1 ; N#CCCCCN ; N#CCCCCNC1(C#N)CCCC1]

The title compound was synthesized using analogous procedure used for building block B3 using 5-aminopentanenitrile as obtained in step b) and cyclopentanone as the starting materials. The crude product was obtained as a gummy liquid (2.95 g) which was used without further purification.

### Building block B23: 1-((2-((tert-butyldimethylsilyl)oxy)ethyl)amino)cyclopentanecarbonitrile

### a) 2-((tert-butyldimethylsilyl)oxy)ethanamine

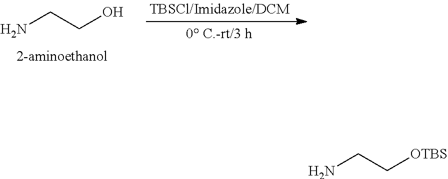

To a stirred solution of 2-aminoethanol (10 g, 0.16 moles) in DCM (90 mL) at 0° C. was added TBDMS-Cl (37 g, 0.25 moles), followed by the addition of imidazole (16.7 g, 0.25 moles). The reaction mixture was stirred at rt for 3 h. Once the starting material was consumed (monitored by TLC), the reaction mixture was extracted with DCM. The organic phase was washed with water, brine then dried over Na₂SO₄ and concentrated. Purification by column chromatography (silica gel, 2% methanol in DCM) provided the title compound (14.8 g, 52%) as a colorless gummy solid.

¹H NMR (400 MHz, DMSO): δ 3.50 (t, J=5.9 Hz; 2H), 2.58-2.50 (m, 2H), 0.86 (s, 9H), 0.03 (s, 6H);

MS (ES-MS): m/z 176.3 (M+1).

### b) 1-((2-((tert-butyldimethylsilyl)oxy)ethyl)amino)cyclopentanecarbonitrile (B23)

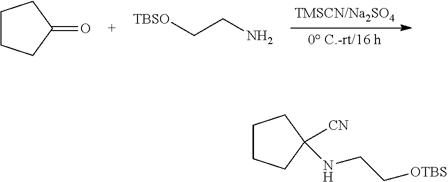 [SMILES: O=C1CCCC1 ; CC(C)(C)[Si](C)(C)OCCN ; CC(C)(C)[Si](C)(C)OCCNC1(C#N)CCCC1]

The title compound was synthesized using analogous procedure used for building block B3 using 2-((tert-butyldimethylsilyl)oxy)ethanamine as obtained in step a) and cyclopentanone as the starting material. The crude product was obtained as a gummy liquid (1.8 g) which was used in the next step without further purification.

### Building block B24: 1-((2-cyanoethyl)amino)cyclopentanecarbonitrile

### a) 3-azidopropanenitrile

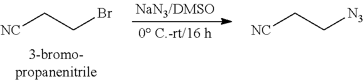 [SMILES: N#CCCBr ; N#CCCN=[N+]=[N-]]

The title compound was synthesized using an analogous procedure used for building block B8 step a) using 3-bromopropanenitrile as the starting material. The crude compound was obtained as a gummy liquid (0.75 g) and used in the next step without further purification.

### b) 3-aminopropanenitrile

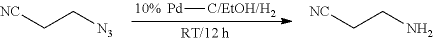

The title compound was synthesized using an analogous procedure used for building block B17 step b) using 3-azidopropanenitrile as obtained in step a) as the starting material. The crude product was obtained as a gummy solid (0.18 g) which was used in the next step without further purification.

MS (ES-MS): m/z 69.0 (M−1).

### c) 1-((2-cyanoethyl)amino)cyclopentanecarbonitrile (B24)

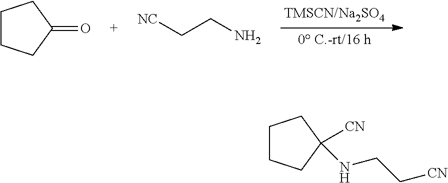 [SMILES: O=C1CCCC1 ; N#CCCN ; N#CCCNC1(C#N)CCCC1]

The title compound was synthesized using analogous procedure used for building block B3 using 3-aminopropanenitrile as obtained in step b) and cyclopentanone as the starting materials. The crude product was obtained as a gummy liquid (0.3 g) which was used in the next step without further purification.

### Building block B25: 1-((3-fluoropropyl)amino)cyclopentanecarbonitrile

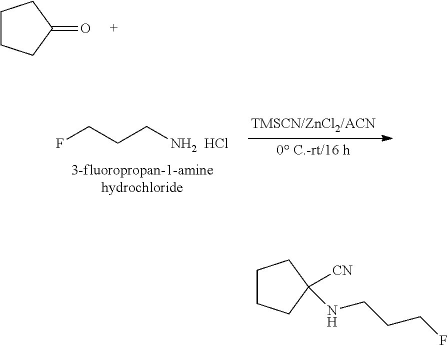 [SMILES: O=C1CCCC1 ; NCCCF ; Cl ; N#CC1(NCCCF)CCCC1]

The title compound was synthesized using analogous procedure used for building block B11 using 3-fluoropropan-1-amine hydrochloride and cyclopentanone as the starting materials. The crude product was obtained as a gummy liquid (0.11 g) which was used without further purification.

MS (LC-MS): m/z 171.2 (M+1).

### Building block B26: 2-methyl-1-(methylamino)cyclopentanecarbonitrile

### a)ethyl 1-methyl-2-oxocyclopentanecarboxylate

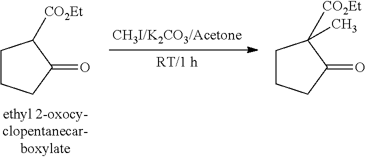

To a stirred solution of ethyl 2-oxocyclopentanecarboxylate (1.0 g, 6 mmol) in acetone (5 mL) was added potassium carbonate (2.65 g, 20 mmol) followed by methyl iodide (0.83 mL, 10 mmol) at room temperature. The reaction mixture was stirred for 1 h at rt. Once the starting material was consumed (monitored by TLC), the reaction mixture was diluted with water and extracted with ethyl acetate (2×100 mL). The organic layer was washed with water, dried over anhydrous Na₂SO₄ and concentrated under reduced pressure. Purification by column chromatography (silica gel, 10% EtOAc in hexane) provided the title compound as a pale yellow liquid (0.2 g, 20%).

¹H NMR (400 MHz, CDCl₃): δ 4.19-4.11 (m, 2H), 2.54-2.40 (m, 2H), 2.35-2.27 (m, 1H), 2.09-2.02 (m, 1H), 1.97-1.82 (m, 2H), 1.24 (s, 3H), 1.22 (s, 3H);

MS (ES-MS): m/z 171.1 (M+1).

### b) 2-methylcyclopentanone

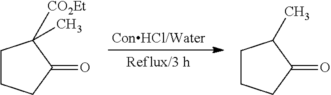

Concentrated hydrochloric acid (20 mL) was added to a stirred solution of ethyl 1-methyl-2-oxocyclopentanecarboxylate (9.1 g, 0.05 moles) as obtained in step a) in water (10 mL) at rt. The reaction mixture was heated to reflux and stirred at same temperature for 3 h. Once the starting material was consumed (monitored by TLC), the reaction mixture was diluted with water and extracted with diethyl ether (2×200 mL). The organic layer was washed with water, dried over anhydrous Na₂SO₄ and concentrated under reduced pressure. The crude product was obtained as a light yellow liquid (4.7 g) which was used in the next step without further purification.

¹H NMR (400 MHz, CDCl₃): δ 2.34-2.20 (m, 2H), 2.17-2.10 (m, 2H), 2.09-1.95 (m, 1H), 1.85-1.71 (m, 1H), 1.53-1.43 (m, 1H), 1.09 (d, J=6.8 Hz; 3H);

### c) 2-methyl-1-(methylamino)cyclopentanecarbonitrile (B26)

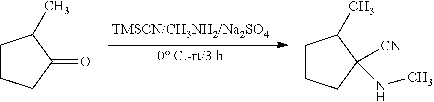

The title compound was synthesized using analogous procedure used for building block B3 using 2-methylcyclopentanone as obtained in step b) as the starting material. The crude product was obtained as light yellow liquid (2.8 g) which was used without further purification.

### Building block B27: 1-(((5-methyloxazol-2-yl)methyl)amino)cyclopentanecarbonitrile

### a)ethyl 2-((2-hydroxypropyl)amino)-2-oxoacetate

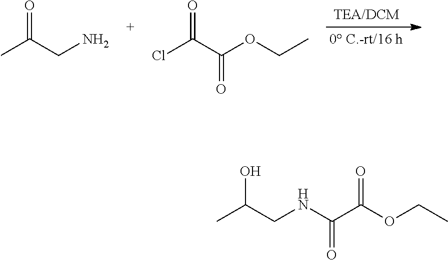

To a stirred solution of 1-aminopropan-2-ol (3.0 gm, 39.9 mmol) in DCM (30 mL) was added triethylamine (8.4 ml, 59.9 mmol) at 0° C. then ethyl 2-chloro-2-oxoacetate (4.46 ml, 39.9 mmol) was added at the same temperature and stirring was continued at rt for 16 h. Once the starting material was consumed (monitored by TLC), the reaction mixture was extracted with DCM and washed with water and brine. The organic layer was dried over sodium sulphate, concentrated. Purification by column chromatography (silica gel, 70% EtOAc in hexane) provided the title compound (800 mg, 12%).

MS (ES): m/z: 174 (M−1).

### b)ethyl 2-oxo-2-((2-oxopropyl)amino)acetate

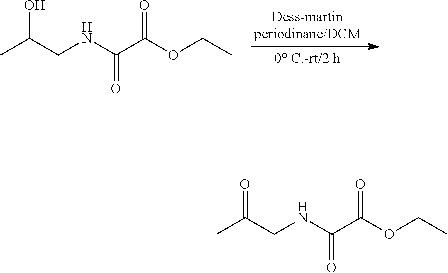

To a stirred solution of ethyl 2-((2-hydroxypropyl)amino)-2-oxoacetate (0.8 g, 4.5 mmol) as obtained in step a) in DCM (15 mL) was added Dess-martin periodinane (1.93 g, 4.5 mmol) at 0° C. and the reaction mixture was stirred at rt for 2 h. Once the starting material was consumed (monitored by TLC), the reaction mixture was extracted with ethylacetate and washed with sodiumbicarbonate solution and brine. The organic phase was dried over sodium sulphate, concentrated. Purification by column chromatography (silica gel, 60% EtOAc in hexane) provided the title compound (500 mg, 63%).

MS (ES): m/z: 172 (M−1).

### c)ethyl 5-methyloxazole-2-carboxylate

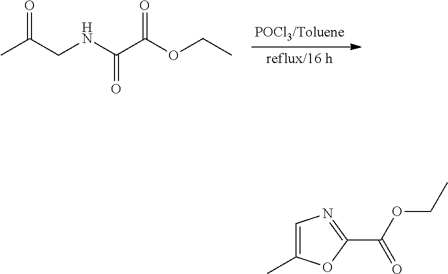

To a stirred solution of ethyl 2-oxo-2-((2-oxopropyl)amino)acetate (0.5 g, 2.8 mmol) as obtained in step b) in toluene (5 mL) was added phosphorusoxychloride (0.26 mL, 2.5 mmol) at rt. The reaction mixture was heated to reflux for 16 hr. Once the starting material was consumed (monitored by TLC), the reaction mixture was cooled to rt and extracted with ethyl acetate, washed with water, saturated sodium bicarbonate solution and brine. The organic phase was dried over sodium sulphate, concentrated. Purification by column chromatography (silica gel, 15% EtOAc in hexane) provided the title compound (300 mg, 60%).

¹H NMR (400 MHz, DMSO-d₆): 7.17 (s, 1H), 4.32 (q, J=6.9 Hz, 2H), 2.39 (s, 3H), 1.31 (t, J=7.3 Hz, 3H); MS (ES): m/z: 156 (M+1).

### d) (5-methyloxazol-2-yl)methanol

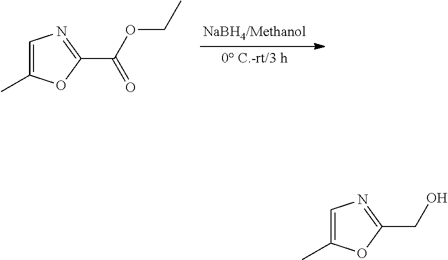

To a stirred solution of ethyl 5-methyloxazole-2-carboxylate (300 mg, 1.9 mmol) as obtained in step c) in methanol (10 mL) was added sodiumborohydride (183 mg, 4.8 mmol) at 0° C. The reaction mixture was stirred at rt for 3 h. Once the starting material was consumed (monitored by TLC), the reaction mixture was extracted with chloroform and washed with water and brine. The organic phase was dried over sodium sulphate, concentrated to give the crude product (180 mg) which was used in the next step without further purification.

¹H NMR (400 MHz, DMSO-d₆): δ 6.75 (s, 1H), 5.54 (t, J=5.8 Hz, 1H), 4.41 (d, J=5.9 Hz, 2H), 2.27 (s, 3H); MS (ES): m/z: 114 (M+1).

### e) 2-(bromomethyl)-5-methyloxazole

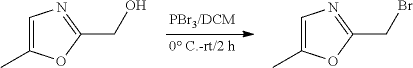

To a stirred solution of (5-methyloxazol-2-yl)methanol (500 mg, 4.4 mmol) as obtained in step d) in DCM (15 mL) at 0° C. was added PBr₃ (0.68 mL, 6.6 mmol) and the reaction mixture was stirred at 25° C. for 2 h. Once the starting material was consumed (monitored by TLC), the reaction mixture was extracted with DCM. The organic layer was washed with saturated sodium bicarbonate solution, water, brine, dried over Na₂SO₄ and concentrated to get the desired product (620 mg) as a brown liquid which was used in the next step without further purification.

### f) 2-(azidomethyl)-5-methyloxazole

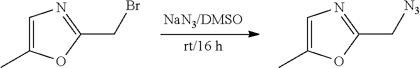

The title compound was synthesized using an analogous procedure used for building block B8 step a) using 2-(bromomethyl)-5-methyloxazole as obtained in step e) as the starting material. The crude compound was obtained as a colorless liquid (300 mg) and used in the next step without further purification.

### g) (5-methyloxazol-2-yl)methanamine

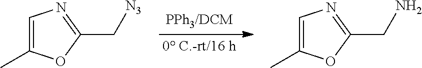

The title compound was synthesized using an analogous procedure used for building block B8 step b) using 2-(azidomethyl)-5-methyloxazole as obtained in step f) as starting material. The crude product was obtained as a brown liquid (130 mg, 53%) which was used in the next step without further purification.

LCMS: m/z 113 (M+1).

### h) 1-(((5-methyloxazol-2-yl)methyl)amino)cyclopentanecarbonitrile (B27)

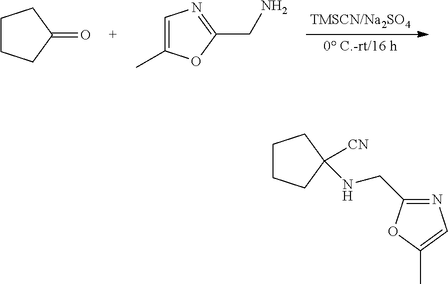

The title compound was synthesized using an analogous procedure used for building block B3 using (5-methyloxazol-2-yl)methanamine as obtained in step g) as the starting material. The crude product was obtained as a brown liquid (203 mg) which was used without further purification.

### Building block B28: 1-(((5-(((tert-butyldimethylsilyl)oxy)methyl)oxazol-2-yl)methyl)amino)cyclopentanecarbonitrile

### a)ethyl 2-(bromomethyl)oxazole-5-carboxylate

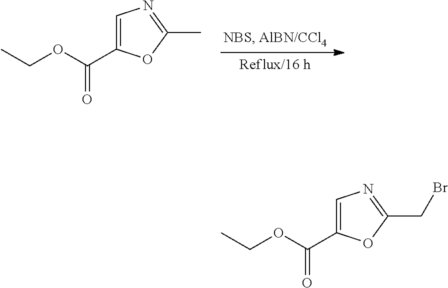

To a stirred solution of ethyl 2-methyloxazole-5-carboxylate (1.0 g, 6.4 mmol) in dry carbon tetrachloride (25 mL) was added NBS (1.4 g, 9.6 mmol), followed by the addition of AlBN (420 mg, 2.5 mmol) and the reaction mixture was refluxed for 16 h. Once the starting material was consumed (monitored by TLC), the reaction mixture was extracted with DCM. The organic phase was washed with water, brine then dried over Na₂SO₄ and concentrated and then purified by column chromatography (silica gel, 10% EtOAc in hexane) provided the title compound (350 mg, 23%).

¹H NMR (400 MHz, DMSO-d₆): δ 8.90 (s, 1H), 4.80 (s, 2H), 4.29 (q, J=6.8 Hz, 2H), 1.28 (t, J=6.9 Hz, 3H); LCMS: m/z 234 (M+1).

### b)ethyl 2-(azidomethyl)oxazole-5-carboxylate

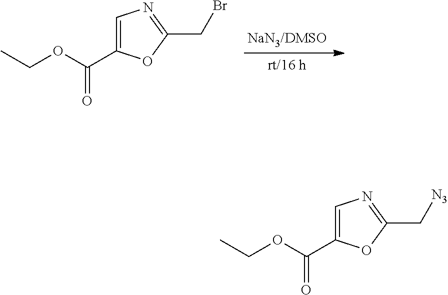

The title compound was synthesized using an analogous procedure to that used for building block B8 step a) using ethyl 2-(bromomethyl)oxazole-5-carboxylate as obtained in step a) as the starting material. The crude compound was obtained as a pale yellow liquid (700 mg) and used in the next step without further purification.

### c) (2-(azidomethyl)oxazol-5-yl)methanol

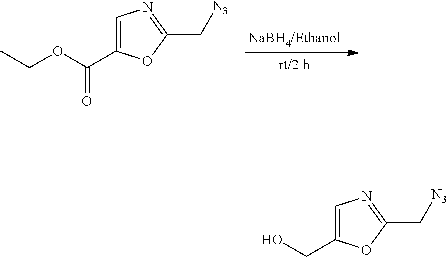

To a stirred solution of ethyl 2-(azidomethyl)oxazole-5-carboxylate (700 mg, 3.5 mmol) in ethanol (15 mL) at room temperature was added sodium borohydride (271 mg, 7.1 mmol) portionwise and the reaction mixture was stirred for 2 h. Once the starting material was consumed (monitored by TLC), the reaction mixture was concentrated and extracted with ethyl acetate. The organic layer was washed with water, brine and dried over sodium sulphate and concentrated to get the desired product (310 mg) as a pale yellow liquid. The crude product was used in the next step without further purification.

¹H NMR (400 MHz, DMSO-d₆): δ 7.96 (s, 1H), 5.20 (t, J=5.8 Hz, 1H), 4.60 (s, 2H), 4.35 (d, J=4.9 Hz, 2H); LCMS: m/z 155 (M+1).

### d) 2-(azidomethyl)-5-(((tert-butyldimethylsilyl)oxy)methyl)oxazole

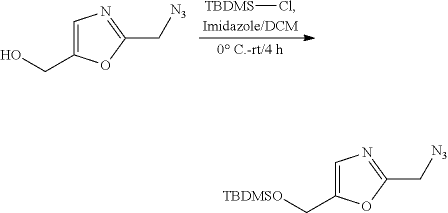

To a stirred solution of (2-(azidomethyl)oxazol-5-yl)methanol (310 mg, 2.0 mmol) as obtained in step c) in DCM (10 mL) at 0° C. was added TBDMS-Cl (455 mg, 3.0 mmol), followed by the addition of imidazole (273 mg, 4.0 mmol). The reaction mixture was stirred at rt for 4 h. Once the starting material was consumed (monitored by TLC), the reaction mixture was extracted with DCM and the organic layer was washed with water, brine then dried over Na₂SO₄ and concentrated and then purified by column chromatography (silica gel, 5% EtOAc in hexane) to provide the title compound (300 mg, 56%) as brown color liquid.

LCMS: m/z 269 (M+1).

### e) (5-(((tert-butyldimethylsilyl)oxy)methyl)oxazol-2-yl)methanamine (R95)

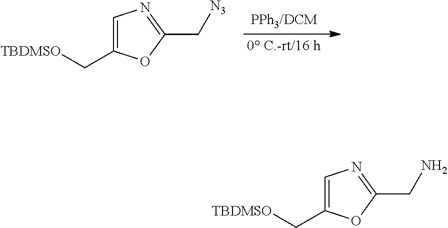 [SMILES: CC(C)(C)[Si](C)(C)OCc1cnc(CN=[N+]=[N-])o1 ; CC(C)(C)[Si](C)(C)OCc1cnc(CN)o1]

The title compound was synthesized using an analogous procedure to that used for building block B8 step b) using 2-(azidomethyl)-5-(((tert-butyldimethylsilyl)oxy)methyl)oxazole as obtained in step d) as the starting material. Product was purified by column chromatography (silica gel, 1% MeOH in DCM) to provide the title compound (90 mg, 33%) as a brown color liquid.

LCMS: m/z 243 (M+1).

### f) 1-(((5-(((tert-butyldimethylsilyl)oxy)methyl)oxazol-2-yl)methyl)amino)cyclopentanecarbonitrile (B28)

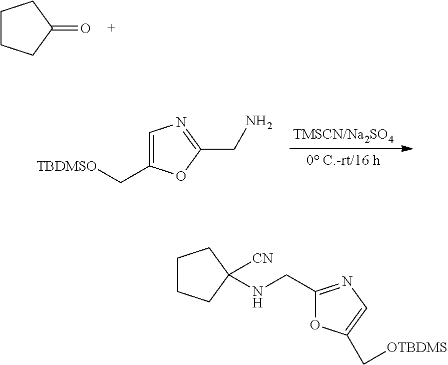 [SMILES: O=C1CCCC1 ; CC(C)(C)[Si](C)(C)OCc1cnc(CN)o1 ; CC(C)(C)[Si](C)(C)OCc1cnc(CNC2(C#N)CCCC2)o1]

The title compound was synthesized using an analogous procedure to that used for building block B3 using (5-(((tert-butyldimethylsilyl)oxy)methyl)oxazol-2-yl)methanamine as obtained in step e) and cyclopentanone as the starting materials. The crude product (124 mg) was used without further purification.

MS (ES): m/z 336 (M+1).

### Building block B29: 1-((oxazol-5-ylmethyl)amino)cyclopentanecarbonitrile

### a) oxazol-5-ylmethanol

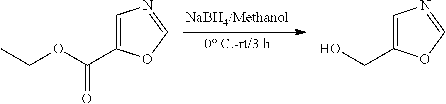

The title compound was synthesized using similar procedure used for building block B27 step d) using ethyl oxazole-5-carboxylate as a starting material.

¹H NMR (400 MHz, DMSO-d₆): δ 8.28 (s, 1H), 7.03 (s, 1H), 5.36 (t, J=5.8 Hz, 1H), 4.46 (d, J=5.9 Hz, 2H).

### b) 5-(bromomethyl)oxazole

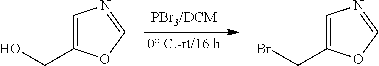 [SMILES: OCc1cnco1 ; BrCc1cnco1]

The title compound was synthesized using the same procedure used for R4 using 2-(bromomethyl)-5-methyloxazole as obtained in step a) as a starting material. The crude product (700 mg) was used in the next step without further purification.

### c) 5-(azidomethyl)oxazole

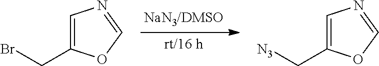 [SMILES: BrCc1cnco1 ; [N-]=[N+]=NCc1cnco1]

The title compound was synthesized using analogous procedure used for building block B8 step a) using 5-(bromomethyl)oxazole as obtained in step b) as a starting material. The crude compound (420 mg) was used in the next step without further purification.

### d) oxazol-5-ylmethanamine

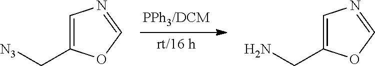 [SMILES: [N-]=[N+]=NCc1cnco1 ; NCc1cnco1]

The title compound was synthesized using an analogous procedure used for building block B8 step b) using 5-(azidomethyl)oxazole as obtained in step c) as a starting material. The crude product (180 mg) was obtained as a pale yellow semi-solid which was used in the next step without further purification.

### e) 1-((oxazol-5-ylmethyl)amino)cyclopentanecarbonitrile (B29)

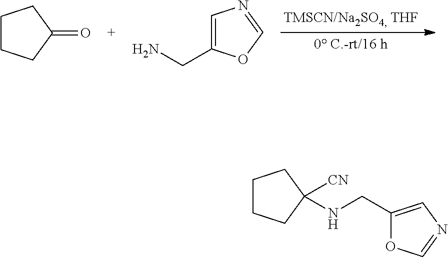 [SMILES: O=C1CCCC1 ; NCc1cnco1 ; N#CC1(NCc2cnco2)CCCC1]

The title compound was synthesized using an analogous procedure to that used for building block B3 using oxazol-5-ylmethanamine as obtained in step d) and cyclopentanone as starting materials. The crude product was obtained as yellow color gummy solid (350 mg) which was used in the next step without further purification.

### Building block B30

### a)ethyl 2-methyloxazole-5-carboxylate

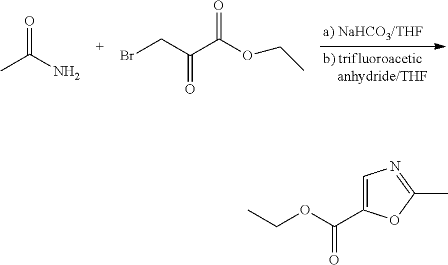

To a stirred solution of acetamide (1.0 g, 16.9 mmol) in THF (15 mL) was added sodium hydrogen carbonate (7.0 g, 83.3 mmol), followed by the addition of ethyl 3-bromo-2-oxopropanoate (5.0 g, 25.0 mmol) at 0° C. The reaction mixture was heated at 85° C. for 16 h. The reaction mixture was cooled to room temperature, filtered through celite pad and concentrated. The residue was dissolved in THF (15 mL), followed by the addition of trifluoroacetic anhydride (20 mL, 140.9 mmol) at 0° C. and the reaction mixture was stirred at rt for 1 h. The reaction mixture was concentrated and extracted with ethyl acetate. The organic layer was washed with sat. NaHCO₃ solution, water, brine and dried over sodium sulphate, concentrated. Purification by column chromatography (silica gel, 20% EtOAc in hexane) provided the title compound (300 mg, 8%).

¹H NMR (400 MHz, DMSO-d₆): δ 8.69 (s, 1H), 4.27 (q, J=6.9 Hz, 2H), 2.51 (s, 3H);

LCMS: m/z 155 (M+1).

### b) (2-methyloxazol-5-yl)methanol

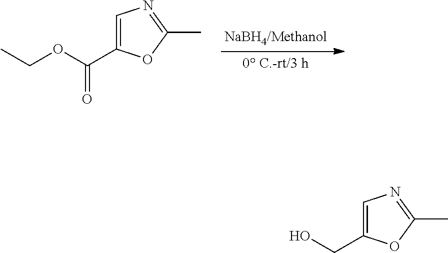

The title compound was synthesized using an analogous procedure to that used for ______ using ethyl 2-methyloxazole-5-carboxylate as obtained in step a) as a starting material.

¹H NMR (400 MHz, DMSO-d₆): δ 7.73 (s, 1H), 5.07 (t, J=5.4 Hz, 1H), 4.29 (d, J=4.4 Hz, 2H), 2.36 (s, 3H); LCMS: m/z 114 (M+1).

### c) 5-(bromomethyl)-2-methyloxazole

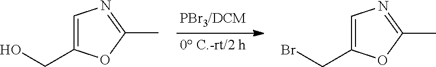

The title compound was synthesized using the same procedure used for building block B27 step d) using 2-(bromomethyl)-5-methyloxazole as a starting material. The crude product (600 mg) was used in the next step without further purification.

### d) 5-(azidomethyl)-2-methyloxazole

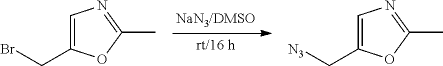

The title compound was synthesized using an analogous procedure to that used for building block B8 step a) using 5-(bromomethyl)-2-methyloxazole as obtained in step c) as a starting material. The crude compound (300 mg) was used in the next step without further purification.

### e) (2-methyloxazol-5-yl)methanamine

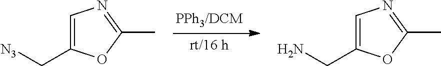

The title compound was synthesized using an analogous procedure used for building block B8 step b) using 5-(azidomethyl)-2-methyloxazole as obtained in step d) as a starting material. The crude product (110 mg) was obtained as a pale yellow semi-solid which was used in the next step without further purification.

MS (ES): m/z 113 (M+1).

### f) 1-(((2-methyloxazol-5-yl)methyl)amino)cyclopentanecarbonitrile (B30)

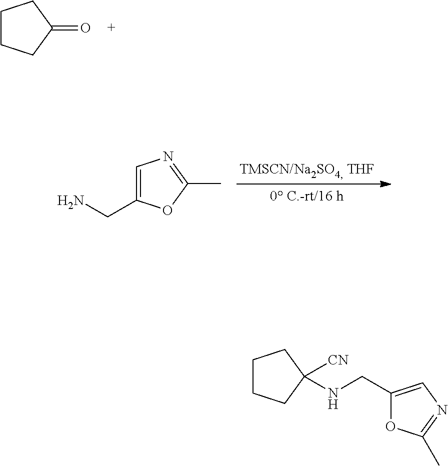

The title compound was synthesized using analogous procedure used for building block B3 using (2-methyloxazole-5-yl)methanamine as obtained in step e) and cyclopentanone as starting materials. The crude product (181 mg) was obtained as yellow color gummy solid which was used without further purification.

### Building block B31: 3-(methylamino)tetrahydrofuran-3-carbonitrile

### a) tetrahydrofuran-3-ol

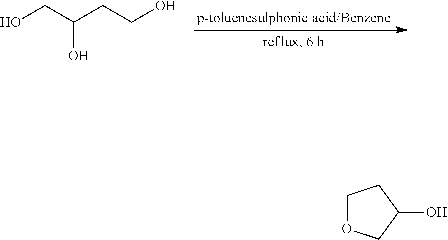 [SMILES: OCCC(O)CO ; OC1CCOC1]

To a stirred solution of butane-1,2,4-triol (1.0 g, 9.0 mmol) in benzene (10 mL) was added p-toluenesulphonic acid (179 mg, 0.9 mmol) and refluxed under Dean-stark apparatus for 6 h. Once the starting material was consumed (monitored by TLC), the reaction mixture was concentrated and purification by column chromatography (silica gel, 4% MeOH in DCM) provided the titled compound (0.3 g, 36%).

¹H NMR (400 MHz, DMSO-d₆): δ 4.79 (d, J=3.4 Hz, 1H), 4.29-4.26 (m, 1H), 3.74-3.62 (m, 3H), 3.47-3.44 (m, 1H), 1.90-1.86 (m, 1H), 1.71-1.70 (m, 1H).

### b) dihydrofuran-3(2H)-one

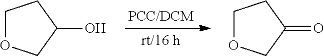 [SMILES: OC1CCOC1 ; O=C1CCOC1]

To a stirred solution of tetrahydrofuran-3-ol (3.0 g, 34 mmol) as obtained in step a) in DCM (60 mL) was added pyridiniumchloro chromate (14.65 g, 68 mmol) at 0° C. and the reaction mixture was stirred at rt for 16 h. Once the starting material was consumed (monitored by TLC), the reaction mixture was filtered through celite and the filtrate was washed with water, brine, drier over anhydrous Na₂SO₄ and concentrated. The crude product (2.5 g) was used in the next step without further purification.

### c) 3-(methylamino)tetrahydrofuran-3-carbonitrile (B31)

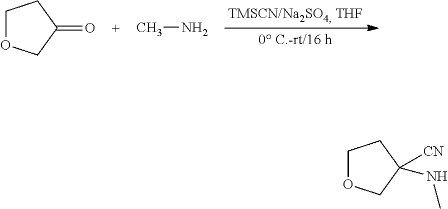

The title compound was synthesized using an analogous procedure to that used for building block B3 using dihydrofuran-3(2H)-one as obtained in step b) and 2M methylamine in THF as the starting material. The crude product (2.2 g) was used without further purification.

### Building block B32: 1-(((tetrahydrofuran-3-yl)methyl)amino)cyclopentanecarbonitrile

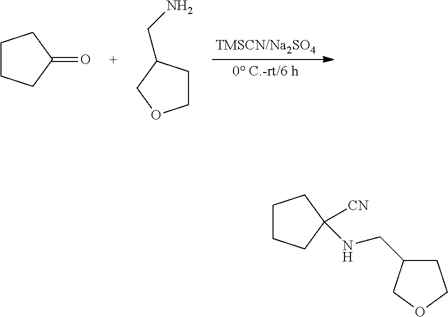 [SMILES: O=C1CCCC1 ; NCC1CCOC1 ; N#CC1(NCC2CCOC2)CCCC1]

The title compound was synthesized using a similar procedure to that used for building block B3 using (tetrahydrofuran-3-yl)methanamine and cyclopentanone as starting materials. The crude product was obtained as a brown liquid (0.15 g) which was used without further purification.

### Building block B33: 1-(methylamino)cyclobutanecarbonitrile

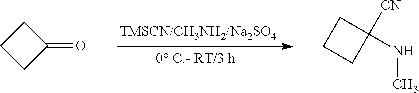

Trimethylsilylcyanide (1.80 mL, 0.014 moles) was added drop wise to a stirred mixture of cyclobutanone (1.0 g, 0.014 moles) in dry tetrahydrofuran (15 mL), 2M methylamine solution in tetrahydrofuran (7.13 mL, 0.014 moles) and sodium sulphate (10.1 g, 0.07 moles) at 0° C. The reaction mixture was allowed to come to room temperature and stirred for 2 h. Once the starting material was consumed (monitored by TLC), the reaction mixture was filtered to remove sodium sulphate. Filtrate was diluted with ethyl acetate. Organic layer was washed with brine solution, dried over Na₂SO₄ and concentrated under reduced pressure to get the crude product as pale brown liquid (1.6 g) which was used without further purification.

### Example 1.0

### Preparation of

2-chloro-4-((5R,6S)-6-hydroxy-1-methyl-2,4-dioxo-1,3-diazaspiro[4.4]nonan-3-yl)-3-methylbenzonitrile 1.0(i);
2-chloro-4-((5S,6R)-6-hydroxy-1-methyl-2,4-dioxo-1,3-diazaspiro[4.4]nonan-3-yl)-3-methylbenzonitrile 1.0(ii);
2-chloro-4-((5R,6R)-6-hydroxy-1-methyl-2,4-dioxo-1,3-diazaspiro[4.4]nonan-3-yl)-3-methylbenzonitrile 1.0(iii);
2-chloro-4-((5S,6S)-6-hydroxy-1-methyl-2,4-dioxo-1,3-diazaspiro[4.4]nonan-3-yl)-3-methylbenzonitrile 1.0(iv).

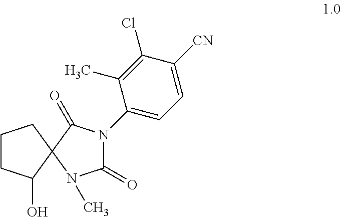 [SMILES: Cc1c(N2C(=O)N(C)C3(CCCC3O)C2=O)ccc(C#N)c1Cl]

### a) 2-chloro-4-(6-hydroxy-1-methyl-4-oxo-2-thioxo-1,3-diazaspiro[4.4]nonan-3-yl)-3-methylbenzonitrile

Triethylamine (4.4 mL, 0.031 moles) was added drop wise to a stirred mixture of 2-(methoxymethoxy)-1-(methylamino)cyclopentanecarbonitrile (Building block B1) (3.9 g, 0.021 moles) in dichloromethane (50 mL) and 2-chloro-4-isothiocyanato-3-methylbenzonitrile (building block A1) (4.4 g, 0.021 moles) at 0° C. Then the reaction mixture was allowed to warm to room temperature and continued stirring for 4 h. Once the starting material disappeared (monitored by TLC), solvent was distilled out from the reaction mixture under reduced pressure. The residue was dissolved in methanol and 2N HCl (40 mL/13 mL). The solution was then heated to reflux and stirred for 4 h at the same temperature. After the reaction mixture was cooled to room temperature, it was poured on crushed ice and extracted with ethyl acetate (3×200 mL). Organic layer was washed with brine solution, dried over Na₂SO₄ and concentrated under reduced pressure. Purification by column chromatography (silica gel, 35% EtOAc in hexane) provided the title compound.

Wt of the product: 1.5 g (20%)

¹H NMR (400 MHz, DMSO-d₆): δ 8.04 and 7.98 (2d, J=8.3 Hz, 1H), 7.58 and 7.51 (2d, J=8.3 Hz, 1H)), 5.93-5.87 (m, 1H), 4.36-4.23 (m, 1H), 3.32 (s, 3H), 2.34-2.23 (m, 1H), 2.20 (s, 3H), 2.18-2.06 (m, 2H), 2.01-1.91 (m, 1H), 1.85-1.75 (m, 1H), 1.72-1.65 (m, 1H);

MS (ES): m/z 349.9 (M+1).

### b) 2-chloro-4-(6-hydroxy-1-methyl-2,4-dioxo-1,3-diazaspiro[4.4]nonan-3-yl)-3-methylbenzonitrile

Ruthenium (111) chloride hydrate (0.45 mg, 0.00021 moles) was added portion wise to a stirred cold mixture of 2-chloro-4-(6-hydroxy-1-methyl-4-oxo-2-thioxo-1,3-diazaspiro[4.4]nonan-3-yl)-3-methylbenzonitrile (1.5 g, 4.3 mmol) and sodium (meta) periodate (1.83 g, 8.6 mmol) in carbon tetrachloride (5 mL), water (10 mL) and acetonitrile (5 mL) at 0° C. The reaction mixture was allowed to stir at room temperature for 3 h. Once the starting material disappeared (monitored by TLC), reaction mixture was quenched with saturated aqueous sodium thiosulphate solution followed by saturated aqueous sodium hydrogen carbonate solution. The reaction mixture was extracted with ethyl acetate (3×100 mL). Organic layer was washed with brine solution, dried over Na₂SO₄ and concentrated under reduced pressure. Purification by column chromatography (silica gel, 35% EtOAc in hexane) provided the title compound as a mixture of four isomers 0.5 g (35%). The isomers were separated by preparative HPLC.

HPLC method: Column: Lux Cellulose-2; Column Dimension: (250×21.1 mm), 5 μm; Mobile phase A: n-hexane; B: EtOH (90:10); Flow Rate: 17.0 ml/min; Wavelength: 210.0 nm.

RT-Isomer 1: 33.589 min; RT-Isomer 2: 36.704 min; RT-Isomer 3: 42.098 min; RT-Isomer 4: 44.818 min.

### Isomer 1:

¹H NMR (400 MHz, CDCl₃): δ 7.61 (d, J=8.3 Hz; 1H), 7.23 (d, J=8.3 Hz; 1H), 4.52-4.46 (m, 1H), 3.16 (s, 3H), 2.35-2.30 (m, 1H), 2.28 (s, 3H), 2.25 (s, 1H), 2.19-2.11 (m, 1H), 2.07-1.90 (m, 2H), 1.90-1.80 (m, 2H);

MS (ES): m/z 334.2 (M+1).

MP: 126° C.

### Isomer 2:

¹H NMR (400 MHz, CDCl₃): δ 7.62 (d, J=8.3 Hz; 1H), 7.24 (d, J=8.3 Hz; 1H), 4.52-4.46 (m, 1H), 3.16 (s, 3H), 2.35-2.30 (m, 1H), 2.28 (s, 3H), 2.25 (s, 1H), 2.19-2.11 (m, 1H), 2.03-2.01 (m, 1H), 1.92-1.80 (m, 3H);

MS (ES): m/z 334.1 (M+1).

MP: 188° C.

Isomers 3 and 4 were not further characterised.

### Example 1.1

### Preparation of 2-chloro-3-methyl-4-(1-methyl-2,4,6-trioxo-1,3-diazaspiro[4.4]nonan-3-yl)benzonitrile 1.1

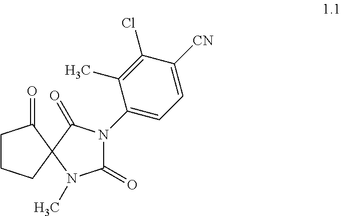 [SMILES: Cc1c(N2C(=O)N(C)C3(CCCC3=O)C2=O)ccc(C#N)c1Cl]

Dessmartin's periodinane (0.430 g, 1 mmol) was added to a cold stirred solution of 2-chloro-4-(6-hydroxy-1-methyl-2,4-dioxo-1,3-diazaspiro[4.4]nonan-3-yl)-3-methylbenzonitrile (1.0) (0.280 g, 0.8 mmol) in dry dichloromethane at 0° C. and the reaction mixture was stirred for 1 h at room temperature. Once the starting material disappeared (monitored by TLC), the reaction mixture was quenched with saturated aqueous Na₂S₂O₃ solution and extracted with EtOAc. Organic layer was washed with water followed by saturated NaHCO₃ solution, dried over anhydrous Na₂SO₄ and concentrated under reduced pressure. Purification of the crude compound by column chromatography (silica gel, 35% EtOAc in hexane) provided the title compound as pale brown colored solid as a mixture of isomers.

Wt of the product: 0.210 g (78%)

¹H NMR (400 MHz, CDCl₃): δ 7.62-7.54 (m, 1H), 7.22-7.15 (t, 1H), 2.89 (s, 3H), 2.71-2.55 (m, 2H), 2.47-2.41 (m, 2H), 2.40-2.32 (m, 1H), 2.30 (s, 3H), 2.23-2.21 (m, 1H);

IR (KBr): 3078, 2961, 2239, 1778, 1718, 1591, 1481, 1402, 1325, 1244, 1124 cm⁻¹;

MS (LC-MS): m/z 330.0 (M−1).

MP: 161° C.

### Example 1.2

### Preparation of 2-chloro-4-(4,6-dihydroxy-1-methyl-2-oxo-1,3-diazaspiro[4.4]nonan-3-yl)-3-methylbenzonitrile 1.2

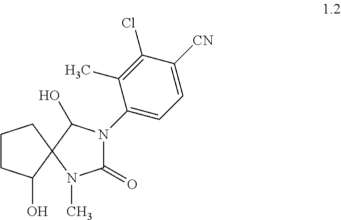 [SMILES: Cc1c(N2C(=O)N(C)C3(CCCC3O)C2O)ccc(C#N)c1Cl]

### a) 4-(6-((tert-butyldimethylsilyl)oxy)-1-methyl-2,4-dioxo-1,3-diazaspiro[4.4]nonan-3-yl)-2-chloro-3-methylbenzonitrile

Tert-butyldimethylsilylchloride (250 mg, 1.7 mmol) was added portion wise to a stirred mixture of 2-chloro-4-(6-hydroxy-1-methyl-2,4-dioxo-1,3-diazaspiro[4.4]nonan-3-yl)-3-methylbenzonitrile (1.0) as a mixture of isomers (0.11 g, 0.33 mmol) and imidazole (0.112 g, 1.7 mmol) in dry dichloromethane (5 mL) at room temperature. The reaction mixture was heated to reflux and continued for 24 h. Once the starting material disappeared (monitored by TLC), reaction mixture was quenched with saturated aqueous sodium-bi-carbonate solution and extracted with dichloromethane (3×15 mL). Organic layer was washed with brine solution, dried over Na₂SO₄ and concentrated under reduced pressure to give the crude product which after purification by column chromatography (silica gel, 15% EtOAc in hexane) provided the title compound.

Wt of the crude product: 0.1 g (68%)

¹H NMR (400 MHz, DMSO-d₆): δ 8.03 and 7.98 (2 d, J=8.3 Hz, 1H), 7.56 and 7.20 (2 d, J=8.4 Hz, 1H), 4.39-4.34 (m, 1H), 3.02 (s, 3H), 2.19 (s, 3H), 1.93-1.91 (m, 2H), 1.76-1.64 (m, 4H), 0.85 (s, 9H), 0.09 (s, 6H);

MS (ES): m/z 448.1 (M+1).

### b) 4-(6-((tert-butyldimethylsilyl)oxy)-4-hydroxy-1-methyl-2-oxo-1,3-diazaspiro[4.4]nonan-3-yl)-2-chloro-3-methylbenzonitrile

Sodium borohydride (250 mg, 6.7 mmol) was added portion wise to a stirred mixture of 4-(6-((tert-butyldimethylsilyl)oxy)-1-methyl-2,4-dioxo-1,3-diazaspiro[4.4]nonan-3-yl)-2-chloro-3-methylbenzonitrile (0.1 g, 0.22 mmol) in methanol (5 mL) at 0° C. The reaction mixture was allowed to warm to room temperature and stirred for 16 h. Once the starting material disappeared (monitored by TLC), reaction mixture was quenched with saturated ammonium chloride solution and extracted with dichloromethane (3×15 mL). Organic layer was washed with brine solution, dried over Na₂SO₄ and concentrated under reduced pressure to get the crude product which was used in the next step without further purification.

Wt of the crude product: 0.075 g (75%)

¹H NMR (400 MHz, DMSO-d₆): δ 7.86 (d, J=8.8 Hz; 1H), 7.51 (d, J=8.8 Hz; 1H), 6.50 (d, J=8.3 Hz; 1H), 4.83 (d, J=8.3 Hz; 1H), 4.13 (t, J=4.9 Hz; 1H), 2.85 (s, 3H), 2.28 (s, 3H), 2.12-2.00 (m, 2H), 1.99-1.88 (m, 1H), 1.83-1.77 (m, 1H), 1.57-1.44 (m, 2H);

MS (ES): m/z 450.3 (M+1).

### c) 2-chloro-4-(4,6-dihydroxy-1-methyl-2-oxo-1,3-diazaspiro[4.4]nonan-3-yl)-3-methylbenzonitrile 1.2

Tetrabutylammonium fluoride (1M solution in THF) (0.78 mL, 0.8 mmol) was added dropwise to a solution of 4-(6-((tert-butyldimethylsilyl)oxy)-4-hydroxy-1-methyl-2-oxo-1,3-diazaspiro[4.4]nonan-3-yl)-2-chloro-3-methylbenzonitrile (35 mg, 0.08 mmol) in dry tetrahydrofuran (2 mL) at 0° C. The reaction mixture was allowed to warm to room temperature and stirred for 3 h. Once the starting material disappeared (monitored by TLC), reaction mixture was quenched with ice and extracted with ethyl acetate (3×5 mL). Organic layer was washed with brine solution, dried over Na₂SO₄ and concentrated under reduced pressure. Purification of the residue by column chromatography (silica gel, 45% EtOAc in hexane) provided the title compound as a mixture of isomers.

Wt of the product: 0.008 g (30%)

¹H NMR (400 MHz, CDCl₃): δ 7.53 (d, J=8.3 Hz; 1H), 7.44-7.37 (dd, J, =8.3 Hz & J₂=8.3 Hz; 1H), {4.97 (bs) and 4.80-4.78 (m), 1H}, 4.49 and 4.12 (2 bs, 1H), 3.02 (s, 3H), 2.75 (bs, 1H), 2.35 (s, 3H), 2.24-2.17 (m, 1H), 2.12-2.04 (m, 2H); 1.97-1.96 (m, 2H), 1.79-1.64 (m, 2H);

MS (ES): m/z 336.1 (M+1);

MP: 179° C.

### Example 1.3

### Preparation of diastereoisomers of 2-chloro-4-(1-ethyl-6-hydroxy-2,4-dioxo-1,3-diazaspiro[4.4]nonan-3-yl)-3-methylbenzonitrile 1.3(i) and 1.3(ii)

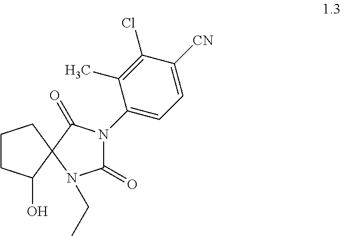 [SMILES: CCN1C(=O)N(c2ccc(C#N)c(Cl)c2C)C(=O)C12CCCC2O]

Triethylamine (0.8 mL, 0.006 moles) was added drop wise to a stirred mixture of 2-(methoxymethoxy)-1-(ethylamino)cyclopentanecarbonitrile (obtained in an analogous way to building block B1) (0.75 g, 3.8 mmol) in dichloromethane (10 mL) and 2-chloro-4-isocyanato-3-methylbenzonitrile (building block A2) (0.73 g, 3.8 mmol) at 0° C. The reaction mixture was stirred at room temperature for 4 h. Once the starting material disappeared (monitored by TLC), the reaction mixture was concentrated under reduced pressure. The residue was dissolved in methanol and 2N HCl (30 mL/10 mL) and heated to reflux for 4 h. The reaction mixture was allowed to cooled to room temperature, poured on crushed ice and extracted with ethyl acetate (3×100 mL). Organic layer was washed with brine solution, dried over Na₂SO₄ and concentrated under reduced pressure. Purification by column chromatography (silica gel, 30% EtOAc in hexane) provided the title compound as a mixture of two isomers and the isomers were separated by HPLC.

Wt of the product: 0.105 g (8%)

### Isomer 1:

Wt of the product: 0.013 g (1%)

¹H NMR (400 MHz, CDCl₃): δ 7.61 (d, J=8.2 Hz; 1H), 7.23 (d, J=8.2 Hz; 1H), 4.46-4.41 (m, 1H), 3.62-3.48 (m, 2H), 2.37-2.30 (m, 1H), 2.28 (s, 3H), 2.25 (s, 1H), 2.21-2.12 (m, 1H), 2.11-2.00 (m, 1H), 1.94-1.78 (m, 3H), 1.41-1.37 (dt, J₁=1.3 Hz & J₂=3.5 Hz; 3H),

MS (ES): m/z 348.1 (M+1).

RT=50.71 min, ee=97.35% [Cellulose-2, solvent A=Hexane, solvent B=IPA (0.1% DEA), solvent C=MeOH, A=210 nm, 90/10 solvent A/solvent B];

MP: 138° C.

### Isomer 2:

Wt of the product: 0.009 g (ca. 1%)

¹H NMR (400 MHz, CDCl₃): δ 7.61 (d, J=8.2 Hz; 1H), 7.24 (d, J=8.2 Hz; 1H), 4.45-4.43 (m, 1H), 3.60-3.49 (m, 1H), 2.37-2.30 (m, 1H), 2.28 (s, 3H), 2.25 (s, 1H), 2.17-2.16 (m, 1H), 2.03-2.01 (m, 1H), 1.94-1.79 (m, 3H), 1.41-1.37 (dt, J₁=3.0 Hz & J₂=7.1 Hz; 3H),

MS (ES): m/z 348.0 (M+1).

RT=59.98 min, ee=98.15% [Cellulose-2, solvent A=Hexane, solvent B=IPA (0.1% DEA), solvent C=MeOH, A=210 nm, 90/10 solvent A/solvent B];

MP: 135° C.

### Example 1.4

### Preparation of

2-chloro-4-((5S,6S)-6-fluoro-1-methyl-2,4-dioxo-1,3-diazaspiro[4.4]nonan-3-yl)-3-methylbenzonitrile 1.4(i);
2-chloro-4-((5R,6R)-6-fluoro-1-methyl-2,4-dioxo-1,3-diazaspiro[4.4]nonan-3-yl)-3-methylbenzonitrile 1.4(ii);
2-chloro-4-((5S,6R)-6-fluoro-1-methyl-2,4-dioxo-1,3-diazaspiro[4.4]nonan-3-yl)-3-methylbenzonitrile 1.4(iii); and
2-chloro-4-((5R,6S)-6-fluoro-1-methyl-2,4-dioxo-1,3-diazaspiro[4.4]nonan-3-yl)-3-methylbenzonitrile 1.4(iv).

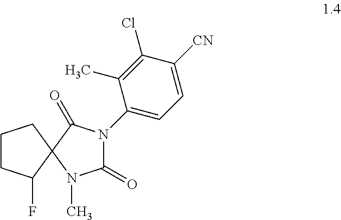 [SMILES: Cc1c(N2C(=O)N(C)C3(CCCC3F)C2=O)ccc(C#N)c1Cl]

Diethyl amino sulphur trifluoride (DAST) (0.012 mL, 0.09 mmol) was added dropwise to a cold stirred solution of each isomer obtained in example 1.0 respectively (0.020 g, 0.06 mmol) in dry dichloromethane (2 mL) at −78° C. The reaction mixture was allowed to come to room temperature and stirred for 3 h. Once the starting material disappeared (monitored by TLC), reaction mixture was quenched with saturated aqueous sodium hydrogen carbonate solution and extracted with dichloromethane (2×5 mL). Organic layer was washed with brine solution, dried over Na₂SO₄ and concentrated under reduced pressure. Purification by column chromatography (silica gel, 35% EtOAc in hexane) provided the title compound.

Isomer 1 (from Isomer 1 of Example 1.0):

Wt of the product: 0.007 g (23%)

¹H NMR (400 MHz, CDCl₃): δ 7.61 (d, J=8.3 Hz; 1H), 7.22 (d, J=8.4 Hz; 1H), 5.25-5.08 (m, 1H), 3.04 (s, 3H), 2.45-2.37 (m, 1H), 2.28 (s, 3H), 2.25-2.22 (m, 1H); 2.21-2.16 (m, 2H), 2.10-2.01 (m, 1H), 1.85-1.81 (m, 1H);

IR (NEAT): 2955, 2237, 1780, 1722, 1479, 1402, 1172, 1132, 1080 cm⁻¹;

MS (ES): m/z 336.2 (M+1);

RT=49.84 min, ee=97.26% [cellulose-2, solvent A=Hexane, solvent B=EtOH, solvent C=MeOH, A=210 nm, 90/10 solvent A/solvent B];

MP: 209° C.

Isomer 2 (from Isomer 2 of Example 1.0):

Wt of the product: 0.008 g (38%)

¹H NMR (400 MHz, CDCl₃): δ 7.61 (d, J=8.3 Hz; 1H), 7.22 (d, J=8.4 Hz; 1H), 5.25-5.08 (m, 1H), 3.04 (s, 3H), 2.46-2.38 (m, 1H), 2.28 (s, 3H), 2.25-2.22 (m, 1H); 2.21-2.16 (m, 2H), 2.09-2.01 (m, 1H), 1.86-1.81 (m, 1H);

IR (NEAT): 2957, 2235, 1780, 1722, 1479, 1402, 1130, 1032, 1011 cm⁻¹;

MS (ES): m/z 336.1 (M+1);

RT=58.95 min, ee=86.89% [cellulose-2, solvent A=Hexane, solvent B=EtOH, solvent C=MeOH, A=210 nm, 90/10 solvent A/solvent B];

MP: 200° C.

Isomer 3 (from Isomer 3 of Example 1.0):

Wt of the products: 0.010 g (41%)

¹H NMR (400 MHz, CDCl₃): δ 7.61 (d, J=8.3 Hz; 1H), 7.22 (d, J=8.3 Hz; 1H), {[5.23 (t, J=8.8 Hz), 5.10 (t, J=8.8 Hz); 1H}, 3.04 (s, 3H), 2.47-2.37 (m, 1H), 2.28 (s, 3H), 2.23-2.22 (m, 1H); 2.21-2.17 (m, 2H), 2.11-2.01 (m, 1H), 1.84-1.81 (m, 1H);

IR (NEAT): 2956, 2237, 1780, 1722, 1479, 1402, 1173, 1134, 1080 cm⁻¹;

MS (ES): m/z 336.1 (M+1);

RT=48.91 min, ee=99.05% [cellulose-2, solvent A=Hexane, solvent B=EtOH, solvent C=MeOH, A=210 nm, 90/10 solvent A/solvent B];

MP: 211° C.

Isomer 4 (from Isomer 4 of Example 1.0):

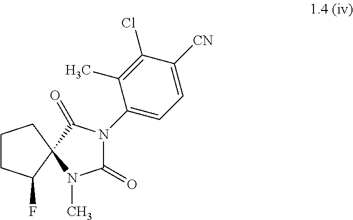 [SMILES: Cc1c(N2C(=O)N(C)[C@@]3(CCC[C@@H]3F)C2=O)ccc(C#N)c1Cl]

Wt of the product: 0.009 g (39%)

¹H NMR (400 MHz, CDCl₃): δ 7.61 (d, J=8.3 Hz; 1H), 7.22 (d, J=8.3 Hz; 1H), [5.23 (t, J=8.5 Hz), 5.10 (t, J=9.0 Hz); 1H], 3.03 (s, 1H), 2.45-2.37 (m, 1H), 2.28 (s, 3H), 2.24-2.22 (m, 1H); 2.21-2.17 (m, 2H), 2.08-2.01 (m, 1H), 1.84-1.81 (m, 1H);

IR (NEAT): 2961, 2237, 1780, 1722, 1479, 1402, 1171, 1132, 1082 cm⁻¹;

MS (ES): m/z 336.2 (M+1);

RT=56.27 min, ee=93.08% [cellulose-2, solvent A=Hexane, solvent B=EtOH, solvent C=MeOH, A=210 nm, 90/10 solvent A/solvent B];

MP: 204° C.

The absolute configuration of isomer 4 was confirmed by X-ray crystal structure as being 2-chloro-4-((5R,6S)-6-fluoro-1-methyl-2,4-dioxo-1,3-diaza-spiro[4.4]non-3-yl)-3-methylbenzonitrile.

### Example 1.5

### Preparation of

(S)-2-chloro-3-methyl-4-(1-methyl-2,4-dioxo-1,3-diazaspiro[4.4]non-6-en-3-yl)benzonitrile 1.5(i); and
(R)-2-chloro-3-methyl-4-(1-methyl-2,4-dioxo-1,3-diazaspiro[4.4]non-6-en-3-yl)benzonitrile 1.5(ii).

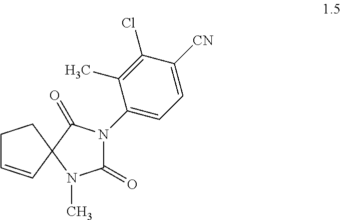 [SMILES: Cc1c(N2C(=O)N(C)C3(C=CCC3)C2=O)ccc(C#N)c1Cl]

Diethyl amino sulphur trifluoride (DAST) (0.012 mL, 0.09 mmol) was added dropwise to a cold stirred solution of each of isomers 3 and 4 of example 1.0 (0.020 g, 0.06 mmol) in dry dichloromethane (2 mL) at −78° C. The reaction mixture was allowed to come to room temperature and stirred for 3 h. Once the starting material disappeared (monitored by TLC), reaction mixture was quenched with saturated aqueous sodium hydrogen carbonate solution and extracted with dichloromethane (2×5 mL). Organic layer was washed with brine solution, dried over Na₂SO₄ and concentrated under reduced pressure. Purification by column chromatography (silica gel, 35% EtOAc in hexane) provided the title compound.

Isomer 1 (from Isomer 3 of Example 1.0):

Wt of the product: 0.003 g (13%).

¹H NMR (400 MHz, CDCl₃): δ 7.63 (d, J=8.3 Hz; 1H), 7.23 (d, J=8.3 Hz; 1H), 6.42 (d, J=3.5 Hz; 1H), 5.54-5.50 (m, 1H), 2.90 (s, 3H), 2.70-2.66 (m, 2H), 2.56-2.51 (m, 1H), 2.27 (s, 3H), 2.21-2.15 (m, 1H),

IR (NEAT): 2924, 2235, 1776, 1719, 1479, 1398, 1161, 1134 cm⁻¹;

MS (ES): m/z 316.1 (M+1);

RT=60.25 min, ee=98.38% [cellulose-2, solvent A=Hexane, solvent B=EtOH, solvent C=MeOH, A=210 nm, 90/10 solvent A/solvent B];

MP: 181° C.

Isomer 2 (from Isomer 4 of Example 1.0):

Wt of the product: 0.002 g (9%).

¹H NMR (400 MHz, CDCl₃): δ 7.63 (d, J=8.3 Hz; 1H), 7.23 (d, J=8.3 Hz; 1H), 6.42-6.41 (dd, J, =1.5 Hz & J₂; 3.9 Hz; 1H), 5.54-5.50 (m, 1H), 2.90 (s, 3H), 2.73-2.67 (m, 2H), 2.66-2.51 (m, 1H), 2.27 (s, 3H), 2.21-2.15 (m, 1H),

IR (NEAT): 2924, 2235, 1776, 1720, 1479, 1398, 1275, 1132 cm⁻¹;

MS (ES): m/z 316.2 (M+1);

RT=66.42 min, ee=93.22% [cellulose-2, solvent A=Hexane, solvent B=EtOH, solvent C=MeOH, A=210 nm, 90/10 solvent A/solvent B];

MP: 180° C.

### Example 1.6

### Preparation of 2-chloro-4-(6-(hydroxymethyl)-1-methyl-2,4-dioxo-1,3-diazaspiro[4.4]nonan-3-yl)-3-methylbenzonitrile 1.6

### a) 2-chloro-4-(6-((methoxymethoxy)methyl)-2,4-dioxo-1,3-diazaspiro[4.4]nonan-3-yl)-3-methylbenzonitrile

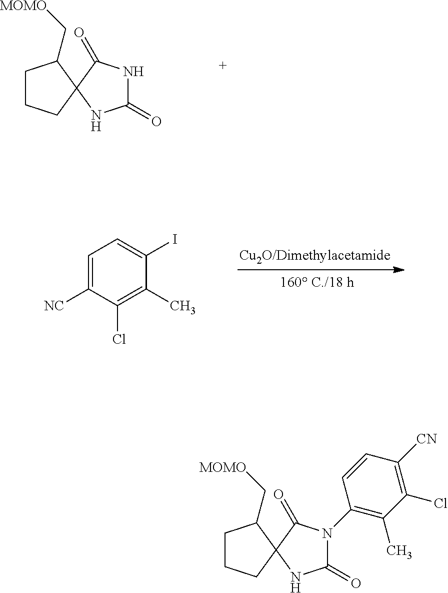

Cuprous oxide (1.7 g, 0.01 moles) was added to stirred solution of 6-((methoxymethoxy)methyl)-1,3-diazaspiro[4.4]nonane-2,4-dione (building block B15) (1.4 g, 0.006 moles) in dimethylacetamide (5 mL) and 2-chloro-4-iodo-3-methylbenzonitrile (1.7 g, 0.006 moles) at room temperature. The reaction mixture was heated to 160° C. and stirred for 18 h. Once the starting material was consumed (monitored by TLC), the reaction mixture was diluted with water and extracted with ethyl acetate (2×100 mL). The organic layer was washed with water, dried over anhydrous Na₂SO₄ and concentrated under reduced pressure. Purification by column chromatography (silica gel, 40% EtOAc in hexane) provided the title compound as pale yellow solid.

MS (LC-MS): m/z 378.1 (M+1);

### b) 2-chloro-4-(6-((methoxymethoxy)methyl)-1-methyl-2,4-dioxo-1,3-diazaspiro[4.4]nonan-3-yl)-3-methylbenzonitrile

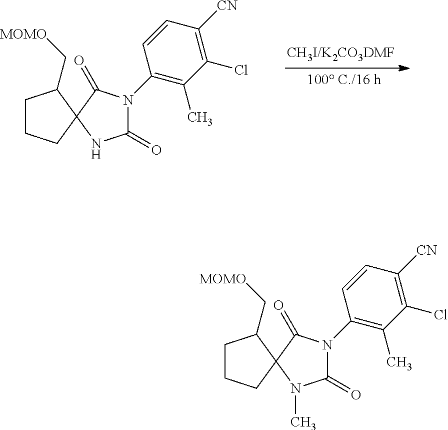

To a stirred solution of 2-chloro-4-(6-((methoxymethoxy)methyl)-2,4-dioxo-1,3-diazaspiro[4.4]nonan-3-yl)-3-methylbenzonitrile as obtained in step a) (0.85 g, 0.002 moles) in DMF (10 mL) was added potassium carbonate (0.94 g, 0.007 moles) followed by methyl iodide (0.3 mL, 0.004 moles) in a sealed tube at room temperature. The reaction mixture was heated to 100° C. and stirred for 16 h at the same temperature. Once the starting material was consumed (monitored by TLC), the reaction mixture was diluted with water and extracted with ethyl acetate (2×100 mL). The organic layer was washed with water, dried over anhydrous Na₂SO₄ and concentrated under reduced pressure. Purification by column chromatography (silica gel, 50% EtOAc in hexane) provided the title compound as pale yellow solid (0.3 g, 33%).

MS (LC-MS): m/z 392.2 (M+1).

### c) 2-chloro-4-(6-(hydroxymethyl)-1-methyl-2,4-dioxo-1,3-diazaspiro[4.4]nonan-3-yl)-3-methylbenzonitrile

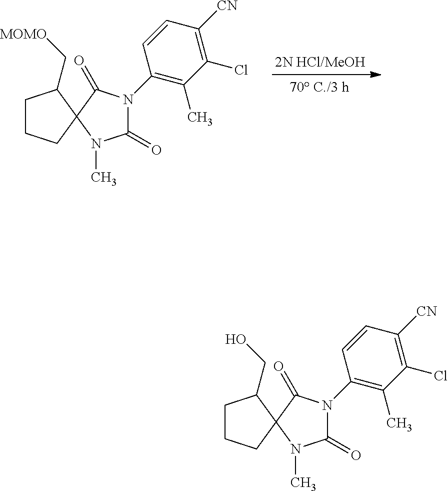

2N Hydrochloric acid (3.0 mL) was added to a stirred solution of 2-chloro-4-(6-((methoxymethoxy)methyl)-1-methyl-2,4-dioxo-1,3-diazaspiro[4.4]nonan-3-yl)-3-methylbenzonitrile as obtained in step b) (0.3 g, 0.007 moles) in methanol (3 mL) at room temperature. The reaction mixture was heated to 70° C. and stirred for 3 h at the same temperature. Once the starting material was consumed (monitored by TLC), the reaction mixture was concentrated under reduced pressure. The residue was diluted with water and extracted with ethyl acetate (2×10 mL). The organic layer was washed with water, dried over anhydrous Na₂SO₄ and concentrated under reduced pressure. Purification by column chromatography (silica gel, 50% EtOAc in hexane) provided the title compound as white solid (0.19 g, 71%).

¹H NMR (400 MHz, CDCl₃): δ 7.57 (d, J=7.6 Hz; 1H), 7.20 (d, J=7.6 Hz, 1H), 3.91-3.90 (m, 2H), 3.63-3.58 (m, 1H), 3.12-3.00 (3s, 3H), 2.34-2.27 (3s, 3H), 2.16-2.00 (m, 6H);

MS (LC-MS): m/z 348.1 (M+1);

MP: 157° C.

### Example 1.7

### Preparation of 2-chloro-4-(1,6-dimethyl-2,4-dioxo-1,3-diazaspiro[4.4]nonan-3-yl)-3-methylbenzonitrile 1.7

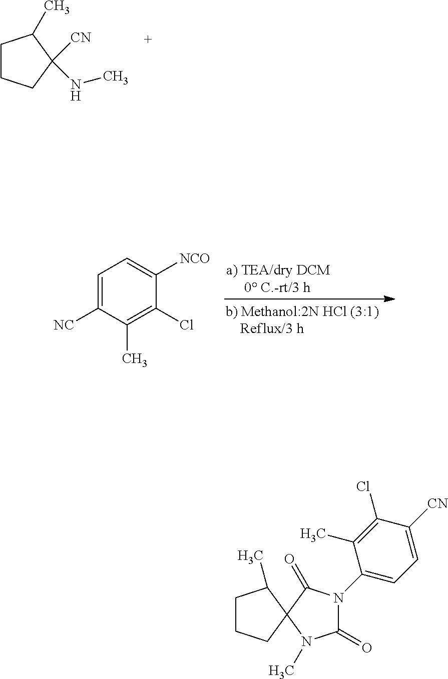

The title compound was synthesized using an analogous procedure used for example 8.1 using 2-methyl-1-(methylamino)cyclopentanecarbonitrile (building block B26) and 2-chloro-4-isocyanato-3-methylbenzonitrile (building block A2) as the starting materials. Purification by column chromatography (silica gel, 40% EtOAc in hexane) provided the title compound as a mixture of four isomers (0.54 g, 46%). The isomers were separated by preparative HPLC.

HPLC method: Column: Lux Amylose-2; Column Dimension: (250×21.2 mm); 5 μm;

Mobile phase A: n-hexane; B: IPA (70:30); Flow Rate: 17.0 ml/min; Wavelength: 241.0 nm.

RT-Isomer 1: 25.66 min; RT-Isomer 2: 27.97 min; RT-Isomer 3: 28.67 min; RT-Isomer 4: 34.15 min.

### Isomer 1:

Weight of the product: 10 mg (5%)

¹H NMR (400 MHz, DMSO): δ 7.99-7.96 (m, 1H), 7.58-7.38 (m, 1H), 2.89 (d, J=3.4 Hz; 3H), 2.39-2.32 (m, 3H), 2.20 (s, 3H), 2.11-2.07 (m, 1H), 1.93-1.77 (m, 4H), 1.64-1.62 (m, 1H), 0.94 (t, J=6.9 Hz; 3H);

MS (LC): m/z 332.1 (M+1).

Chiral HPLC: RT=12.90 min, ee=98.67% [Chiralcel OD-H, solvent A=Hexane, solvent B=IPA, solvent C=MeOH, A=241 nm, 80/20 solvent A/solvent B];

### Isomer 2:

Weight of the product: 10 mg (5%)

¹H NMR (400 MHz, DMSO): δ 7.99-7.96 (m, 1H), 7.58-7.38 (m, 1H), 2.89 (s, 3H), 2.39-2.32 (m, 1H), 2.21 (s, 3H), 2.13-2.11 (m, 1H), 1.99-1.77 (m, 4H), 1.64-1.59 (m, 1H), 0.94 (t, J=6.9 Hz; 3H);

MS (LC): m/z 332.1 (M+1).

Chiral HPLC: RT=15.58 min, ee=99.95% [Chiralcel OD-H, solvent A=Hexane, solvent B=IPA, solvent C=MeOH, A=215 nm, 80/20 solvent A/solvent B];

### Isomer 3:

Weight of the product: 20 mg (10%)

¹H NMR (400 MHz, DMSO): δ 7.99-7.96 (m, 1H), 7.58-7.49 (m, 1H), 2.98 (d, J=2.9 Hz; 3H), 2.39-2.31 (m, 1H), 2.20 (s, 3H), 2.16-2.14 (m, 2H), 2.06-1.96 (m, 2H), 1.78-1.62 (m, 1H), 1.59-1.54 (m, 1H), 0.99 (t, J=6.8 Hz; 3H);

MS (LC): m/z 332.1 (M+1).

Chiral HPLC: RT=26.67 min, ee=99.37% [Lux Amylose-2, solvent A=Hexane, solvent B=IPA, solvent C=MeOH, A=241 nm, 70/30 solvent A/solvent B];

MP: 140° C.

### Isomer 4:

Weight of the product: 20 mg (10%)

¹H NMR (400 MHz, DMSO): δ 7.99-7.96 (m, 1H), 7.58-7.49 (m, 1H), 2.97 (d, J=3.0 Hz; 3H), 2.39-2.33 (m, 1H), 2.23-2.18 (m, 2H), 2.20 (s, 3H), 2.06-2.01 (m, 2H), 1.99-1.77 (m, 1H), 1.62-1.54 (m, 1H), 0.99 (t, J=6.8 Hz; 3H);

MS (LC): m/z 332.1 (M+1).

Chiral HPLC: RT=31.39 min, ee=96.63% [Lux Amylose-2, solvent A=Hexane, solvent B=IPA, solvent C=MeOH, A=241 nm, 70/30 solvent A/solvent B];

MP: 164° C.

### Example 2.0

### Preparation of two isomers of 2-chloro-4-(6-hydroxy-1-methyl-2-oxo-1,3-diazaspiro[4.4]nonan-3-yl)-3-methylbenzonitrile 2.0(i) and 2.0(ii)

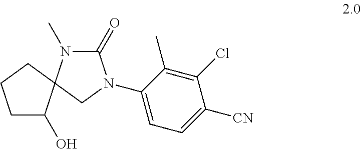 [SMILES: Cc1c(N2CC3(CCCC3O)N(C)C2=O)ccc(C#N)c1Cl]

### a) tert-butyl(2-((tert-butyldimethylsilyl)oxy)-1-(((3-chloro-4-cyano-2-methylphenyl)amino)-methyl)cyclopentyl)carbamate

To a stirred solution of tert-butyl(2-((tert-butyldimethylsilyl)oxy)-1-formylcyclopentyl)carbamate (building block B2) (1.4 g, 0.004 moles) in MeOH (20 mL) were added 4-amino-2-chloro-3-methylbenzonitrile (0.67 g, 0.004 moles) and AcOH (6 mL) at 0° C. and the reaction mixture was stirred for 15 minutes then slowly allowed to warm to room temperature and stirred for an additional 3 h. It was then cooled to 0° C., and to it was added NaCNBH₃ (0.33 g, 4.8 mmol) and the reaction mixture was stirred for 16 h. Once the starting material disappeared (monitored by TLC), the reaction mixture was quenched with ammonium chloride and extracted with ethyl acetate (3×30 mL) and the organic layer was washed with water, brine solution, dried over Na₂SO₄ and concentrated under reduced pressure. Purification by column chromatography (silica gel, 7% EtOAc in hexane) provided the title compound.

Wt of the product: 0.62 g (31%)

¹H NMR (400 MHz, CDCl₃): δ 7.36 (d, J=8.8 Hz, 1H), 6.41 (s, 1H), 6.30 (d, J=8.8 Hz, 1H), 5.47 (s, 1H), 3.90 (t, J=7.4 Hz, 1H), 3.69 (s, 1H), 3.25 (m, 1H), 3.10 (m, 1H), 2.70 (m, 1H), 2.24 (s, 3H), 2.00-1.55 (m, 6H), 1.43 (s, 9H), 0.93 (s, 9H), 0.12 (s, 6H).

MS (ES): m/z 494 [M+1].

### b) tert-butyl(1-(((3-chloro-4-cyano-2-methylphenyl)amino)methyl)-2-hydroxycyclopentyl)-carbamate

To a stirred solution of tert-butyl(2-((tert-butyldimethylsilyl)oxy)-1-(((3-chloro-4-cyano-2-methylphenyl)amino)methyl)cyclopentyl)carbamate (0.42 g, 0.008 moles) in THF (15 mL) was added TBAF (1.27 mL, 0.001 moles) at 0° C. stirred for 30 minutes. Once the starting material disappeared (monitored by TLC), the reaction mixture was diluted with water and extracted with ethyl acetate (3×20 ml). The organic layer was washed with water, brine solution, dried over Na₂SO₄ and concentrated under reduced pressure. Purification of crude by column chromatography (silica gel, 25% EtOAc in hexane) provided the title compound.

Wt of the product: 0.29 g (90%) MS (ES): m/z 378 [M−1].

### c) 2-((tert-butoxycarbonyl)amino)-2-(((3-chloro-4-cyano-2-methylphenyl)amino)methyl)-cyclopentyl acetate

To a stirred solution of tert-butyl(1-(((3-chloro-4-cyano-2-methylphenyl)amino)methyl)-2-hydroxycyclopentyl)carbamate (0.29 g, 0.007 moles) in DCM (12 mL) was added acetic anhydride (0.07 mL, 0.007 moles), TEA (0.08 mL, 0.007 moles) at 0° C. and catalytic amount of DMAP was added, stirred for 3 h at room temperature. Once the starting material disappeared (monitored by TLC), the reaction mixture was diluted with water and extracted with ethyl acetate. The organic layer was washed with water, brine solution, dried over Na₂SO₄ and concentrated under reduced pressure. Purification of crude by column chromatography (silica gel, 10% EtOAc in hexane) provided the title compound.

Wt of the product: 0.33 g (90%)

¹H NMR (400 MHz, CDCl₃): δ 7.35 (d, J=8.4 Hz, 1H), 6.44 (d, J=8.8 Hz, 1H), 5.96 (s, 1H), 5.12 (t, J=7.8 Hz, 1H), 4.98 (s, 1H), 3.49-3.21 (m, 2H), 2.66 (s, 1H), 2.24 (s, 3H), 2.14 (s, 3H), 1.88-1.55 (m, 6H), 1.45 (s, 9H);

MS (ES): m/z 420 [M−1].

### d) 2-amino-2-(((3-chloro-4-cyano-2-methylphenyl)amino)methyl)cyclopentyl acetate

To a stirred solution of 2-((tert-butoxycarbonyl)amino)-2-(((3-chloro-4-cyano-2-methylphenyl)-amino)methyl)cyclopentyl acetate (0.33 g, 0.007 moles) in DCM (20 mL) was added TFA (0.9 mL, 11 mmol) at 0° C. and stirring continued for 3 h at room temperature. Once the starting material disappeared (monitored by TLC), the reaction mixture was basified with sodium bicarbonate and extracted with ethyl acetate. The organic layer with washed with water, brine solution, dried over Na₂SO₄ and concentrated under reduced pressure to get the crude product which was used in the next step without further purification.

Wt of the product: 0.18 g (71%)

MS (ES): m/z 320 [M−1].

### e) 3-(3-chloro-4-cyano-2-methylphenyl)-2-oxo-1,3-diazaspiro[4.4]nonan-6-yl acetate

To a stirred solution of 2-amino-2-(((3-chloro-4-cyano-2-methylphenyl)amino)methyl)cyclopentyl acetate (0.18 g, 0.5 mmol) in dry THF (10 mL) were added COCl₂ (0.328 mL, 0.6 mmol) and DIPEA (0.15 mL, 0.8 mmol) at 0° C. and the reaction mixture was stirred for 30 minutes at room temperature. Once the starting material disappeared (monitored by TLC), the reaction mixture was diluted with water and extracted with ethyl acetate. The organic layer was washed with water, brine solution, dried over Na₂SO₄ and concentrated under reduced pressure. The crude reaction mixture was washed with ether to provide the title compound.

Wt of the crude product: 0.16 g (82%)

¹H NMR (400 MHz, DMSO): δ 7.83 (d, J=8.3 Hz, 1H), 7.60 (s, 1H), 7.44 (d, J=8.3 Hz, 1H), 5.01-4.99 (m, 1H), 3.79-3.66 (m, 2H), 2.27 (s, 3H), 2.06 (s, 3H), 2.14 (s, 3H), 1.88-1.55 (m, 6H);

MS (ES): m/z 348 [M+1].

### f) 3-(3-chloro-4-cyano-2-methylphenyl)-1-methyl-2-oxo-1,3-diazaspiro[4.4]nonan-6-yl acetate

To a stirred solution of 3-(3-chloro-4-cyano-2-methylphenyl)-2-oxo-1,3-diazaspiro[4.4]nonan-6-yl acetate (0.16 g, 0.4 mmol) in dry THF (10 mL) was added NaH (0.018 g, 0.4 mmol) followed by MeI (0.029 mL, 0.4 mmol) at 0° C. and the reaction mixture was stirred for 30 minutes at room temperature. Once the starting material disappeared (monitored by TLC), the reaction mixture was diluted with water and extracted with ethyl acetate and washed with water, brine solution, dried over Na₂SO₄ and concentrated under reduced pressure. Purification of crude by column chromatography (silica gel, 10% EtOAc in hexane) provided the title compound.

Wt of the crude product: 0.07 g (42%)

¹H NMR (400 MHz, CDCl₃): δ 7.51 (d, J=8.3 Hz, 1H), 7.18 (d, J=8.3 Hz, 1H), 5.16-5.13 (m, 1H), 3.65-3.56 (m, 2H), 2.96 (s, 3H), 2.34 (s, 3H), 2.10 (s, 3H), 2.03-1.63 (m, 6H);

MS (ES): m/z 362 [M+1].

### a) 2-chloro-4-(6-hydroxy-1-methyl-2-oxo-1,3-diazaspiro[4.4]nonan-3-yl)-3-methylbenzonitrile 2.0

To a stirred solution of 3-(3-chloro-4-cyano-2-methylphenyl)-1-methyl-2-oxo-1,3-diazaspiro[4.4]nonan-6-yl acetate (0.07 g, 0.2 mmol) in MeOH (5 mL) was added K₂CO₃ (0.053 g, 0.3 mmol) and the reaction mixture was stirred for 2 h at room temperature. Once the starting material disappeared (monitored by TLC), MeOH was removed under reduced pressure and the reaction mixture was diluted with water and extracted with ethyl acetate, washed with water, brine solution, dried over Na₂SO₄ and concentrated under reduced pressure. The crude reaction mixture was washed with ether to provide the title compound as a mixture of isomers.

Wt of the product: 0.04 g (65%)

The isomers were separated by preparative HPLC (column: Lux Cellulose-2 (250×4.6 mm) 5 μm and Mobile phase: A: n-Hexane:B: 0.1% TFA in Ethanol in the ratio of 50:50 with the flow rate of 0.8 mL/min, wavelength at 282 nm.)

### Isomer 1

RT: 8.16 mins

### Isomer 2

RT: 9.93 mins

For the purification, the same method was used on each isomer using preparative column Lux Cellulose-2 (250×21.2 mm) 5 μm with a flow rate of 16 mL/min.

NMR data was similar for both isomers:

¹H NMR (400 MHz, CDCl₃): δ 7.51 (d, J=8.3 Hz, 1H), 7.19 (d, J=8.8 Hz, 1H), 4.10 (s, 1H), 3.56-3.39 (m, 2H), 3.03 (s, 3H), 2.34 (s, 3H), 2.08-1.61 (m, 6H);

MS (ES): m/z 320 [M+1].

### Example 3.0

### Preparation of 2-chloro-3-methyl-4-(1-methyl-2,4-dioxo-1,3-diazaspiro[4.4]nonan-3-yl)benzonitrile 3.0

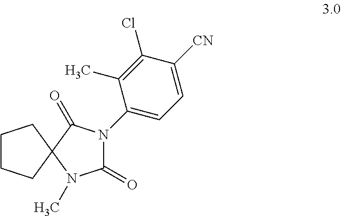 [SMILES: Cc1c(N2C(=O)N(C)C3(CCCC3)C2=O)ccc(C#N)c1Cl]

### a) 2-chloro-3-methyl-4-(1-methyl-4-oxo-2-thioxo-1,3-diazaspiro[4.4]nonan-3-yl)benzonitrile

The title compound was synthesized using a procedure analogous to example 1.0, step a.

Wt of the product: 1.3 g (32%)

¹H NMR (400 MHz, CDCl₃): δ 7.64 (d, J=8.3 Hz, 1H), 7.24 (d, J=8.3 Hz, 1H), 3.32 (s, 3H), 2.27-2.26 (m, 1H), 2.23 (s, 3H), 2.21-2.19 (m, 1H), 2.09-1.92 (m, 6H);

MS (ES): m/z 349.9 (M+1).

### b) 2-chloro-3-methyl-4-(1-methyl-2,4-dioxo-1,3-diazaspiro[4.4]nonan-3-yl)benzonitrile 3.0

The title compound was synthesized using a procedure analogous to example 1.0, step b.

Wt of the product: 0.48 g (39%)

¹H NMR (400 MHz, CDCl₃): δ 7.62 (d, J=7.8 Hz, 1H), 7.23 (d, J=7.8 Hz, 1H), 3.0 (s, 3H), 2.26 (s, 3H), 2.21-2.15 (m, 2H), 2.05-1.89 (m, 6H);

IR (KBr): 3102, 2965, 2235, 1769, 1713, 1591, 1479, 1322, 1277, 1150 cm⁻¹;

MS (ES): m/z 318.1 (M+1);

MP: 154° C.

### Example 3.1

### Preparation of 2-chloro-4-(1-(2-methoxyethyl)-2,4-dioxo-1,3-diazaspiro[4.4]nonan-3-yl)-3-methylbenzonitrile 3.1

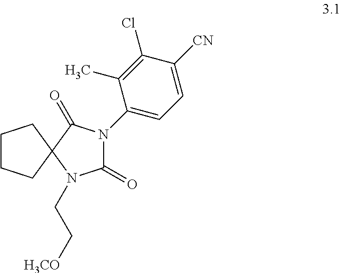 [SMILES: COCCN1C(=O)N(c2ccc(C#N)c(Cl)c2C)C(=O)C12CCCC2]

### a) 2-chloro-4-(1-(2-methoxyethyl)-4-oxo-2-thioxo-1,3-diazaspiro[4.4]nonan-3-yl)-3-methylbenzonitrile

The title compound was synthesized using a procedure analogous to example 1.0, step a, using building block B4.

Wt of the product: 1.1 g (54%)

¹H NMR (400 MHz, CDCl₃): δ 7.64 (d, J=8.3 Hz, 1H), 7.25 (d, J=8.3 Hz, 1H), 3.90-3.78 (m, 4H), 3.38 (s, 3H), 2.23 (s, 3H), 2.21-2.17 (m, 4H), 1.99-1.93 (m, 4H);

MS (ES): m/z 378.1 (M+1).

### b) 2-chloro-4-(1-(2-methoxyethyl)-2,4-dioxo-1,3-diazaspiro[4.4]nonan-3-yl)-3-methylbenzonitrile 3.1

The title compound was synthesized using a procedure analogous to example 1.0, step b.

Wt of the product: 0.25 g (24%)

¹H NMR (400 MHz, CDCl₃): δ 7.62 (d, J=8.3 Hz, 1H), 7.25 (d, J=8.3 Hz, 1H), 3.67-3.64 (m, 2H), 3.51-3.47 (m, 2H), 3.38 (s, 3H), 2.26 (s, 3H), 2.19-2.04 (m, 4H), 1.96-1.88 (m, 4H);

IR (NEAT): 2936, 2235, 1773, 1717, 1591, 1479, 1409, 1163, 1117 cm⁻¹;

MS (ES): m/z 362.0 (M+1);

MP: 115° C.

### Example 3.2

### Preparation of 2-chloro-4-(1-ethyl-2,4-dioxo-1,3-diazaspiro[4.4]nonan-3-yl)-3-methylbenzonitrile 3.2

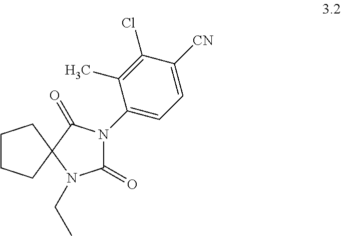 [SMILES: CCN1C(=O)N(c2ccc(C#N)c(Cl)c2C)C(=O)C12CCCC2]

### a) 2-chloro-4-(1-ethyl-4-oxo-2-thioxo-1,3-diazaspiro[4.4]nonan-3-yl)-3-methylbenzonitrile

The title compound was synthesized using a procedure analogous to example 1.0, step a, using building block B5.

Wt of the product: 1.0 g (49%)

¹H NMR (400 MHz, CDCl₃): δ 7.90-7.60 (dd, J, =10.2 Hz & J₂=13.5 Hz; 1H), 7.54 (d, J=8.3 Hz, 1H), 4.11 (s, 3H), 3.81-3.75 (m, 1H), 2.38 (s, 3H), 2.24-2.02 (m, 5H), 1.99-1.82 (m, 2H), 1.45 (t, J=7.1 Hz, 1H);

MS (ES): m/z 348.1 (M+1).

### b) 2-chloro-4-(1-ethyl-2,4-dioxo-1,3-diazaspiro[4.4]nonan-3-yl)-3-methylbenzonitrile 3.2

The title compound was synthesized using a procedure analogous to example 1.0, step b.

Wt of the product: 0.05 g (26%)

¹H NMR (400 MHz, CDCl₃): δ 7.62 (d, J=8.3 Hz, 1H), 7.25 (d, J=8.3 Hz, 1H), 3.44-3.39 (m, 2H), 2.26 (s, 3H), 2.23-2.16 (m, 2H), 2.01-1.97 (m, 4H), 1.95-1.90 (m, 2H), 1.35 (t, J=7.4 Hz, 3H);

MS (ES): m/z 332.1 (M+1);

MP: 94° C.

### Example 4.0

### Preparation of

2-chloro-4-((5S,6R)-6-hydroxy-2,4-dioxo-1-oxa-3-azaspiro[4.4]nonan-3-yl)-3-methylbenzonitrile 4.0(i);
2-chloro-4-((5R,6S)-6-hydroxy-2,4-dioxo-1-oxa-3-azaspiro[4.4]nonan-3-yl)-3-methylbenzonitrile 4.0(ii);
2-chloro-4-((5S,6S)-6-hydroxy-2,4-dioxo-1-oxa-3-azaspiro[4.4]nonan-3-yl)-3-methylbenzonitrile 4.0(iii); and
2-chloro-4-((5R,6R)-6-hydroxy-2,4-dioxo-1-oxa-3-azaspiro[4.4]nonan-3-yl)-3-methylbenzonitrile 4.0(iv).

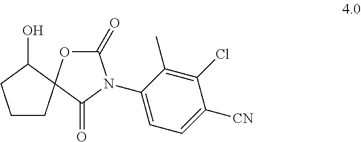 [SMILES: Cc1c(N2C(=O)OC3(CCCC3O)C2=O)ccc(C#N)c1Cl]

### a) 1-cyano-2-(methoxymethoxy)cyclopentyl(3-chloro-4-cyano-2-methylphenyl)carbamate

To a stirred solution of 1-hydroxy-2-(methoxymethoxy)cyclopentanecarbonitrile (building block B7) (0.38 g, 2.2 mmol) in dry dichloromethane (5 ml) was added 2-chloro-4-isocyanato-3-methylbenzonitrile (building block A2) (0.504 g, 2.2 mmol) in dry dichloromethane (5 ml). Triethylamine (0.62 mL, 4.4 mmol) was added dropwise at 0° C. The reaction mixture was allowed to stir at room temperature for 14 h. Once the starting material disappeared (monitored by TLC), the reaction mixture was filtered through celite pad and washed with dichloromethane. The reaction mixture was then concentrated under reduced pressure. Purification by column chromatography (silica gel, 60% EtOAc in hexane) provided the title compound.

Weight of the product: 0.33 g (41%)

¹H NMR (400 MHz, CDCl₃): δ 7.69-7.65 (m, 1H), 7.36-7.28 (m, 1H), 4.72-4.63 (m, 2H), 4.31-4.27 (m, 1H), 3.33 (s, 3H), 2.43-2.19 (m, 9H);

MS (ES): m/z 363.9 (M+1).

### b) 2-chloro-4-(6-hydroxy-2,4-dioxo-1-oxa-3-azaspiro[4.4]nonan-3-yl)-3-methylbenzonitrile 4.0

To a stirred solution of 1-cyano-2-(methoxymethoxy)cyclopentyl(3-chloro-4-cyano-2-methylphenyl)carbamate (0.33 g, 0.9 mmol), in methanol (5 mL) was added 2N HCl (2 mL) at room temperature and then it was refluxed for 1 h. Once the starting material disappeared (monitored by TLC), the reaction mixture was diluted with ethyl acetate, water and extracted. Organic layer was washed with brine solution, dried over Na₂SO₄ and concentrated under reduced pressure to get the crude product which was purified by column chromatography (silica gel, 20% EtOAc in hexane) to provide the title compound as a mixture of four isomers 0.06 g (17%). The isomers were separated by preparative HPLC.

Weight of the product: 0.06 g (17%) (mixture)

NMR and MP data for the mixture:

¹H NMR (400 MHz, DMSO): δ 8.04 (d, J=8.3 Hz, 1H), 7.71 (d, J=8.3 Hz, 1H), 5.95 (d, J=6.8 Hz, 1H), 4.16-4.18 (m, 1H), 2.24 (s, 3H), 2.03-1.99 (m, 2H), 1.83-1.70 (m, 4H).

MS (ES): m/z 319.1 (M−1);

MP: 150° C.

HPLC method: Column: Phenomenex cellulose-2; solvent A=Hexane, solvent B=IPA (0.1% TFA); A=210 nm; 85/15 solvent A/solvent B; flow rate: 1.0 mL/min.

### Isomer 1:

¹H NMR (400 MHz, DMSO): δ 8.05 (dd, J=8.3, 5.1 Hz, 1H), 7.72-7.45 (m, 1H), 5.96-5.94 (m, 1H), 4.20-4.18 (m, 1H), 2.33-2.19 (m, 5H), 2.03-1.99 (m, 1H), 1.85-1.67 (m, 3H);

MS (ES): m/z 319.1 (M−1);

RT=27.90 min, ee=99.20%;

MP: 148° C.

### Isomer 2:

¹H NMR (400 MHz, DMSO): δ 8.05 (dd, J=8.3, 5.1 Hz, 1H), 7.72-7.45 (m, 1H), 5.96-5.94 (m, 1H), 4.19-4.17 (m, 1H), 2.33-2.21 (m, 5H), 2.03-2.02 (m, 1H), 1.85-1.67 (m, 3H);

MS (ES): m/z 319.1 (M−1);

RT=20.89 min, ee=99.22%;

MP: 144° C.

### Isomer 3:

¹H NMR (400 MHz, CDCl₃): δ 7.65 (d, J=7.9 Hz, 1H), 7.28 (d, J=6.4 Hz, 1H), 4.54-4.53 (m, 1H), 2.44-2.42 (m, 1H), 2.34 (s, 2H), 2.30 (s, 1H), 2.26-2.20 (m, 2H), 2.17-1.99 (m, 3H);

MS (ES): m/z 319.3 (M−1);

RT=36.14 min, ee=96.91%;

### Isomer 4:

¹H NMR (400 MHz, CDCl₃): δ 7.65 (d, J=8.4 Hz, 1H), 7.28 (d, J=5.8 Hz, 1H), 4.56-4.51 (m, 1H), 2.45-2.41 (m, 1H), 2.34-2.20 (m, 5H), 2.17-1.99 (m, 3H);

MS (ES): m/z 319.1 (M−1);

RT=43.13 min, ee=98.32%;

MP: 108° C.

### Example 4.1

### Preparation of 2-chloro-4-(6-hydroxy-4-methoxy-2-oxo-1-oxa-3-azaspiro[4.4]nonan-3-yl)-3-methylbenzonitrile 4.1

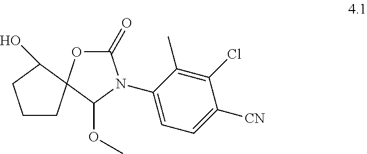 [SMILES: COC1N(c2ccc(C#N)c(Cl)c2C)C(=O)OC12CCCC2O]

### a) 4-(6-((tert-butyldimethylsilyl)oxy)-2,4-dioxo-1-oxa-3-azaspiro[4.4]nonan-3-yl)-2-chloro-3-methylbenzonitrile

To a stirred solution of 2-chloro-4-(6-hydroxy-2,4-dioxo-1-oxa-3-azaspiro[4.4]nonan-3-yl)-3-methylbenzonitrile (4.0) (0.5 g, 1.56 mmol), in dry dichloromethane (10 mL), was added imidazole (0.425 g, 6.25 mmol) and tert-butyldimethylchlorosilane (0.585 g, 3.9 mmol) at 0° C. and stirring was continued at room temperature for 12 h. Once the starting material disappeared (monitored by TLC), the reaction mixture was diluted with dichloromethane, water and extracted. Organic layer was washed with brine solution, dried over Na₂SO₄ and concentrated under reduced pressure to get the crude product which was purified by column chromatography (silica gel, 20% EtOAc in hexane).

Weight of the product: 0.355 g (53%).

¹H NMR (400 MHz, CDCl₃): δ 7.66-7.63 (m, 1H), 7.22-7.20 (m, 1H), 4.51-4.41 (m, 1H), 2.42-2.23 (m, 3H), 2.19-1.87 (m, 6H), 0.88 (s, 9H), 0.10 (s, 6H).

### b) 4-(6-((tert-butyldimethylsilyl)oxy)-4-hydroxy-2-oxo-1-oxa-3-azaspiro[4.4]nonan-3-yl)-2-chloro-3-methylbenzonitrile

To a stirred solution of compound 4-(6-((tert-butyldimethylsilyl)oxy)-2,4-dioxo-1-oxa-3-azaspiro[4.4]nonan-3-yl)-2-chloro-3-methylbenzonitrile (0.1 g, 0.23 mmol), in methanol (5 mL), was added sodium borohydride (0.043 g, 1.15 mmol) slowly at 0° C. and the stirring was continued at room temperature for 1 h. Once the starting material disappeared (monitored by TLC), the reaction mixture was diluted with ethyl acetate, water and extracted. Organic layer was washed with brine solution, dried over Na₂SO₄ and concentrated under reduced pressure to get the crude product which was purified by column chromatography (silica gel, 20% EtOAc in hexane).

Weight of the product: 0.064 g (64%).

¹H NMR (400 MHz, CDCl₃): δ 7.95 (d, J=8.3 Hz, 1H), 7.46 (d, J=8.3 Hz, 1H), 6.81 (d, J=7.3 Hz, 1H), 5.30 (d, J=7.4 Hz, 1H), 4.20 (t, J=6.9 Hz, 1H), 2.31 (s, 3H), 1.65-1.38 (m, 6H), 0.83 (s, 9H), 0.10 (s, 6H).

MS (ES): m/z 437.2 (M+1).

### c) 4-(6-((tert-butyldimethylsilyl)oxy)-4-methoxy-2-oxo-1-oxa-3-azaspiro[4.4]nonan-3-yl)-2-chloro-3-methylbenzonitrile

To a stirred solution of compound 4-(6-((tert-butyldimethylsilyl)oxy)-4-hydroxy-2-oxo-1-oxa-3-azaspiro[4.4]nonan-3-yl)-2-chloro-3-methylbenzonitrile (0.07 g, 0.16 mmol), in dry tetrahydrofuran (3 mL), was added methyliodide (0.015 ml, 0.24 mmol) at 0° C. and then sodiumhydride (0.016 g, 0.4 mmol) was added portion wise. The stirring was continued at room temperature for 1 h. Once the starting material disappeared (monitored by TLC), reaction mixture was diluted with ethyl acetate, water and extracted. Organic layer was washed with brine solution, dried over Na₂SO₄ and concentrated under reduced pressure to get the crude product which was purified by column chromatography (silica gel, 20% EtOAc in hexane).

Weight of the product: 0.020 g (28%).

¹H NMR (400 MHz, CDCl₃): δ 7.57 (d, J=8.3 Hz, 1H), 7.34 (d, J=8.3 Hz, 1H), 4.99 (s, 1H), 4.07 (t, J=7.8 Hz, 1H), 3.18 (s, 3H), 2.44 (s, 3H), 2.17-1.86 (m, 5H), 0.89 (s, 9H), 0.14-0.10 (m, 6H);

MS (ES): m/z 451.3 (M+1).

### d) 2-chloro-4-(6-hydroxy-4-methoxy-2-oxo-1-oxa-3-azaspiro[4.4]nonan-3-yl)-3-methylbenzonitrile 4.1

To a stirred solution of compound 4-(6-((tert-butyldimethylsilyl)oxy)-4-methoxy-2-oxo-1-oxa-3-azaspiro[4.4]nonan-3-yl)-2-chloro-3-methylbenzonitrile (0.02 g, 0.044 mmol), in tetrahydrofuran (2 mL), was added 3N HCl (0.5 mL) at 0° C. and then continued at room temperature for 14 h. Once the starting material disappeared (monitored by TLC), the reaction mixture was diluted with ethyl acetate, water and extracted. Organic layer was washed with brine solution, dried over Na₂SO₄ and concentrated under reduced pressure to get the crude product which was purified by column chromatography (silica gel, 30% EtOAc in hexane).

Weight of the product: 0.005 g (36%).

¹H NMR (400 MHz, DMSO): δ 7.95 (d, J=8.3 Hz, 1H), 7.59 (d, J=8.3 Hz, 1H), 5.45 (d, J=5.8 Hz, 1H), 5.24 (s, 1H), 4.07 (q, 1H), 3.18 (s, 3H), 2.08 (s, 1H), 1.94-1.83 (m, 2H), 1.75-1.59 (m, 3H);

MS (ES): m/z 337.1 (M+1);

IR: 3410, 2951, 2236, 1746, 1719, 1425 cm⁻¹.

### Example 5.0

### Preparation of (S)-2-chloro-3-methyl-4-(2-oxo-1-oxa-3-azaspiro[4.4]non-6-en-3-yl)benzonitrile 5.0(i) and (R)-2-chloro-3-methyl-4-(2-oxo-1-oxa-3-azaspiro[4.4]non-6-en-3-yl)benzonitrile 5.0(ii)

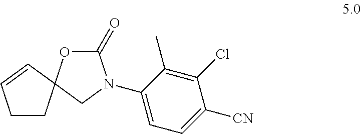 [SMILES: Cc1c(N2CC3(C=CCC3)OC2=O)ccc(C#N)c1Cl]

### a) 4-(6-((tert-butyldimethylsilyl)oxy)-4-hydroxy-2-oxo-1-oxa-3-azaspiro[4.4]nonan-3-yl)-2-chloro-3-methylbenzonitrile

To a stirred solution of 4-(6-((tert-butyldimethylsilyl)oxy)-2,4-dioxo-1-oxa-3-azaspiro[4.4]nonan-3-yl)-2-chloro-3-methylbenzonitrile (0.02 g, 0.046 mmol) in tetrahydrofuran (3 mL), was added lithiumtriethylborohydride (0.1 mL, 0.12 mmol) at −78° C. The reaction mixture was stirred at −78° C. for 3 h. Once the starting material disappeared (monitored by TLC), the reaction mixture was quenched with saturated sodium carbonate solution (0.83 mL). It was allowed to warm to 0° C. and a solution of 30% hydrogen peroxide (0.083 mL) was added dropwise and stirring was continued for 30 min at the same temperature. The reaction mixture was diluted with dichloromethane (50 mL), and the organic layer was washed with brine solution, dried over Na₂SO₄ and concentrated under reduced pressure to get the crude product which was directly used for the next step.

Weight of the product: 0.018 g (90%).

¹H NMR (400 MHz, DMSO): δ 7.95 (d, J=8.3 Hz, 1H), 7.46 (d, J=8.8 Hz, 1H), 6.81 (d, J=7.9 Hz, 1H), 5.30 (d, J=7.8 Hz, 1H), 4.20 (t, J=7.3 Hz, 1H), 2.31 (s, 3H), 1.65-1.35 (m, 6H), 0.83 (s, 9H), 0.10-0.07 (m, 6H);

MS (ES): m/z 437.4 (M+1).

### b) 2-chloro-4-(6-hydroxy-2-oxo-1-oxa-3-azaspiro[4.4]nonan-3-yl)-3-methylbenzonitrile

4-(6-((tert-butyldimethylsilyl)oxy)-4-hydroxy-2-oxo-1-oxa-3-azaspiro[4.4]nonan-3-yl)-2-chloro-3-methylbenzonitrile (0.018 g, 0.041 mmol) was dissolved in dry dichloromethane (2 mL), and triethylsilane (0.08 mL, 0.5 mmol) was added to it at −78° C. followed by addition of borontrifluoride-diethyletherate (0.08 mL, 0.63 mmol). After 2 h at −78° C. an additional amount of triethylsilane (0.08 mL, 0.5 mmol) and borontrifluoridediethyletherate (0.08 mL, 0.63 mmol) were added and stirring was continued for 14 h at 0° C. The reaction mixture was quenched with saturated sodium carbonate solution and then diluted with dichloromethane. Organic layer was washed with brine solution, dried over Na₂SO₄ and concentrated under reduced pressure to get the crude product which was purified by column chromatography (silica gel, 30% EtOAc in hexane) to give the title compound as a mixture of four isomers 0.111 g (38%).

Weight of the product: 0.076 g (26%) (mixture)

The following NMR, MS and IR data refer to the mixture of isomers.

¹H NMR (400 MHz, DMSO): δ 7.91 (d, J=7.8 Hz, 1H), 7.57 (d, J=8.8 Hz, 1H), 5.34 (d, J=5.8 Hz, 1H), 4.08-4.06 (m, 1H), 3.85-3.83 (m, 2H), 2.29 (s, 3H), 2.08-2.06 (m, 1H) 1.90-1.85 (m, 2H), 1.63-1.59 (m, 3H).

MS (ES): m/z 307.1 (M+1).

IR: 3294, 2922, 2236, 1742, 1589, 1487 cm⁻¹.

The isomers were separated by preparative HPLC.

HPLC method: Column: Lux cellulose-2; solvent A=Hexane, solvent B=EtOH; A=260 nm; 60/40 solvent A/solvent B, flow rate: 0.8 mL/min.

### Isomer 1:

¹H NMR (400 MHz, DMSO): δ 7.91 (d, J=8.8 Hz, 1H), 7.57 (d, J=8.8 Hz, 1H), 5.34 (d, J=6.3 Hz, 1H), 4.08-4.06 (m, 1H), 3.85-3.83 (m, 2H), 2.29 (s, 3H), 2.08-2.04 (m, 1H), 1.93-1.85 (m, 2H), 1.63-1.59 (m, 3H);

MS (ES): m/z 307.1 (M+1);

RT=9.12 min, ee=99.56%;

MP: 157° C.

### Isomer 2:

¹H NMR (400 MHz, DMSO): δ 7.91 (d, J=8.8 Hz, 1H), 7.57 (d, J=8.3 Hz, 1H), 5.35 (d, J=5.8 Hz, 1H), 4.08-4.06 (m, 1H), 3.85-3.83 (m, 2H), 2.29 (s, 3H), 2.08-2.04 (m, 1H), 1.93-1.85 (m, 2H), 1.63-1.59 (m, 3H);

MS (ES): m/z 307.1 (M+1);

RT=12.74 min, ee=99.28%;

MP: 157° C.

The other two isomers could not be isolated as pure products.

### c) 2-chloro-3-methyl-4-(2-oxo-1-oxa-3-azaspiro[4.4]non-6-en-3-yl)benzonitrile 5.0

### Isomer 1

To a stirred solution of 2-chloro-4-(6-hydroxy-2-oxo-1-oxa-3-azaspiro[4.4]nonan-3-yl)-3-methylbenzonitrile (isomer 1 obtained in step b) (0.015 g, 0.046 mmoles), in dry dichloromethane (2 mL), was added diethylaminosulphurtrifluoride (0.01 mL, 0.076 mmol) at 0° C. and then continued at room temperature for 1 h. Once the starting material disappeared (monitored by TLC), the reaction mixture was diluted with dichloromethane, water and extracted. Organic layer was washed with brine solution, dried over Na₂SO₄ and concentrated under reduced pressure to get the crude product which was purified by column chromatography (silica gel, 30% EtOAc in hexane).

Weight of the product: 0.004 g (28%).

¹H NMR (400 MHz, CDCl₃): δ 7.57 (d, J=8.3 Hz, 1H), 7.28 (d, J=8.8 Hz, 1H), 6.26-6.24 (m, 1H), 5.89-5.87 (m, 1H), 2.70-2.65 (m, 1H), 2.52-2.48 (m, 3H), 2.39 (s, 3H), 2.21-2.17 (m, 2H).

MS (ES): m/z 289.2 (M+1).

IR: 3451, 2972, 2928, 2232, 1753, 1589, 1479 cm⁻¹. Isomer 2

To a stirred solution of 2-chloro-4-(6-hydroxy-2-oxo-1-oxa-3-azaspiro[4.4]nonan-3-yl)-3-methylbenzonitrile (isomer 2 obtained in step b) (0.015 g, 0.000046 moles) in dry dichloromethane (2 ml), was added diethylaminosulphurtrifluoride (0.01 ml) at 0° C. and the reaction mixture was stirred at room temperature for 1 h. Once the starting material disappeared (monitored by TLC), reaction mixture was diluted with dichloromethane, water and extracted. Organic layer was washed with brine solution, dried over Na₂SO₄ and concentrated under reduced pressure to get the crude product which was purified by column chromatography (silica gel, 30% EtOAc in hexane).

Weight of the product: 0.004 g (28%).

¹H NMR (400 MHz, CDCl₃): δ 7.57 (d, J=8.3 Hz, 1H), 7.28 (d, J=8.3 Hz, 1H), 6.26-6.24 (m, 1H), 5.89-5.87 (m, 1H), 2.70-2.65 (m, 1H), 2.55-2.48 (m, 3H), 2.39 (s, 3H), 2.21-2.16 (m, 2H).

MS (ES): m/z 288.9 (M+1).

IR: 2930, 2855, 2234, 1753, 1589, 1479 cm⁻¹.

### Example 6.0

### Preparation of 2-chloro-4-(7-hydroxy-1-methyl-2,4-dioxo-1,3-diazaspiro[4.4]nonan-3-yl)-3-methylbenzonitrile 6.0

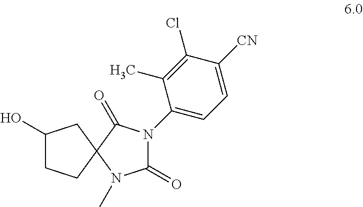 [SMILES: Cc1c(N2C(=O)N(C)C3(CCC(O)C3)C2=O)ccc(C#N)c1Cl]

The title compound was synthesized using a procedure analogous to example 1.2 using 3-(methoxymethoxy)-1-(methylamino)cyclopentanecarbonitrile (building block B6) and 2-chloro-4-isocyanato-3-methylbenzonitrile (building block A2).

Wt of the product: 0.150 g (8%)

¹H NMR (400 MHz, DMSO-d₆): δ 7.99-7.97 (dd, J₁=8.3 Hz & J₂=1.5 Hz; 1H), 7.58-7.54 (dd, J₁=8.3 Hz & J₂=7.8 Hz; 1H), 4.99-4.97 (m, 1H), 4.30 (s, 1H), 2.96 (s, 3H), 2.33-2.22 (m, 1H), 2.20-2.18 (m, 3H), 2.10-2.00 (m, 1H), 1.98-1.88 (m, 2H), 1.83-1.75 (m, 2H);

MS (ES): m/z 333.9 (M+1).

### Example 6.1

### Preparation of two isomers of 2-chloro-4-(7-fluoro-1-methyl-2,4-dioxo-1,3-diazaspiro[4.4]nonan-3-yl)-3-methylbenzonitrile 6.1(i) and 6.1(ii)

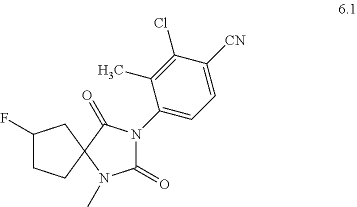 [SMILES: Cc1c(N2C(=O)N(C)C3(CCC(F)C3)C2=O)ccc(C#N)c1Cl]

The title compound was obtained using a procedure analogous to that of example 2.1, starting with compound 6.0. Two isomeric products were separated by HPLC method.

HPLC method: Chiral pak AD-H, solvent A=Hexane, solvent B=EtOH, (A:B=50:50) (sample prepared in MeOH+Mobile Phase and sonicated), A=210 nm, 50/50 solvent A/solvent B.

### Isomer 1:

¹H NMR (400 MHz, CDCl₃): δ 7.63 (d, J=8.4 Hz; 1H), 7.25-7.22 (dd, J, =2.5 Hz & J₂=5.9 Hz; 1H), 5.43-5.30 (m, 1H), 2.98 (s, 3H), 2.62-2.52 (m, 2H), 2.42-2.30 (m, 2H), 2.26 (s, 3H), 2.14-2.06 (m, 2H);

IR (KBr): 2920, 2235, 1771, 1715, 1591, 1479, 1406, 1134 cm⁻¹;

MS (ES): m/z 336.3 (M+1);

RT=15.87 min, ee=99.81%;

MP: 158° C.

### Isomer 2:

¹H NMR (400 MHz, CDCl₃): δ 7.63 (d, J=7.9 Hz; 1H), 7.25-7.22 (dd, J, =3.1 Hz & J₂=5.9 Hz; 1H), 5.43-5.30 (m, 1H), 2.98 (s, 3H), 2.62-2.52 (m, 2H), 2.42-2.31 (m, 2H), 2.26 (s, 3H), 2.11-2.06 (m, 2H);

IR (KBr): 2924, 2234, 1778, 1720, 1591, 1479, 1402, 1136 cm⁻¹;

MS (ES): m/z 336.1 (M+1);

RT=22.64 min, ee=99.49%;

MP: 162° C.

### Example 6.2

### Preparation of 2-chloro-3-methyl-4-(1-methyl-2,4,7-trioxo-1,3-diazaspiro[4.4]nonan-3-yl)benzonitrile 6.2

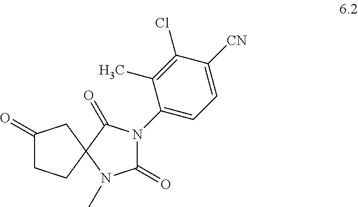 [SMILES: Cc1c(N2C(=O)N(C)C3(CCC(=O)C3)C2=O)ccc(C#N)c1Cl]

Dessmartin's periodinane (DMP) (0.150 g, 0.37 mmol) was added to a cold stirred solution of 2-chloro-4-(7-hydroxy-1-methyl-2,4-dioxo-1,3-diazaspiro[4.4]nonan-3-yl)-3-methylbenzonitrile (6.1) (0.05 g, 0.15 mmol) in dry dichloromethane at 0° C. and the reaction mixture was stirred for 12 h at room temperature. Once the starting material disappeared (monitored by TLC), dichloromethane was evaporated and the residue was diluted with EtOAc, water and extracted. Organic layer was washed with water followed by saturated NaHCO₃ solution, dried over anhydrous Na₂SO₄ and concentrated under reduced pressure. Purification of the crude compound by column chromatography (silica gel, 40% EtOAc in hexane) provided the title compound as cream colored solid.

Wt of the product: 0.042 g (84%)

¹H NMR (400 MHz, CDCl₃): δ 7.64 (d, J=7.8 Hz; 1H), 7.25 (d, J=7.8 Hz; 1H), 3.04 (s, 3H), 2.84-2.76 (m, 2H), 2.64-2.57 (m, 3H), 2.44-2.42 (m, 2H), 2.29-2.24 (m, 2H);

MS (ES): m/z 332.2 (M+1);

MP: 219° C.

### Example 6.3

### Preparation of 2-chloro-4-(7,7-difluoro-1-methyl-2,4-dioxo-1,3-diazaspiro[4.4]nonan-3-yl)-3-methylbenzonitrile 6.3

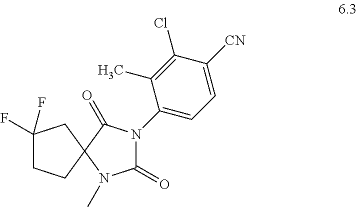 [SMILES: Cc1c(N2C(=O)N(C)C3(CCC(F)(F)C3)C2=O)ccc(C#N)c1Cl]

The title compound was obtained using a procedure analogous to that of example 2.1, starting from compound 6.2.

Purification of the crude compound by column chromatography (silica gel, 35% EtOAc in hexane) provided the title compound as cream colored solid.

Wt of the product: 0.002 g (18%)

¹H NMR (400 MHz, CDCl₃): δ 7.63 (d, J=7.8 Hz; 1H), 7.22 (d, J=8.4 Hz; 1H), 3.04 (s, 3H), 2.82-2.72 (m, 1H), 2.54-2.39 (m, 3H), 2.38-2.28 (m, 1H), 2.27 (s, 3H), 2.25-2.24 (m, 1H);

IR (KBr): 2957, 2239, 1768, 1719, 1591, 1479, 1406, 1348, 1143 cm⁻¹;

MS (ES): m/z 354.3 (M+1);

MP: 184° C.

### Example 7.0

### Preparation of 2-chloro-4-(4-imino-1-methyl-2-oxo-1,3-diazaspiro[4.4]nonan-3-yl)-3-methylbenzonitrile 7.0

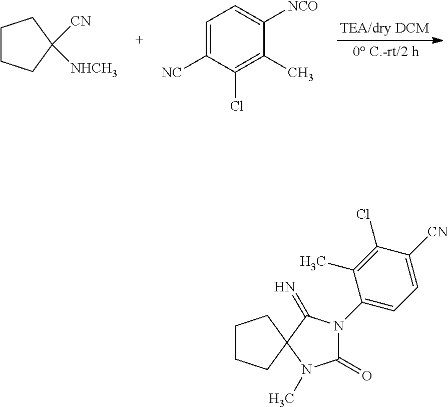

Triethylamine (2.51 mL, 0.02 moles) was added drop wise to a stirred mixture of 1-(methylamino)cyclopentanecarbonitrile (building block B3) (1.50 g, 0.01 moles) in dichloromethane (15 mL) and 2-chloro-4-isocyanato-3-methylbenzonitrile (building block A2) (2.33 g, 0.01 moles) at 0° C. The reaction mixture was stirred at room temperature for 2 h. Once the starting material was consumed (monitored by TLC), the reaction mixture was concentrated under reduced pressure. The residue was extracted with dichloromethane (3×150 mL). The organic layer was washed with brine, dried over Na₂SO₄ and concentrated under reduced pressure. Purification by column chromatography (silica gel, 35% EtOAc in hexane) provided the title compound as a white solid (0.45 g, 12%)

¹H NMR (400 MHz, CDCl₃): δ 7.64 (d, J=7.8 Hz, 1H), 7.25 (d, J=7.8 Hz, 1H), 7.10-6.50 (m, 1H), 2.97 (s, 3H), 2.26 (s, 3H), 2.11 (br s, 4H), 1.94 (br s, 4H);

IR (KBr): 3242, 2959, 2235, 1732, 1663, 1591 cm⁻¹;

MS (ES): m/z 317.2 (M+1);

MP: 199° C.

### Example 8.0

### Preparation of 2-chloro-4-(1-(4-cyanobenzyl)-2,4-dioxo-1,3-diazaspiro[4.4]nonan-3-yl)-3-methylbenzonitrile 8.0

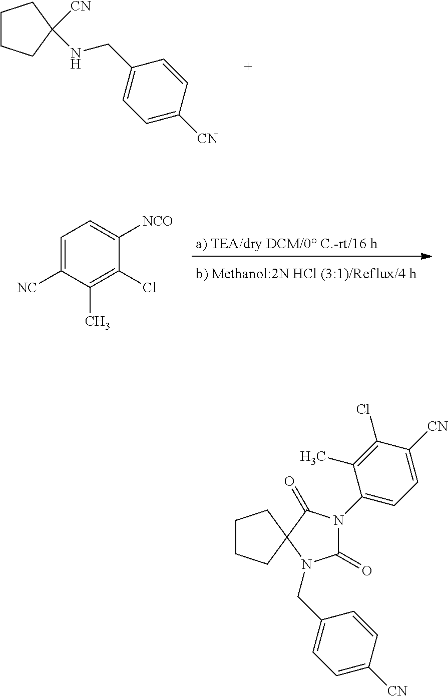

Triethylamine (0.71 mL, 0.005 moles) was added drop wise to a stirred mixture of 4-(((1-cyanocyclopentyl)amino)methyl)benzonitrile (building block B8) (0.77 g, 0.003 moles) in dichloromethane (10 mL) and 2-chloro-4-isocyanato-3-methylbenzonitrile (building block A2) (0.66 g, 0.003 moles) at 0° C. The reaction mixture was stirred at room temperature for 16 h. Once the starting material was consumed (monitored by TLC), the reaction mixture was concentrated under reduced pressure. The residue was dissolved in methanol (10 mL) and 2N HCl (4 mL) and heated to reflux for 4 h. The reaction mixture was allowed to cool to room temperature, poured on crushed ice and extracted with ethyl acetate (3×50 mL). The organic layer was washed with brine solution, dried over Na₂SO₄ and concentrated under reduced pressure. Purification by preparative HPLC (Phenomenex Luna C-18 (2) (250×21.2 mm); 10.5 μm; Mobile phase A: 0.1% TFA; B: ACN; Wavelength: 200-400 nm.), solubility: MeOH+DMSO+water+ACN) provided the title compound as a cream solid (0.030 g, 5%).

¹H NMR (400 MHz, CDCl₃): δ 7.70-7.65 (dd, J, =8.4 Hz & J₂=2.4 Hz; 3H), 7.46 (d, J=8.3 Hz, 2H), 7.29 (d, J=7.3 Hz, 1H), 4.65 (s, 2H), 2.30 (s, 3H), 2.21-2.14 (m, 2H), 1.95-1.80 (m, 6H);

IR (KBr): 2918, 2231, 1769, 1715, 1687, 1556 cm⁻¹;

MS (ES): m/z 417.1 (M−1);

MP: 195° C.

### Example 8.1

### Preparation of 2-chloro-3-methyl-4-(1-((5-methylisoxazol-3-yl)methyl)-2,4-dioxo-1,3-diazaspiro[4.4]nonan-3-yl)benzonitrile (8.1)

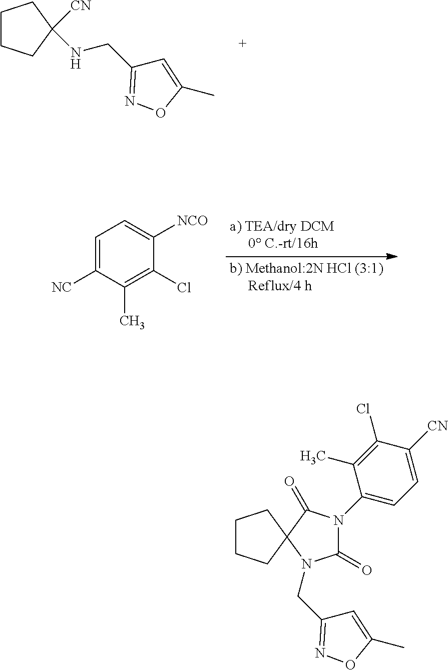

Triethylamine (0.22 mL, 2 mmol) was added drop wise to a stirred mixture of 1-(((5-methylisoxazol-3-yl)methyl)amino)cyclopentanecarbonitrile (building block B9) (0.22 g, 1 mmol) in dichloromethane (5 mL) and 2-chloro-4-isocyanato-3-methylbenzonitrile (building block A2) (0.73 g, 3.8 mmol) at 0° C. The reaction mixture was stirred at room temperature for 16 h. Once the starting material was consumed (monitored by TLC), the reaction mixture was concentrated under reduced pressure. The residue was dissolved in methanol (10 mL) and 2N HCl (4 mL) and heated to reflux for 4 h. The reaction mixture was allowed to cool to room temperature, poured on crushed ice and extracted with ethyl acetate (3×100 mL). The organic layer was washed with brine solution, dried over Na₂SO₄ and concentrated under reduced pressure. Purification by column chromatography (silica gel, 25% EtOAc in hexane) provided the title compound as a white solid (15 mg, 5%).

¹H NMR (400 MHz, DMSO): δ 8.00 (d, J=8.3 Hz; 1H), 7.62 (d, J=8.3 Hz, 1H), 6.27 (s, 1H), 4.60 (s, 2H), 2.40 (s, 3H), 2.21 (s, 3H), 2.08-2.02 (m, 3H), 1.79 (br s, 5H);

IR (KBr): 2957, 2237, 1773, 1715, 1603 cm⁻¹;

MS (LC-MS): m/z 399.1 (M+1);

MP: 134° C.

### Example 8.2

### Preparation of 2-chloro-4-(2,4-dioxo-1-(2-(pyridin-4-yl)ethyl)-1,3-diazaspiro[4.4]nonan-3-yl)-3-methylbenzonitrile 8.2

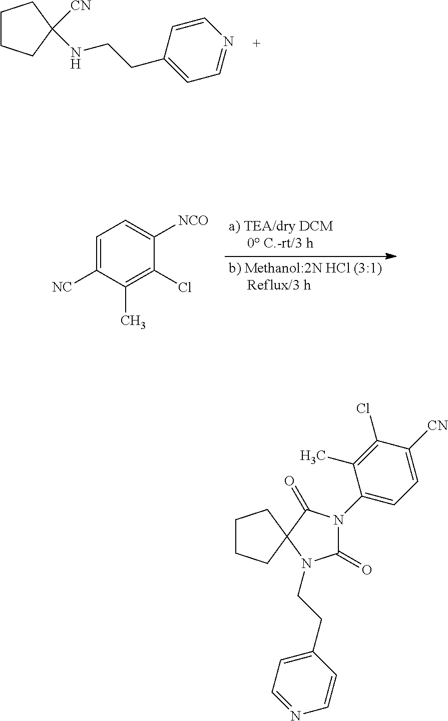

The title compound was synthesized using similar procedure which was used for the synthesis of example 8.1 using 1-((2-(pyridin-4-yl)ethyl)amino)cyclopentanecarbonitrile (building block B10) and 2-chloro-4-isocyanato-3-methylbenzonitrile (building block A2) as the starting materials. Purification by column chromatography (silica gel, 25% EtOAc in hexane) provided the title compound as colorless gummy solid (0.003 g, 1%).

¹H NMR (400 MHz, CDCl₃): δ 8.57 (d, J=5.4 Hz; 2H), 7.63 (d, J=8.3 Hz, 1H), 7.24 (d, J=8.3 Hz, 1H), 7.20 (d, J=5.4 Hz, 2H), 3.65-3.52 (m, 2H), 3.16-3.05 (m, 2H), 2.25 (s, 3H), 2.18-2.09 (m, 2H), 2.07-1.93 (m, 2H), 1.90-1.74 (m, 4H);

MS (LC-MS): m/z 409.1 (M+1).

### Example 8.3

### Preparation of 2-chloro-4-(1-((3,5-dimethylisoxazol-4-yl)methyl)-2,4-dioxo-1,3-diazaspiro[4.4]nonan-3-yl)-3-methylbenzonitrile 8.3

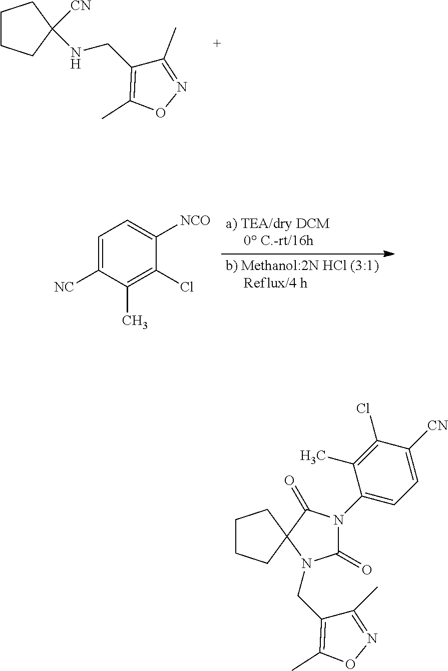

The title compound was synthesized using an analogous procedure used for example 8.1 using 1-(((3,5-dimethylisoxazol-4-yl)methyl)amino)cyclopentanecarbonitrile (building block B16) and 2-chloro-4-isocyanato-3-methylbenzonitrile (building block A2) as starting materials. Purification by column chromatography (silica gel, 40% EtOAc in hexane) provided the title compound as white solid (0.005 g, 2%).

¹H NMR (400 MHz, DMSO): δ 8.00 (d, J=8.3 Hz; 1H), 7.60 (d, J=8.4 Hz, 1H), 4.49-4.40 (m, 2H), 2.41 (S, 3H), 2.21 (s, 6H), 2.16-1.95 (m, 4H), 1.98-1.74 (m, 4H);

IR (KBr): 2962, 2235, 1774, 1714 cm⁻¹;

MS (LC-MS): m/z 413.2 (M+1);

MP: 109° C.

### Example 8.4

### Preparation of 2-chloro-4-(2,4-dioxo-1-(pyridin-2-ylmethyl)-1,3-diazaspiro[4.4]nonan-3-yl)-3-methylbenzonitrile 8.4

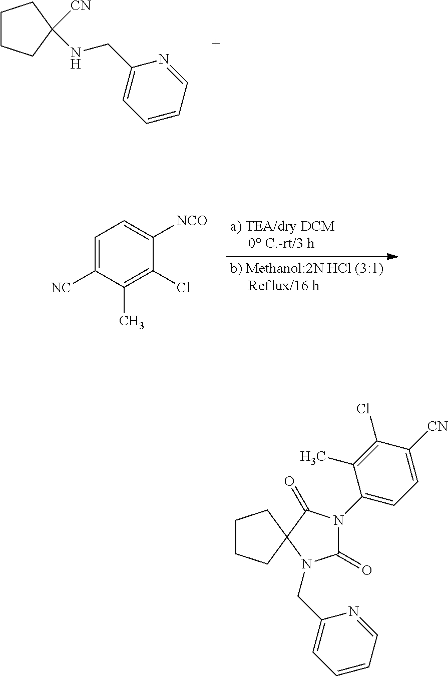

The title compound was synthesized using an analogous procedure used for the synthesis of example 8.1 using 1-((pyridin-2-ylmethyl)amino)cyclopentanecarbonitrile (building block B17) and 2-chloro-4-isocyanato-3-methylbenzonitrile (building block A2) as the starting material. Purification by column chromatography (silica gel, 60% EtOAc in hexane) provided the title compound as white solid (0.08 g, 20%)

¹H NMR (400 MHz, CDCl₃): δ 8.55 (d, J=4.2 Hz; 1H), 7.71-7.61 (m, 3H), 7.39 (d, J=7.9 Hz, 1H), 7.29 (d, J=8.4 Hz, 1H), 7.25-7.22 (m, 1H), 4.71 (d, J=3.7 Hz; 2H), 2.30 (s, 3H), 2.17-2.01 (m, 2H), 2.00-1.84 (m, 6H);

IR (KBr): 3084, 2964, 2929, 2862, 2237, 1768, 1712, 1593 cm⁻¹;

MS (LC-MS): m/z 395.1 (M+1);

MP: 179° C.

### Example 8.5

### Preparation of 2-chloro-4-(2,4-dioxo-1-(pyridin-4-ylmethyl)-1,3-diazaspiro[4.4]nonan-3-yl)-3-methylbenzonitrile 8.5

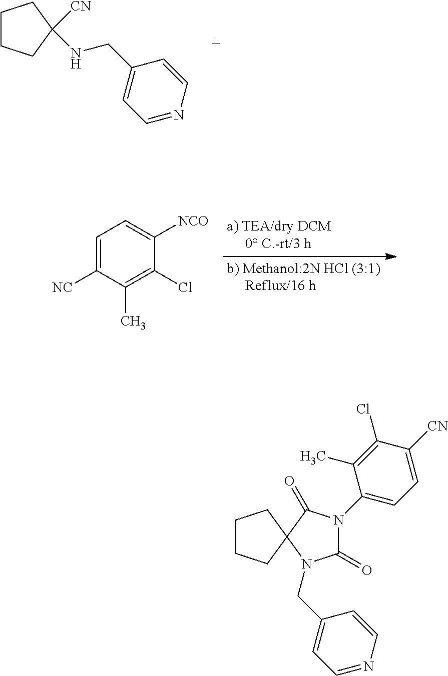

The title compound was synthesized using an analogous procedure used for example 8.1 using 1-((pyridin-4-ylmethyl)amino)cyclopentanecarbonitrile (building block B18) and 2-chloro-4-isocyanato-3-methylbenzonitrile (building block A2) as the starting material. Purification by column chromatography (silica gel, 70% EtOAc in hexane) provided the title compound as white solid (0.16 g, 41%).

¹H NMR (400 MHz, CDCl₃): δ 8.62 (d, J=4.4 Hz; 1H), 7.65 (d, J=8.3 Hz; 1H), 7.29 (d, J=8.3 Hz, 1H), 7.25 (d, J=5.9 Hz, 2H), 4.59 (d, J=2.9 Hz; 2H), 2.31 (s, 3H), 2.20-2.14 (m, 2H), 1.95-1.80 (m, 6H);

IR (KBr): 2962, 2872, 2235, 1774, 1720, 1600, 1562 cm⁻¹;

MS (LC-MS): m/z 395.1 (M+1);

MP: 76° C.

### Example 8.6

### Preparation of 2-chloro-3-methyl-4-(1-((6-methylpyridin-3-yl)methyl)-2,4-dioxo-1,3-diazaspiro[4.4]nonan-3-yl)benzonitrile 8.6

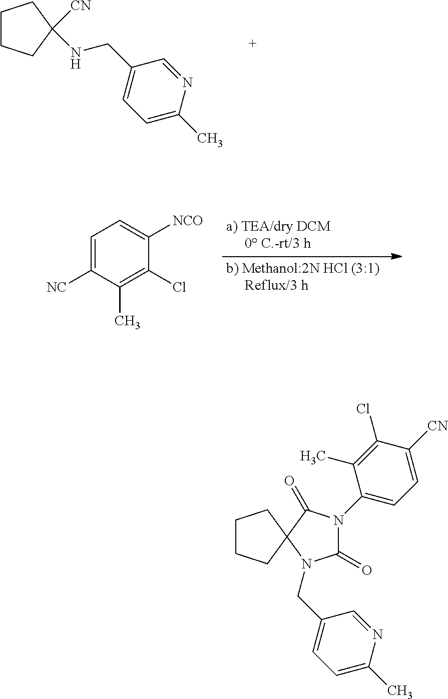

The title compound was synthesized using a analogous procedure used for example 8.1 using 1-(((6-methylpyridin-3-yl)methyl)amino)cyclopentanecarbonitrile (building block B21) and 2-chloro-4-isocyanato-3-methylbenzonitrile (building block A2) as the starting materials. Purification by column chromatography (silica gel, 60% EtOAc in hexane) provided the title compound as a white solid (0.02 g, 6%).

¹H NMR (400 MHz, DMSO): δ 8.48 (s, 1H), 8.01 (d, J=8.3 Hz, 1H), 7.70-7.66 (m, 2H), 7.24 (d, J=8.3 Hz, 1H), 4.65-4.56 (m, 2H), 2.45 (s, 3H), 2.23 (s, 3H), 2.09-1.86 (m, 4H), 1.76-1.74 (m, 4H);

IR (KBr): 2961, 2236, 1773, 1719 cm⁻¹;

MS (LC-MS): m/z 409.2 (M+1);

MP: 177° C.

### Example 8.7

### Preparation of 2-chloro-3-methyl-4-(1-((5-methyloxazol-2-yl)methyl)-2,4-dioxo-1,3-diazaspiro[4.4]nonan-3-yl)benzonitrile 8.7

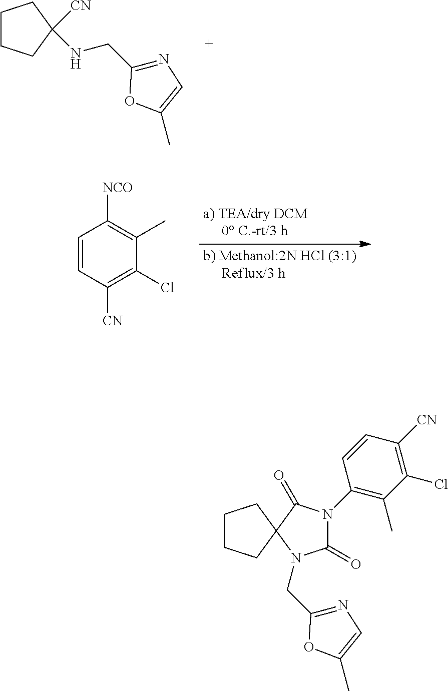

The title compound was synthesized using an analogous procedure used for example 8.1 using 1-(((5-methyloxazol-2-yl)methyl)amino)cyclopentanecarbonitrile (building block B27) and 2-chloro-4-isocyanato-3-methylbenzonitrile (building block A2) as the starting materials. Purification by column chromatography (silica gel, 30% EtOAc in hexane) provided the title compound (4 mg, 2%) as a pale yellow gummy solid.

¹H NMR (400 MHz, CDCl₃): δ7.62 (d, J=7.8 Hz, 1H), 7.29 (d, J=8.3 Hz, 1H), 6.70 (s, 1H), 4.67 (s, 2H), 2.32 (s, 3H), 2.28 (s, 3H), 2.20-2.14 (m, 2H), 2.0-1.86 (m, 6H);

IR (Neat): 3018, 2918, 1720, 1411 cm⁻¹;

MS (ES): m/z 399 (M+1).

### Example 8.8

### 2-chloro-4-(1-((5-(hydroxymethyl)oxazol-2-yl)methyl)-2,4-dioxo-1,3-diazaspiro[4.4]nonan-3-yl)-3-methylbenzonitrile 8.8

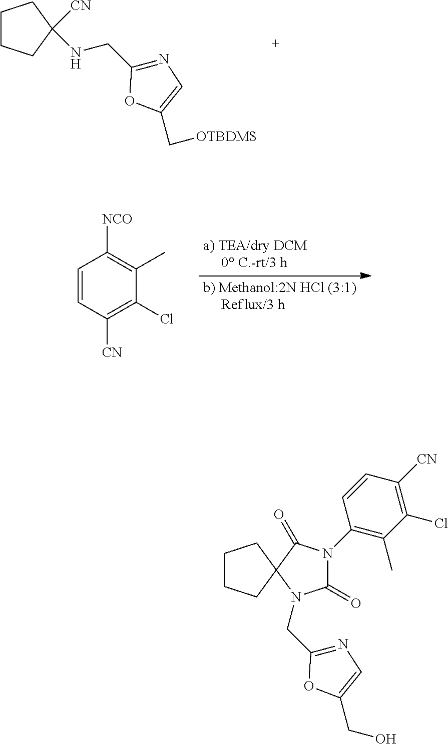

The title compound was synthesized using an analogous procedure to that used for example 8.1 using 1-(((5-(((tert-butyldimethylsilyl)oxy)methyl)oxazol-2-yl)methyl)amino)cyclopentanecarbonitrile (building block B28) and 2-chloro-4-isocyanato-3-methylbenzonitrile (building block A2) as the starting materials. Purification by column chromatography (silica gel, 4% MeOH in DCM) provided the title compound (22 mg, 14%) as a pale yellow color solid.

¹H NMR (400 MHz, DMSO-d₆): δ 7.99 (d, J=8.3 Hz, 1H), 7.60 (s, 1H), 7.59 (d, J=8.3 Hz, 1H), 5.17 (t, J=5.8 Hz, 1H), 4.72 (s, 2H), 4.34 (d, J=4.9 Hz, 2H), 2.20 (s, 3H), 2.03-1.98 (m, 4H), 1.82-1.75 (m, 4H);

IR (KBr): 2958, 2872, 2237, 1776, 1720, 1479 cm⁻¹;

LCMS: m/z 415 (M+1).

### Example 8.9

### Preparation of 2-chloro-3-methyl-4-(1-(oxazol-5-ylmethyl)-2,4-dioxo-1,3-diazaspiro[4.4]nonan-3-yl)benzonitrile 8.9

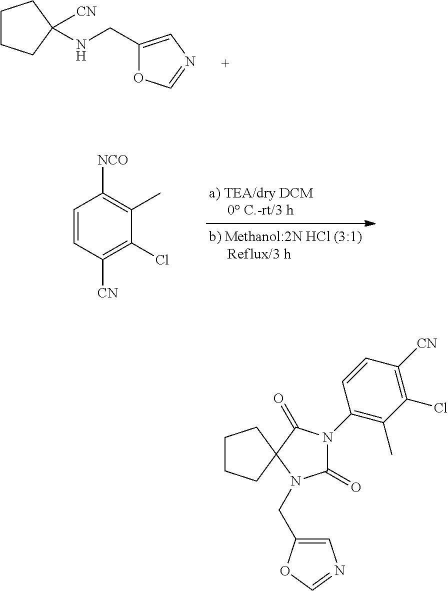

The title compound was synthesized using an analogous procedure to that used for example 8.1 using 1-((oxazol-5-ylmethyl)amino)cyclopentanecarbonitrile (building block B29) and 2-chloro-4-isocyanato-3-methylbenzonitrile (building block A2) as the starting material. Purification by column chromatography (silica gel, 30% EtOAc in hexane) provided the title compound (20 mg, 5%) as a pale yellow color gummy solid.

¹H NMR (400 MHz, DMSO-d₆): δ 8.36 (s, 1H), 7.99 (d, J=8.3 Hz, 1H), 7.61 (d, J=8.3 Hz, 1H), 7.20 (s, 1H), 4.70 (s, 2H), 2.20 (s, 3H), 2.11-1.97 (m, 4H), 1.80-1.78 (m, 4H);

IR (Neat): 3128, 2960, 2873, 2235, 1776, 1716, 1413 cm⁻¹;

LCMS: m/z 385 (M+1).

### Example 8.10

### 2-chloro-3-methyl-4-(1-((2-methyloxazol-5-yl)methyl)-2,4-dioxo-1,3-diazaspiro[4.4]nonan-3-yl)benzonitrile (8.10)

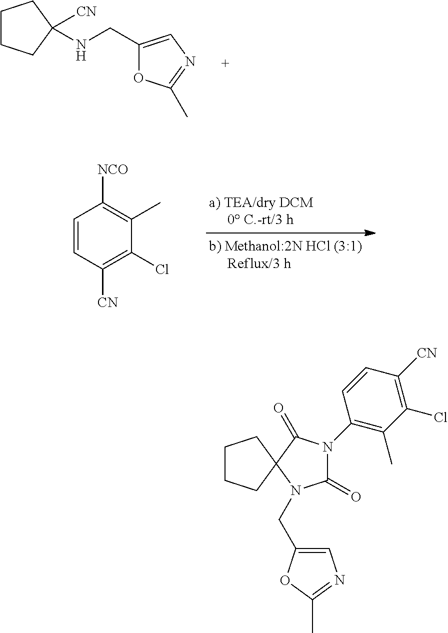

The title compound was synthesized using an analogous procedure to that used for example 8.1 using 1-(((2-methyloxazol-5-yl)methyl)amino)cyclopentanecarbonitrile (building block B30) and 2-chloro-4-isocyanato-3-methylbenzonitrile (building block A2) as the starting material. Purification by column chromatography (silica gel, 30% EtOAc in hexane) provided the title compound (5 mg, 2%) as an off-white solid.

¹H NMR (400 MHz, DMSO-d₆): δ7.99 (d, J=8.3 Hz, 1H), 7.94 (s, 1H), 7.60 (d, J=8.3 Hz, 1H), 4.41 (d, J=3.4 Hz, 2H), 2.39 (s, 3H), 2.20 (s, 3H), 2.08-2.03 (m, 4H), 1.81-1.77 (m, 4H);

LCMS: m/z 399 (M+1).

### Example 9.0

### 2-chloro-4-(1-(2-fluoroethyl)-2,4-dioxo-1,3-diazaspiro[4.4]nonan-3-yl)-3-methylbenzonitrile 9.0

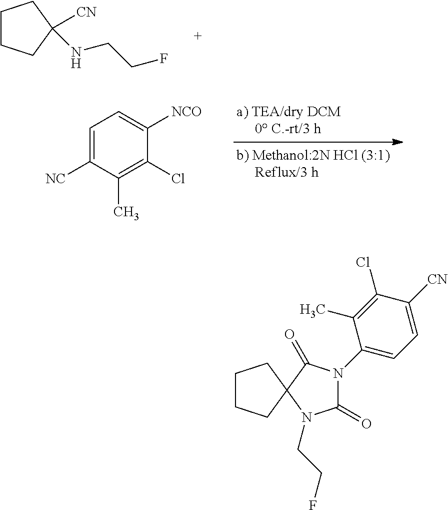

The title compound was synthesized using a similar procedure which was used for the synthesis of example 8.1 using 1-((2-fluoroethyl)amino)cyclopentanecarbonitrile (building block B11) and 2-chloro-4-isocyanato-3-methylbenzonitrile (building block A2) as the starting material. Purification by column chromatography (silica gel, 20% EtOAc in hexane) provided the title compound as cream coloured solid (6 mg, 2%).

¹H NMR (400 MHz, CDCl₃): δ 7.63 (d, J=8.3 Hz; 1H), 7.26 (d, J=8.3 Hz, 1H), 4.78-4.76 (t, J=4.4 Hz, 1H), 4.66-4.64 (t, J=4.4 Hz, 1H), 3.68-3.66 (t, J=3.9 Hz, 1H), 3.61-3.59 (t, J=3.9 Hz, 1H), 2.27 (s, 3H), 2.23-2.20 (m, 2H), 2.07-1.92 (m, 6H);

IR (KBr): 2965, 2232, 1769, 1713, 1591 cm⁻¹;

MP: 123° C.

### Example 9.1

### Preparation of 2-chloro-4-(1-(5-cyanopentyl)-2,4-dioxo-1,3-diazaspiro[4.4]nonan-3-yl)-3-methylbenzonitrile 9.1

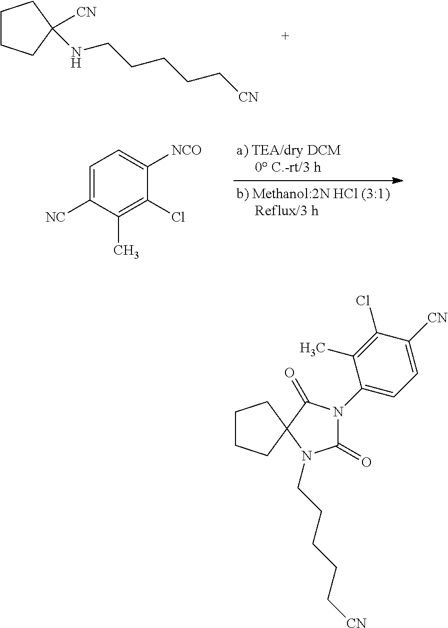

The title compound was synthesized using a similar procedure used for the synthesis of example 8.1 using 1-((5-cyanopentyl)amino)cyclopentanecarbonitrile (building block B12) and 2-chloro-4-isocyanato-3-methylbenzonitrile (building block A2) as the starting material. Purification by column chromatography (silica gel, 30% EtOAc in hexane) provided the title compound as white gummy solid (0.045 g, 2%).

¹H NMR (400 MHz, CDCl₃): δ 7.98 (d, J=8.4 Hz; 1H), 7.57 (d, J=8.3 Hz, 1H), 3.37-3.27 (m, 4H), 2.19 (s, 3H), 2.12-2.09 (m, 1H), 2.03 (br s, 3H), 1.85-1.82 (m, 4H), 1.71-1.56 (m, 4H), 1.45-1.38 (m, 2H);

IR (KBr): 2936, 2235, 1773, 1717, 1591 cm⁻¹;

MS (LC-MS): m/z 399.2 (M+1).

### Example 9.2

### 2-chloro-4-(1-(2-(2-fluoroethoxy)ethyl)-2,4-dioxo-1,3-diazaspiro[4.4]nonan-3-yl)-3-methylbenzonitrile 9.2

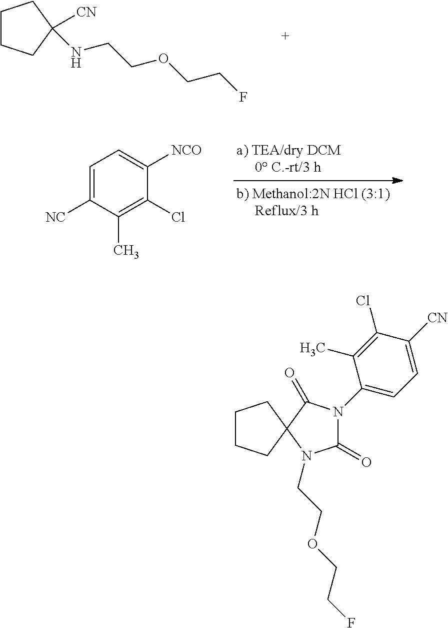

The title compound was synthesized using similar procedure used for the synthesis of example 8.1 using 1-((2-(2-fluoroethoxyl)ethyl)amino)cyclopentanecarbonitrile (building block B13) and 2-chloro-4-isocyanato-3-methylbenzonitrile (building block A2) as the starting material. Purification by column chromatography (silica gel, 12% EtOAc in hexane) provided the title compound as white solid (0.060 g, 12%).

¹H NMR (400 MHz, DMSO): δ 7.98 (d, J=8.3 Hz; 1H), 7.56 (d, J=8.3 Hz, 1H), 4.59-4.45 (m, 2H), 3.73-3.63 (m, 4H), 3.52-3.44 (m, 2H), 2.24-2.01 (m, 6H), 1.98-1.77 (m, 4H);

MS (LC-MS): m/z 394.1 (M+1);

MP: 112° C.

### Example 9.3

### Preparation of 2-chloro-4-(1-(2-(cyanomethoxy)ethyl)-2,4-dioxo-1,3-diazaspiro[4.4]nonan-3-yl)-3-methylbenzonitrile 9.3

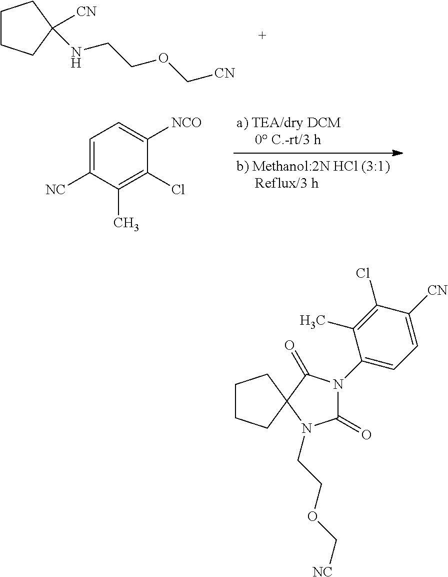

The title compound was synthesized using similar procedure used for the synthesis of example 8.1 using 1-((2-(cyanomethoxy)ethyl)amino)cyclopentanecarbonitrile (building block B14) and 2-chloro-4-isocyanato-3-methylbenzonitrile (building block A2) as the starting materials. Purification by column chromatography (silica gel, 10% EtOAc in hexane) provided the title compound as a thick liquid (0.020 g, 5%).

¹H NMR (400 MHz, DMSO): δ 7.99 (d, J=7.8 Hz; 1H), 7.56 (d, J=7.8 Hz, 1H), 4.53 (s, 2H), 3.76 (d, J=4.9 Hz; 1H), 3.52 (s, 2H), 2.32-2.07 (m, 7H), 1.82-1.26 (m, 4H);

MS (LC-MS): m/z 387.1 (M+1).

### Example 9.4

### Preparation of 2-chloro-4-(1-(3-cyanopropyl)-2,4-dioxo-1,3-diazaspiro[4.4]nonan-3-yl)-3-methylbenzonitrile 9.4

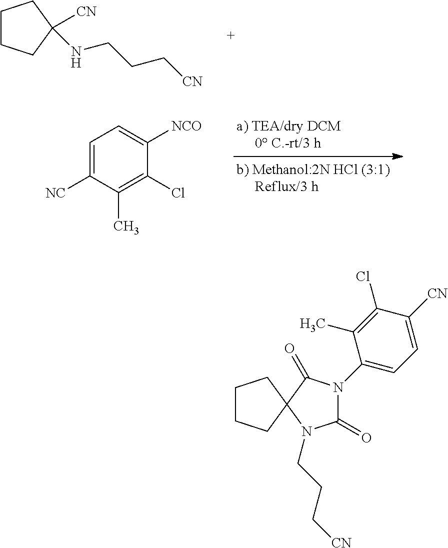

The title compound was synthesized using a analogous procedure used for the synthesis of example 8.1 using 1-((3-cyanopropyl)amino)cyclopentanecarbonitrile (building block B19) and 2-chloro-4-isocyanato-3-methylbenzonitrile (building block A2) as the starting materials. Purification by column chromatography (silica gel, 50% EtOAc in hexane) provided the title compound as white gummy solid (8 mg, 4%).

¹H NMR (400 MHz, CDCl₃): δ 7.62 (d, J=8.3 Hz; 1H), 7.26 (d, J=6.4 Hz, 1H), 3.55-3.42 (m, 2H), 2.53-2.49 (m, 2H), 2.26 (s, 3H), 2.23 (t, J=7.1 Hz; 2H), 2.15-2.10 (m, 2H), 2.00-1.90 (m, 6H);

IR (KBr): 2958, 2872, 2235, 1772, 1716, 1591 cm⁻¹;

MS (LC-MS): m/z 371.0 (M+1).

### Example 9.5

### Preparation of 2-chloro-4-(1-isobutyl-2,4-dioxo-1,3-diazaspiro[4.4]nonan-3-yl)-3-methylbenzonitrile 9.5

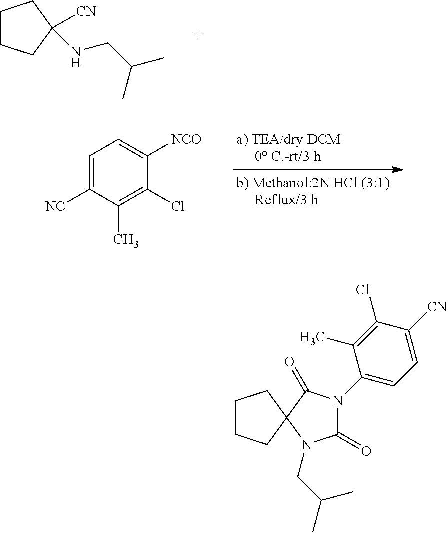

The title compound was synthesized using an analogous procedure to example 8.1 using 1-(isobutylamino)cyclopentanecarbonitrile (building block B20) and 2-chloro-4-isocyanato-3-methylbenzonitrile (building block A2) as the starting materials. Purification by column chromatography (silica gel, 15% EtOAc in hexane) provided the title compound as white solid (0.14 g, 14%).

¹H NMR (400 MHz, CDCl₃): δ 7.61 (d, J=8.3 Hz; 1H), 7.24 (d, J=8.3 Hz, 1H), 3.21-3.09 (m, 2H), 2.25 (s, 3H), 2.22-2.11 (m, 3H), 2.04-1.91 (m, 4H), 1.89-1.86 (m, 2H), 0.98 (d, J=6.8 Hz; 6H);

IR (KBr): 2960, 2872, 2235, 1772, 1716, 1591 cm⁻¹;

MS (LC-MS): m/z 360.2 (M+1);

MP: 115° C.

### Example 9.6

### Preparation of 2-chloro-4-(1-(4-cyanobutyl)-2,4-dioxo-1,3-diazaspiro[4.4]nonan-3-yl)-3-methylbenzonitrile 9.6

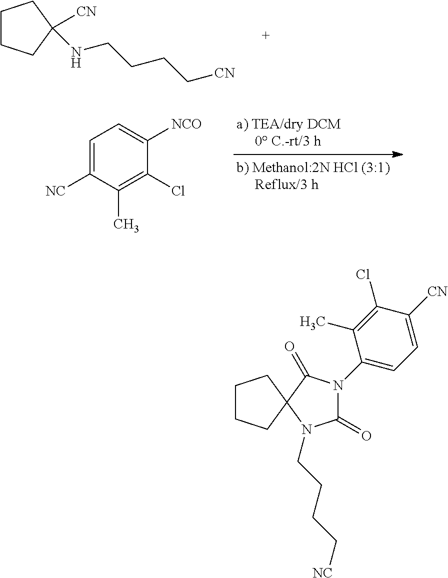

The title compound was synthesized using a analogous procedure used for example 8.1 using 1-((4-cyanobutyl)amino)cyclopentanecarbonitrile (building block B22) and 2-chloro-4-isocyanato-3-methylbenzonitrile (building block A2) as the starting material. Purification by column chromatography (silica gel, 40% EtOAc in hexane) provided the title compound as a colorless semi solid (1.0 g, 17%).

¹H NMR (400 MHz, DMSO): δ 7.98 (d, J=8.3 Hz; 1H), 7.58 (d, J=8.3 Hz, 1H), 2.58-2.54 (m, 3H), 2.22-2.19 (m, 3H), 2.13-2.12 (m, 1H), 2.10-1.98 (m, 3H), 1.86-1.70 (m, 6H), 1.66-1.59 (m, 2H);

IR (KBr): 3445, 2959, 2872, 2236, 1773, 1717 cm⁻¹;

MS (LC-MS): m/z 385.2 (M+1).

### Example 9.7

### Preparation of 2-chloro-4-(1-(2-hydroxyethyl)-2,4-dioxo-1,3-diazaspiro[4.4]nonan-3-yl)-3-methylbenzonitrile 9.7

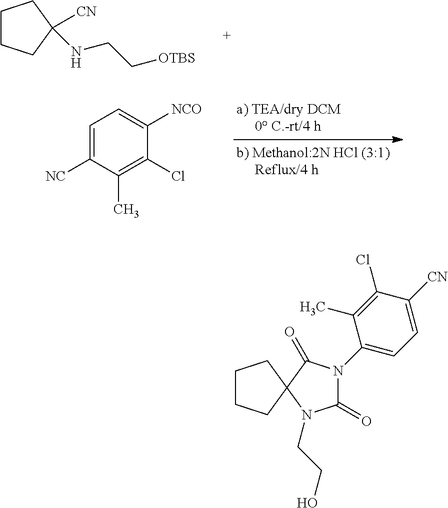

The title compound was synthesized using an analogous procedure to example 8.1 using 1-((2-((tert-butyldimethylsilyl)oxy)ethyl)amino)cyclopentanecarbonitrile (building block B23) and 2-chloro-4-isocyanato-3-methylbenzonitrile (building block A2) as starting materials. Purification by column chromatography (silica gel, 60% EtOAc in hexane) provided the title compound as a white solid (0.52 g, 22%).

¹H NMR (400 MHz, DMSO): δ 7.98 (d, J=8.4 Hz; 1H), 7.55 (d, J=8.3 Hz, 1H), 4.91 (t, J=5.9 Hz; 1H), 3.65-3.60 (m, 2H), 3.35-3.31 (m, 2H), 2.20 (s, 3H), 2.14-2.00 (m, 4H), 1.85-1.76 (m, 4H);

IR (KBr): 3532, 2961, 2874, 2236, 1769, 1712 cm⁻¹;

MS (LC-MS): m/z 348.1 (M+1);

MP: 98° C.

### Example 9.8

### Preparation of 2-chloro-4-(1-(2-cyanoethyl)-2,4-dioxo-1,3-diazaspiro[4.4]nonan-3-yl)-3-methylbenzonitrile 9.8

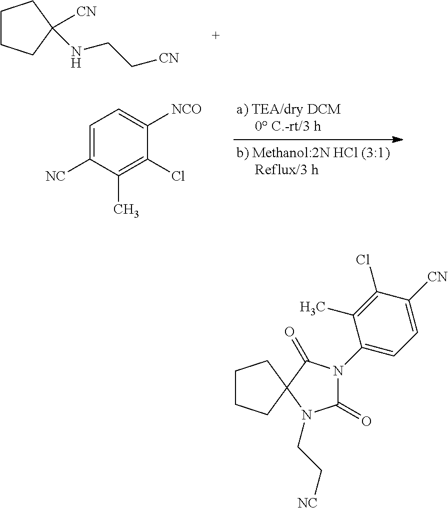

The title compound was synthesized using a analogous procedure used for example 8.1 using 1-((2-cyanoethyl)amino)cyclopentanecarbonitrile (building block B24) and 2-chloro-4-isocyanato-3-methylbenzonitrile (building block A2) as the starting materials. Purification by column chromatography (silica gel, 25% EtOAc in hexane) provided the title compound as a gummy solid (0.040 g, 5%).

¹H NMR (400 MHz, DMSO): δ 7.99 (d, J=8.3 Hz; 1H), 7.58 (d, J=8.4 Hz, 1H), 3.64-3.60 (m, 2H), 2.96-2.88 (m, 2H), 2.14-2.02 (m, 4H), 1.88-1.77 (m, 4H);

IR (KBr): 3431, 2967, 2237, 1775, 1717 cm⁻¹;

MS (LC-MS): m/z 357.1 (M+1).

### Example 9.9

### Preparation of 2-chloro-4-(1-(3-fluoropropyl)-2,4-dioxo-1,3-diazaspiro[4.4]nonan-3-yl)-3-methylbenzonitrile 9.9

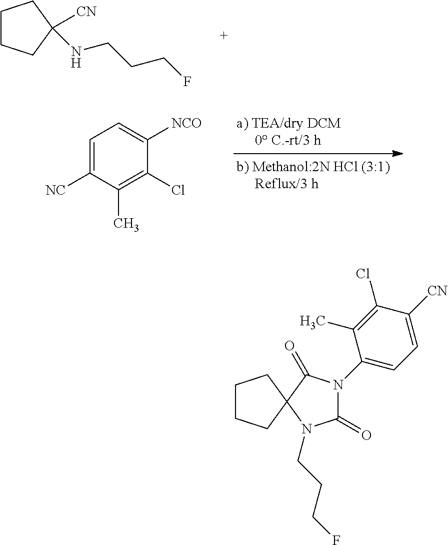

The title compound was synthesized using a analogous procedure used for example 8.1 using 1-((3-fluoropropyl)amino)cyclopentanecarbonitrile (building block B25) and 2-chloro-4-isocyanato-3-methylbenzonitrile (building block A2) as the starting materials. Purification by column chromatography (silica gel, 35% EtOAc in hexane) provided the title compound as a gummy solid (8 mg, 4%).

¹H NMR (400 MHz, DMSO): δ 7.98 (d, J=8.3 Hz; 1H), 7.58 (d, J=8.3 Hz, 1H), 4.62-4.60 (t, J=5.8 Hz, 1H), 4.50-4.48 (t, J=5.4 Hz, 1H), 3.42-3.37 (m, 2H), 2.14-1.90 (m, 6H), 1.83-1.81 (m, 4H);

IR (KBr): 2963, 2930, 2236, 1769, 1713 cm⁻¹;

MS (LC-MS): m/z 364.2 (M+1).

### Example 10.0

### 2-chloro-3-methyl-4-(1-methyl-2,4-dioxo-7-oxa-1,3-diazaspiro[4.4]nonan-3-yl)benzonitrile 10.0

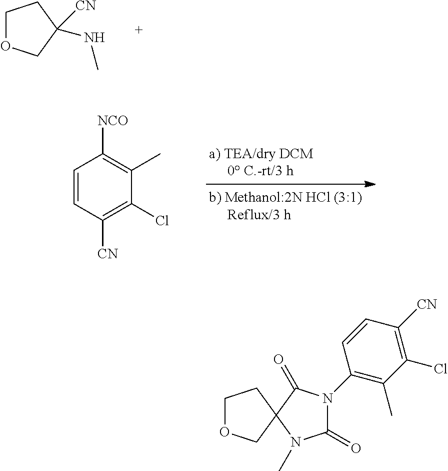

The title compound was synthesized using an analogous procedure to that used for example 8.1 using 3-(methylamino)tetrahydrofuran-3-carbonitrile (building block B31) and 2-chloro-4-isocyanato-3-methylbenzonitrile (building block A2) as the starting material. Purification by column chromatography (silica gel, 30% EtOAc in hexane) provided the title compound (180 mg). Two isomers were separated by preparative HPLC method.

### Preparative HPLC Conditions:

Column Name: Lux cellulose-2 (250 mm×21.1 mm), 5 μm

Mobile phase: A: n-Hexane; B: Isopropanol

Gradient: ISOCRATIC, (A:B) (40:60)

Flow rate: 17 mL/min

Peak 1 eluted at 17.24 minutes and peak 2 eluted at 22.19 minutes.

### Isomer 1:

15 mg (2%)

¹H NMR (400 MHz, CDCl₃): δ 7.63 (d, J=8.3 Hz, 1H), 7.22 (d, J=8.3 Hz, 1H), 4.28-4.22 (m, 1H), 4.17-4.13 (m, 1H), 4.03-3.95 (m, 2H), 3.08 (s, 3H), 2.62-2.54 (m, 1H), 2.36-2.31 (m, 1H), 2.24 (d, J=12.2 Hz, 3H); IR (KBr): 2958, 2858, 2237, 1774, 1718, 1481, 1406, 829 cm⁻¹; LCMS: m/z 320 (M+1).

### Isomer 2:

33 mg (4%)

¹H NMR (400 MHz, CDCl₃): δ7.63 (d, J=8.3 Hz, 1H), 7.22 (d, J=8.3 Hz, 1H), 4.28-4.22 (m, 1H), 4.17-4.13 (m, 1H), 4.03-3.95 (m, 2H), 3.08 (s, 3H), 2.62-2.54 (m, 1H), 2.36-2.29 (m, 1H), 2.24 (d, J=12.2 Hz, 3H).

### Example 11.0

### Preparation of 2-chloro-4-(2,4-dioxo-1-((tetrahydrofuran-3-yl)methyl)-1,3-diazaspiro[4.4]nonan-3-yl)-3-methylbenzonitrile 11.0

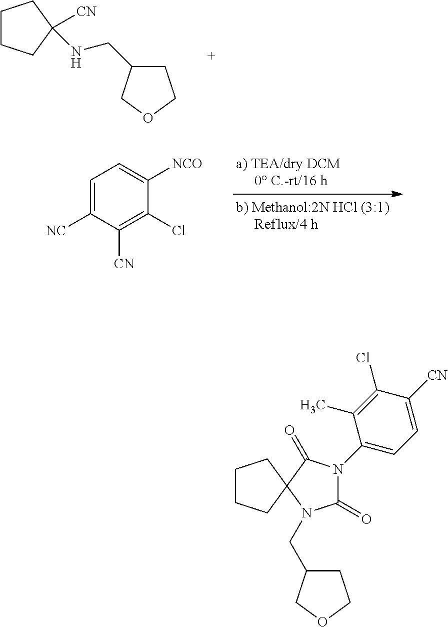

The title compound was synthesized using a similar procedure to that used for example 8.1 using 1-(((tetrahydrofuran-3-yl)methyl)amino)cyclopentanecarbonitrile (building block B32) and 2-chloro-4-isocyanato-3-methylbenzonitrile (building block A2) as starting materials. Purification by column chromatography (silica gel, 40% EtOAc in hexane) provided the title compound as a white solid (60 mg, 30%).

¹H NMR (400 MHz, CDCl₃): δ 7.62 (d, J=8.3 Hz; 1H), 7.24 (d, J=8.3 Hz, 1H), 3.97-3.92 (m, 2H), 3.86-3.77 (m, 2H), 3.62-3.58 (m, 1H), 3.42-3.35 (m, 1H), 3.30-3.23 (m, 1H), 2.81 (t, J=6.8 Hz; 1H), 2.25 (s, 3H), 2.23-2.20 (m, 2H), 2.10-1.90 (m, 5H), 1.73-1.70 (m, 1H);

MS (LC-MS): m/z 388.2 (M+1);

MP: 70° C.

### Example 12.0

### 2-chloro-3-methyl-4-(5-methyl-6,8-5,7-diazaspiro[3.4]octan-7-yl)benzonitrile 12.0

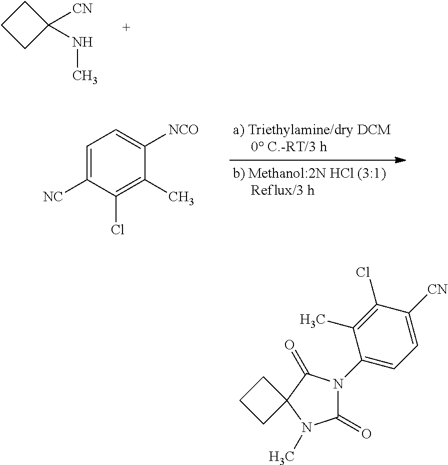

Triethylamine (3.0 mL, 0.02 moles) was added drop wise to a stirred mixture of 1-(methylamino)cyclobutanecarbonitrile (building block B33) (1.57 g, 0.014 moles) in dichloromethane (20 mL) and 2-chloro-4-isocyanato-3-methylbenzonitrile (building block A2) (2.74 g, 0.014 moles) at 0° C. The reaction mixture was stirred at room temperature for 3 h. Once the starting material was consumed (monitored by TLC), the reaction mixture was concentrated under reduced pressure. The residue was dissolved in methanol (15 mL) and 2N HCl (5 mL) and heated to reflux for 3 h. The reaction mixture was allowed to cool to room temperature, poured on crushed ice and extracted with ethyl acetate (3×100 mL). The organic layer was washed with brine solution, dried over Na₂SO₄ and concentrated under reduced pressure. Purification by column chromatography (silica gel, 30% EtOAc in hexane) provided the title compound as a white solid (0.26 g, 7%).

1H NMR (400 MHz, CDCl₃): δ 7.61 (d, J=8.3 Hz; 1H), 7.21 (d, J=8.3 Hz; 1H), 3.12 (s, 3H), 2.63-2.54 (m, 4H), 2.31-2.26 (m, 1H), 2.25 (s, 3H), 1.99-1.92 (m, 1H);

MS (ES): m/z 304.1 (M+1).

MP: 133° C.

### Example 13.0

### Biological Activity of Compounds of Formula (I)

The compounds of the Examples hereinbefore show the following EC₅₀ values in Test 1 described hereinbefore.

### Materials and Methods:

C2C12 cells were obtained from ATCC (Cat #CRL-1772) and maintained in DMEM modified to contain 4 mM L-glutamine, 4.5 g/L glucose, 1 mM sodium pyruvate and 1.5 g/L sodium bicarbonate and 10% FBS.

96-well tissue culture treated plates—clear flat bottom BD Cat #353072
96-well plate white Greiner Cat #655075

### Dihydro Testosterone (DHT) TCI Cat #A0462

### OptiMEM Gibco Cat #31985

### Lipofectamine 2000 Invitrogen Cat #11668-019

AR-FL in pcDNA 3.1(+) and 2XIDR17 in pGL4.26 plasmids prepared using Genelute plasmid miniprep kit from Sigma Cat #PLED35
Steadyglow Luciferase assay system Promega Cat #E2550

### Assay Protocol:

C2C12 cells were seeded in a 96-well plate in DMEM (Dulbecco's Modified Eagle Medium) without phenol red and containing 10% CS-FBS (Charcoal-stripped Fetal Bovine Serum) at 8000 cells/well.
The next day, cells were transfected with an equimolar ratio of (Androgen Receptor-Full length) AR-FL and 2XIDR17-Luciferase at a total plasmid concentration of 200 ng/well using Lipofectamine 2000 following manufacturer's protocol.
For the transfection, 83 ng of AR-FL and 117 ng of 2XIDR17-Luciferase were in 12.5 μl of OptiMEM—Mix A. 0.4 μl of Lipofectamine 2000 was added to 12.5 μl of OptiMEM and incubated for 5 min at room temperature—Mix B. The two mixes A and B were mixed and incubated at room temperature for an additional fifteen minutes. An additional 50 μl of OptiMEM was added, gently mixed and this mixture was added to the cells in the 96-well plate. The above quantities are requirements per well of a 96-well plate. Master mixes were made for the entire plate, with proportional quantity of reagents being used.
5 h after transfection, compounds were added to the wells in DMEM without phenol red and containing 10% CS-FBS, maintaining a final DMSO concentration of 0.5%. A typical dose response curve starts at 10 μM and includes a 7-point, log dilution, done in triplicates.
After overnight incubation with the compounds, 100 ul of working solution of Steadyglow reagent was added to the wells.
The plates were placed in a shaker for 15 min at the end of which the lysate containing luciferase was transferred to a white flat-bottom plate and read under a luminescence setting in Victor.
Background subtracted counts (Luminescence from DMSO control wells is considered the background) are used to calculate percentage activity, expressed relative to activity with 1 μM (Dihydrotestosterone) DHT (at least two sets of triplicates for 1 μM DHT are included per plate).

### Data Fitting:

The EC₅₀ curves for the compounds for 8 compound concentrations were fitted by the respective function using non-linear least-squares regression in Graphpad Prism 4.0 (Graphpad Software, San Diego, Calif., USA).

TABLE 1

| | | Biological activity |
|---|---|---|
| | | (C2C12 cell) |
| Ex. No. | Chemical structure | EC50 (nM) (Emax %) |
| 1.0 | 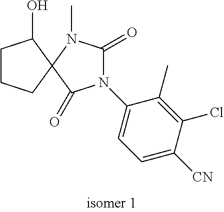 [SMILES: Cc1c(N2C(=O)N(C)C3(CCCC3O)C2=O)ccc(C#N)c1Cl] | 62 (68%) |
| 1.0 | 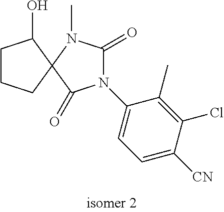 [SMILES: Cc1c(N2C(=O)N(C)C3(CCCC3O)C2=O)ccc(C#N)c1Cl] | 66 (60%) |
| 1.0 | 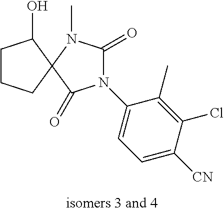 [SMILES: Cc1c(N2C(=O)N(C)C3(CCCC3O)C2=O)ccc(C#N)c1Cl] | 49.9 (80%) |
| 1.1 | 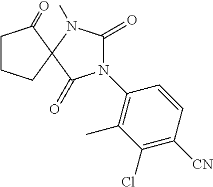 [SMILES: Cc1c(N2C(=O)N(C)C3(CCCC3=O)C2=O)ccc(C#N)c1Cl] | 847 (60%) |
| 1.2 | 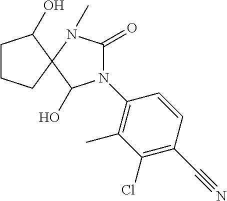 [SMILES: Cc1c(N2C(=O)N(C)C3(CCCC3O)C2O)ccc(C#N)c1Cl] | 930 (79%) |
| 1.3 | 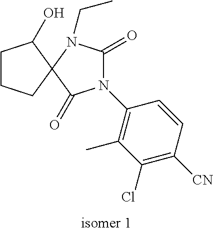 [SMILES: CCN1C(=O)N(c2ccc(C#N)c(Cl)c2C)C(=O)C12CCCC2O] | 173 (79%) |
| 1.3 | 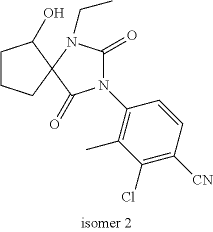 [SMILES: CCN1C(=O)N(c2ccc(C#N)c(Cl)c2C)C(=O)C12CCCC2O] | 139 (72%) |
| 1.4 | 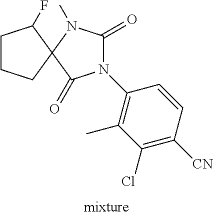 [SMILES: Cc1c(N2C(=O)N(C)C3(CCCC3F)C2=O)ccc(C#N)c1Cl] | 12.6 |
| 1.4 | 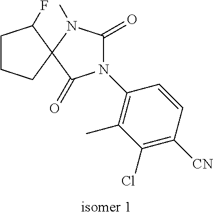 [SMILES: Cc1c(N2C(=O)N(C)C3(CCCC3F)C2=O)ccc(C#N)c1Cl] | 333 (60%) |
| 1.4 | 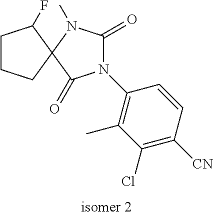 [SMILES: Cc1c(N2C(=O)N(C)C3(CCCC3F)C2=O)ccc(C#N)c1Cl] | 1.43 |
| 1.4 | 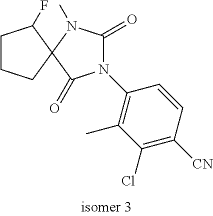 [SMILES: Cc1c(N2C(=O)N(C)C3(CCCC3F)C2=O)ccc(C#N)c1Cl] | 158 |
| 1.4(iv) | 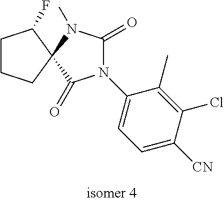 [SMILES: Cc1c(N2C(=O)N(C)[C@]3(CCC[C@@H]3F)C2=O)ccc(C#N)c1Cl] | 0.79 |
| 1.5 | 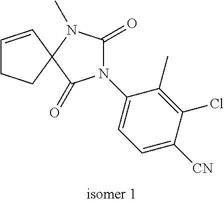 [SMILES: Cc1c(N2C(=O)N(C)C3(C=CCC3)C2=O)ccc(C#N)c1Cl] | 38 (66) |
| 1.5 | 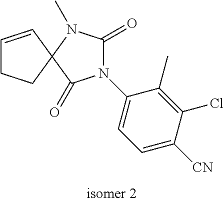 [SMILES: Cc1c(N2C(=O)N(C)C3(C=CCC3)C2=O)ccc(C#N)c1Cl] | 6 |
| 1.6 | 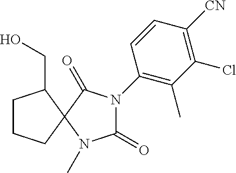 [SMILES: Cc1c(N2C(=O)N(C)C3(CCCC3CO)C2=O)ccc(C#N)c1Cl] | 28/75* |
| 1.7 | 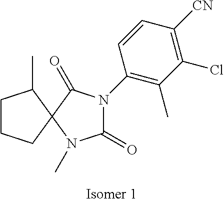 [SMILES: Cc1c(N2C(=O)N(C)C3(CCCC3C)C2=O)ccc(C#N)c1Cl] | 47 + 5 (91 + 5) |
| 1.7 | 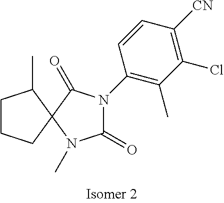 [SMILES: Cc1c(N2C(=O)N(C)C3(CCCC3C)C2=O)ccc(C#N)c1Cl] | 68 + 7 (99 + 4) |
| 1.7 | 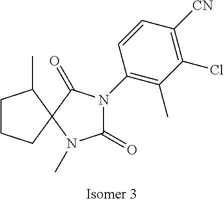 [SMILES: Cc1c(N2C(=O)N(C)C3(CCCC3C)C2=O)ccc(C#N)c1Cl] | 30/63* |
| 1.7 | 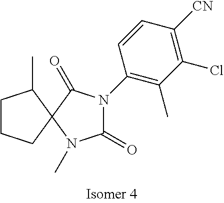 [SMILES: Cc1c(N2C(=O)N(C)C3(CCCC3C)C2=O)ccc(C#N)c1Cl] | 35/52* |
| 2.0 | 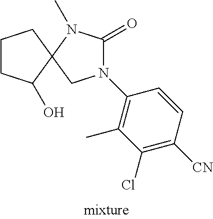 [SMILES: Cc1c(N2CC3(CCCC3O)N(C)C2=O)ccc(C#N)c1Cl] | 7/63* |
| 2.0 | 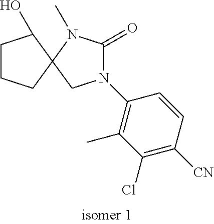 [SMILES: Cc1c(N2CC3(CCCC3O)N(C)C2=O)ccc(C#N)c1Cl] | 2913 (76) |
| 2.0 | 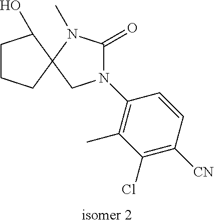 [SMILES: Cc1c(N2CC3(CCCC3O)N(C)C2=O)ccc(C#N)c1Cl] | ** |
| 3.0 | 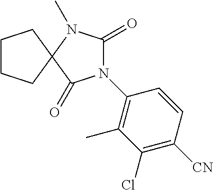 [SMILES: Cc1c(N2C(=O)N(C)C3(CCCC3)C2=O)ccc(C#N)c1Cl] | 0.41 (100) |
| 3.1 | 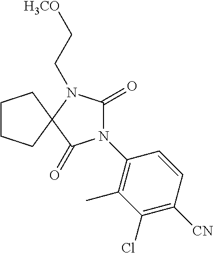 [SMILES: COCCN1C(=O)N(c2ccc(C#N)c(Cl)c2C)C(=O)C12CCCC2] | 105.5 (96) |
| 3.2 | 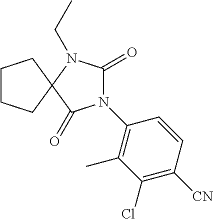 [SMILES: CCN1C(=O)N(c2ccc(C#N)c(Cl)c2C)C(=O)C12CCCC2] | 23.5 (83) |
| 4.0 | 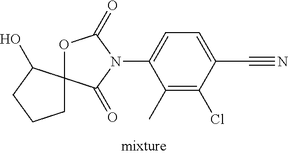 [SMILES: Cc1c(N2C(=O)OC3(CCCC3O)C2=O)ccc(C#N)c1Cl] | 665 (Partial, 34%) |
| 4.0 | 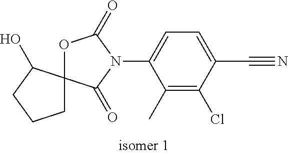 [SMILES: Cc1c(N2C(=O)OC3(CCCC3O)C2=O)ccc(C#N)c1Cl] | 1036 (60%) |
| 4.0 | 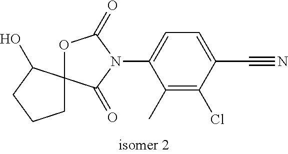 [SMILES: Cc1c(N2C(=O)OC3(CCCC3O)C2=O)ccc(C#N)c1Cl] | 3/18* |
| 4.0 | 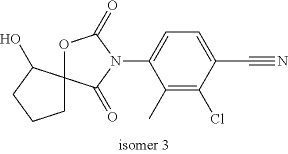 [SMILES: Cc1c(N2C(=O)OC3(CCCC3O)C2=O)ccc(C#N)c1Cl] | 308 (64%) |
| 4.0 | 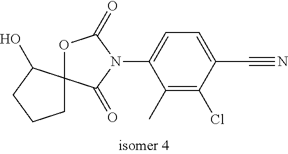 [SMILES: Cc1c(N2C(=O)OC3(CCCC3O)C2=O)ccc(C#N)c1Cl] | 2/17* |
| 4.1 | 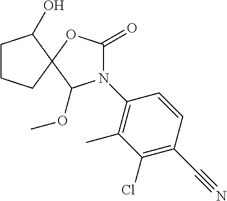 [SMILES: COC1N(c2ccc(C#N)c(Cl)c2C)C(=O)OC12CCCC2O] | 2594 (40%) |
| 5.0 | 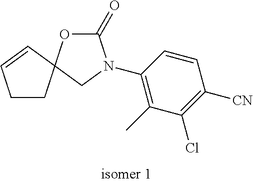 [SMILES: Cc1c(N2CC3(C=CCC3)OC2=O)ccc(C#N)c1Cl] | 2/103* |
| 5.0 | 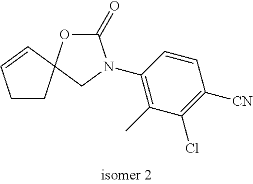 [SMILES: Cc1c(N2CC3(C=CCC3)OC2=O)ccc(C#N)c1Cl] | 2/31* |
| 6.0 | 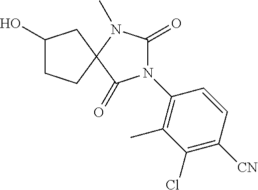 [SMILES: Cc1c(N2C(=O)N(C)C3(CCC(O)C3)C2=O)ccc(C#N)c1Cl] | 53 (70) |
| 6.1 | 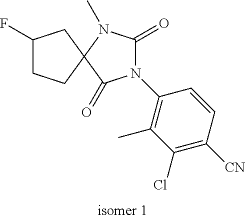 [SMILES: Cc1c(N2C(=O)N(C)C3(CCC(F)C3)C2=O)ccc(C#N)c1Cl] | 34 (99) |
| 6.1 | 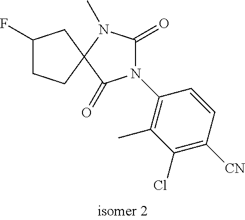 [SMILES: Cc1c(N2C(=O)N(C)C3(CCC(F)C3)C2=O)ccc(C#N)c1Cl] | 11.9 (97) |
| 6.2 | 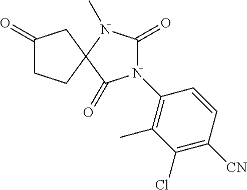 [SMILES: Cc1c(N2C(=O)N(C)C3(CCC(=O)C3)C2=O)ccc(C#N)c1Cl] | 9/29* |
| 6.3 | 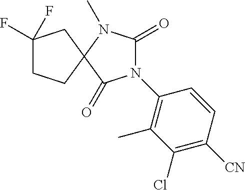 [SMILES: Cc1c(N2C(=O)N(C)C3(CCC(F)(F)C3)C2=O)ccc(C#N)c1Cl] | 14.9 (63) |
| 7.0 | 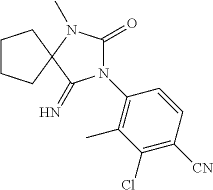 [SMILES: Cc1c(N2C(=N)C3(CCCC3)N(C)C2=O)ccc(C#N)c1Cl] | 103 + 10 (53 + 7) |
| 8.0 | 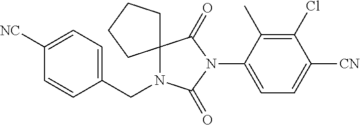 [SMILES: Cc1c(N2C(=O)N(Cc3ccc(C#N)cc3)C3(CCCC3)C2=O)ccc(C#N)c1Cl] | 0.48 + 0.11 (105.5 + 0.7) |
| 8.1 | 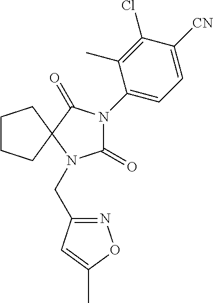 [SMILES: Cc1cc(CN2C(=O)N(c3ccc(C#N)c(Cl)c3C)C(=O)C23CCCC3)no1] | 6 + 1 (109 + 30) |
| 8.2 | 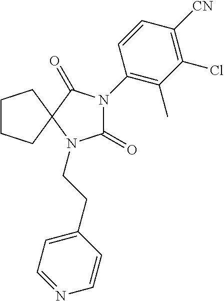 [SMILES: Cc1c(N2C(=O)N(CCc3ccncc3)C3(CCCC3)C2=O)ccc(C#N)c1Cl] | 202.5 (70) |
| 8.3 | 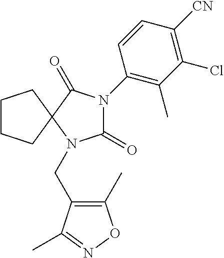 [SMILES: Cc1noc(C)c1CN1C(=O)N(c2ccc(C#N)c(Cl)c2C)C(=O)C12CCCC2] | 3/19* |
| 8.4 | 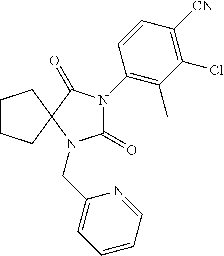 [SMILES: Cc1c(N2C(=O)N(Cc3ccccn3)C3(CCCC3)C2=O)ccc(C#N)c1Cl] | 48 + 13 (96 + 12) |
| 8.5 | 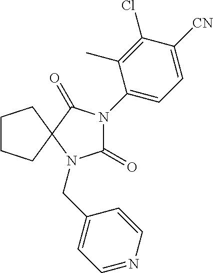 [SMILES: Cc1c(N2C(=O)N(Cc3ccncc3)C3(CCCC3)C2=O)ccc(C#N)c1Cl] | 30.7 + 8 |
| 8.6 | 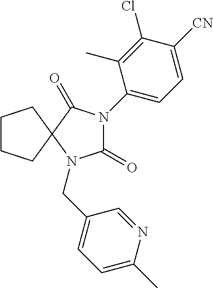 [SMILES: Cc1ccc(CN2C(=O)N(c3ccc(C#N)c(Cl)c3C)C(=O)C23CCCC3)cn1] | 11 + 8 |
| 8.7 | 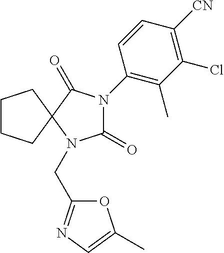 [SMILES: Cc1cnc(CN2C(=O)N(c3ccc(C#N)c(Cl)c3C)C(=O)C23CCCC3)o1] | 8.9 + 4.3 (112 + 2) |
| 8.8 | 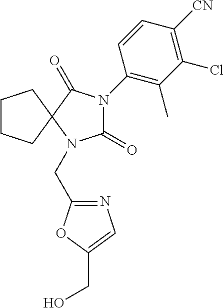 [SMILES: Cc1c(N2C(=O)N(Cc3ncc(CO)o3)C3(CCCC3)C2=O)ccc(C#N)c1Cl] | 26/70* |
| 8.9 | 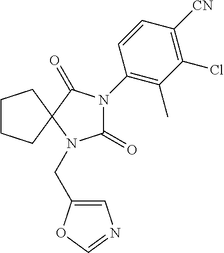 [SMILES: Cc1c(N2C(=O)N(Cc3cnco3)C3(CCCC3)C2=O)ccc(C#N)c1Cl] | 25 + 13 (87 + 2) |
| 8.10 | 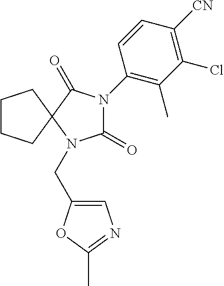 [SMILES: Cc1ncc(CN2C(=O)N(c3ccc(C#N)c(Cl)c3C)C(=O)C23CCCC3)o1] | 20/66* |
| 9.0 | 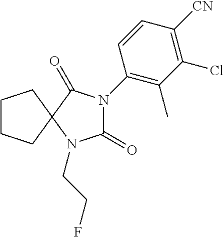 [SMILES: Cc1c(N2C(=O)N(CCF)C3(CCCC3)C2=O)ccc(C#N)c1Cl] | 45.4 + 20 (99 + 12) |
| 9.1 | 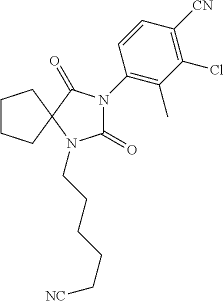 [SMILES: Cc1c(N2C(=O)N(CCCCCC#N)C3(CCCC3)C2=O)ccc(C#N)c1Cl] | 8.7 ± 2.1 (108 ± 7) |
| 9.2 | 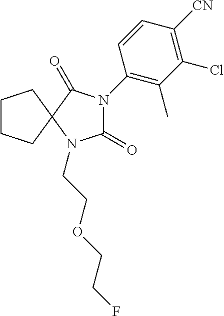 [SMILES: Cc1c(N2C(=O)N(CCOCCF)C3(CCCC3)C2=O)ccc(C#N)c1Cl] | 17.8 ± 6.6 (83 ± 17) |
| 9.3 | 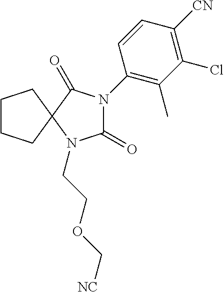 [SMILES: Cc1c(N2C(=O)N(CCOCC#N)C3(CCCC3)C2=O)ccc(C#N)c1Cl] | 19.9 ± 1.7 (91 ± 5.6) |
| 9.4 | 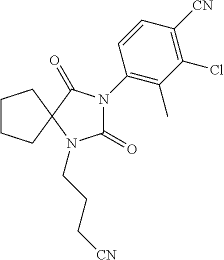 [SMILES: Cc1c(N2C(=O)N(CCCC#N)C3(CCCC3)C2=O)ccc(C#N)c1Cl] | 26.5 + 1 (90 + 14) |
| 9.5 | 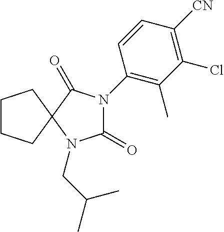 [SMILES: Cc1c(N2C(=O)N(CC(C)C)C3(CCCC3)C2=O)ccc(C#N)c1Cl] | 11/74* |
| 9.6 | 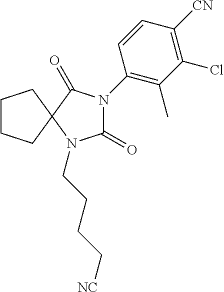 [SMILES: Cc1c(N2C(=O)N(CCCCC#N)C3(CCCC3)C2=O)ccc(C#N)c1Cl] | 45 + 3 (90 + 3) |
| 9.7 | 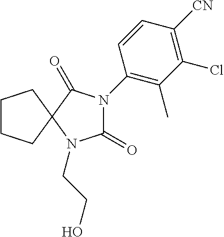 [SMILES: Cc1c(N2C(=O)N(CCO)C3(CCCC3)C2=O)ccc(C#N)c1Cl] | 198.5 (76) |
| 9.8 | 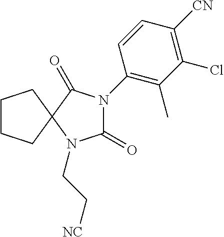 [SMILES: Cc1c(N2C(=O)N(CCC#N)C3(CCCC3)C2=O)ccc(C#N)c1Cl] | 169 ± 82 (85 ± 18) |
| 9.9 | 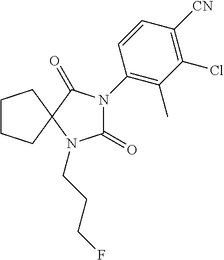 [SMILES: Cc1c(N2C(=O)N(CCCF)C3(CCCC3)C2=O)ccc(C#N)c1Cl] | 27/52* |
| 10.0 | 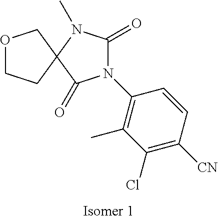 [SMILES: Cc1c(N2C(=O)N(C)C3(CCOC3)C2=O)ccc(C#N)c1Cl] | 198 ± 99 (60 ± 2) |
| 10.0 | 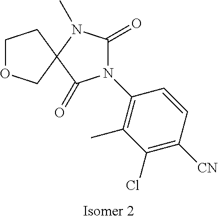 [SMILES: Cc1c(N2C(=O)N(C)C3(CCOC3)C2=O)ccc(C#N)c1Cl] | 93 ± 63 (65 ± 6) |
| 11.0 | 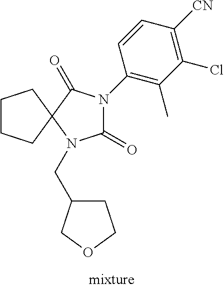 [SMILES: Cc1c(N2C(=O)N(CC3CCOC3)C3(CCCC3)C2=O)ccc(C#N)c1Cl] | 25/48* |
| 12.0 | 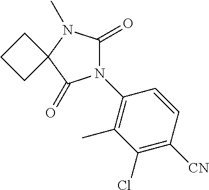 [SMILES: Cc1c(N2C(=O)N(C)C3(CCC3)C2=O)ccc(C#N)c1Cl] | 67/70* |

*% Biological activity (C2C12 cell) 100 nM/5 μM
** at 5 μM concentration, <10% activity

The compound 2-chloro-4-(2,4-dioxo-1-((tetrahydro-2H-pyran-4-yl)methyl)-1,3-diazaspiro[4.4]nonan-3-yl)-3-methylbenzonitrile shows 5% and 12% biological activity (C2C12 Cell) at 100 nM/5 μM respectively.

The compounds 2-chloro-4-(4-hydroxy-1-methyl-2-oxo-1,3-diazaspiro[4.4]nonan-3-yl)-3-methylbenzonitrile, 2-chloro-4-(4-methoxy-1-methyl-2-oxo-1,3-diazaspiro[4.4]nonan-3-yl)-3-methylbenzonitrile, 2-chloro-4-(6-hydroxy-2-oxo-1-oxa-3-azaspiro[4.4]nonan-3-yl)-3-methylbenzonitrile, 2-chloro-4-(6-fluoro-2-oxo-1-oxa-3-azaspiro[4.4]nonan-3-yl)-3-methylbenzonitrile, and 2-chloro-4-(6-fluoro-1-methyl-2-oxo-1,3-diazaspiro[4.4]nonan-3-yl)-3-methylbenzonitrile, 2-chloro-4-(2,4-dioxo-1-(2-(tetrahydro-2H-pyran-4-yl)ethyl)-1,3-diazaspiro[4.4]nonan-3-yl)-3-methylbenzonitrile, 2-chloro-3-methyl-4-(1-(2-morpholinoethyl)-2,4-dioxo-1,3-diazaspiro[4.4]nonan-3-yl)benzonitrile, 2-chloro-4-(1-(2-ethoxyethyl)-2,4-dioxo-1,3-diazaspiro[4.4]nonan-3-yl)-3-methylbenzonitrile, 2-chloro-4-(1-(2-isobutoxyethyl)-2,4-dioxo-1,3-diazaspiro[4.4]nonan-3-yl)-3-methylbenzonitrile, 2-chloro-4-(1-(2-isopropoxyethyl)-2,4-dioxo-1,3-diazaspiro[4.4]nonan-3-yl)-3-methylbenzonitrile exhibit efficacy in test 1 described above with EC₅₀ value >30 μM.

1. A compound of formula (I-1) in free form or in pharmaceutically acceptable salt form 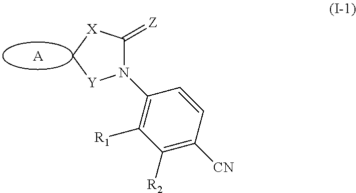
in which
X is O or N(R₈);
Y is CH₂, C(═NH), C(═O), C(═S) or C(H)(OR₉);
Z is O or S;
R₁ is C₁-C₃alkyl;
R₂ is halogen;
A is selected from:
a 4-membered saturated ring which contains zero or one O atom, which ring is unsubstituted or substituted once or twice with R_{A}; or
a 5-membered saturated or unsaturated non-aromatic ring which contains zero or one O atom, which ring is unsubstituted or substituted once or twice with R_{A};
R_{A} is, for each occurrence, independently selected from hydroxy, halogen, C₁-C₃alkyl, hydroxyC₁-C₃alkyl, or two R_{A} at the same carbon atom form an oxo group
R₈ is C1-C₆alkyl optionally substituted with cyano, hydroxy-C₁-C₆alkyl, haloC₁-C₆alkyl, C₁-C₆alkoxy-C₁-C₆alkyl wherein the alkoxy portion is optionally substituted with cyano or halogen or
R₈ is —(CH₂)ₙ—B;
n is 1 or 2;
B is a 5- to 6-membered aromatic or non-aromatic ring which comprises 0, 1, 2, 3, or 4 heteroatoms selected from N, O or S, which ring is unsubstituted or substituted once or twice with R_{B};
R_{B} is, for each occurrence, independently selected from halo, cyano, C₁-C₆alkyl; and
R₉ is hydrogen or C₁-C₃alkyl.

2. A compound of formula (I) in free form or in pharmaceutically acceptable salt form 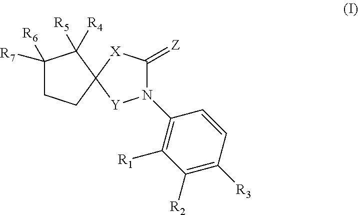
in which
X is O or N(R₈);
Y is CH₂, C(═NH), C(═O), C(═S) or C(H)(OR₉);
Z is O or S;
R₁ is C₁-C₃alkyl;
R₂ is halogen;
R₃ is cyano;
R₄ and R₅ are independently selected from hydrogen, hydroxy or halogen; or R₄ and R₅ together form an oxo group;
R₆ and R₇ are independently selected from hydrogen, hydroxy, or halogen; or R₆ and R₇ together form an oxo group; or
R₄ and R₆ together form a bond and R₅ and R₇ are each hydrogen;
R₈ is C₁-C₃alkyl, C₁-C₆alkoxy-C₁-C₃alkyl, hydroxy-C₁-C₃alkyl; and
R₉ is hydrogen or C₁-C₃alkyl.

3. A compound of claim 2 which is a compound of formula (Ia) in free form or in pharmaceutically acceptable salt form 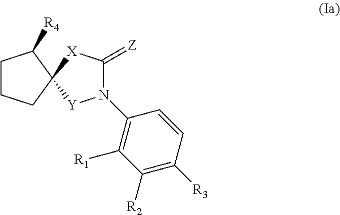
in which
X is O or N(R₈);
Y is CH₂, C(═NH), C(═O), C(═S) or C(H)(OR₉);
Z is O or S;
R₂ is halogen;
R₃ is cyano;
R₄ is hydrogen, hydroxy or halogen;
R₈ is C₁-C₃alkyl, C₁-C₆alkoxy-C₁-C₃alkyl, hydroxy-C₁-C₃alkyl; and
R₉ is hydrogen or C₁-C₃alkyl.

4. A compound of claim 2 which is a compound of formula (Ib) in free form or in pharmaceutically acceptable salt form 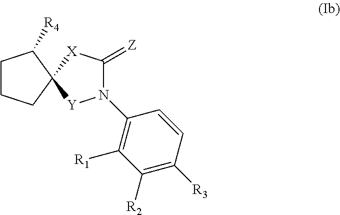
in which X is O or N(R₈);
Y is CH₂, C(═NH), C(═O), C(═S) or C(H)(OR₉);
Z is O or S;
R₂ is halogen;
R₃ is cyano;
R₄ is hydrogen, hydroxy or halogen;
R₈ is C₁-C₃alkyl, C₁-C₆alkoxy-C₁-C₃alkyl, hydroxy-C₁-C₃alkyl; and
R₉ is hydrogen or C₁-C₃alkyl.

5. A compound of claim 2 which is a compound of formula (Ic) in free form or in pharmaceutically acceptable salt form 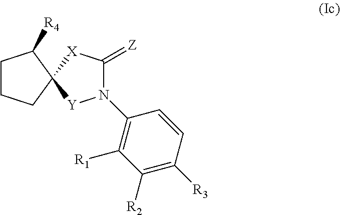
in which X is O or N(R₈);
Y is CH₂, C(═NH), C(═O), C(═S) or C(H)(OR₉);
Z is O or S;
R₂ is halogen;
R₃ is cyano;
R₄ is hydrogen, hydroxy or halogen;
R₈ is C₁-C₃alkyl, C₁-C₆alkoxy-C₁-C₃alkyl, hydroxy-C₁-C₃alkyl; and
R₉ is hydrogen or C₁-C₃alkyl.

6. A compound of claim 2 which is a compound of formula (Id) in free form or in pharmaceutically acceptable salt form 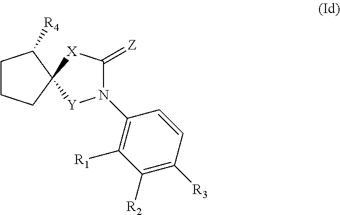
in which X is O or N(R₈);
Y is CH₂, C(═NH), C(═O), C(═S) or C(H)(OR₉);
Z is O or S;
R₂ is halogen;
R₃ is cyano;
R₄ is hydrogen, hydroxy or halogen;
R₈ is C₁-C₃alkyl, C₁-C₆alkoxy-C₁-C₃alkyl, hydroxy-C₁-C₃alkyl; and
R₉ is hydrogen or C₁-C₃alkyl.

7. A compound according to claim 1 in free form or in pharmaceutically acceptable salt form,
in which X is —N(CH₃), Y is —(C═O), and Z is O.

8. A compound according to claim 1 in free form or in pharmaceutically acceptable salt form,
in which R₁ is methyl and R₂ is chloro.

9. A compound according to claim 1 in free form or in pharmaceutically acceptable salt form which is selected from
2-chloro-4-(6-hydroxy-1-methyl-2,4-dioxo-1,3-diazaspiro[4.4]nonan-3-yl)-3-methylbenzonitrile;
2-chloro-3-methyl-4-(1-methyl-2,4,6-trioxo-1,3-diazaspiro[4.4]nonan-3-yl)benzonitrile;
2-chloro-4-(4,6-dihydroxy-1-methyl-2-oxo-1,3-diazaspiro[4.4]nonan-3-yl)-3-methylbenzonitrile;
2-chloro-4-(1-ethyl-6-hydroxy-2,4-dioxo-1,3-diazaspiro[4.4]nonan-3-yl)-3-methylbenzonitrile;
2-chloro-4-(6-fluoro-1-methyl-2,4-dioxo-1,3-diazaspiro[4.4]nonan-3-yl)-3-methylbenzonitrile;
2-chloro-3-methyl-4-(1-methyl-2,4-dioxo-1,3-diazaspiro[4.4]non-6-en-3-yl)benzonitrile;
2-chloro-4-(6-hydroxy-1-methyl-2-oxo-1,3-diazaspiro[4.4]nonan-3-yl)-3-methylbenzonitrile;
2-chloro-3-methyl-4-(1-methyl-2,4-dioxo-1,3-diazaspiro[4.4]nonan-3-yl)benzonitrile;
2-chloro-4-(1-(2-methoxyethyl)-2,4-dioxo-1,3-diazaspiro[4.4]nonan-3-yl)-3-methylbenzonitrile;
2-chloro-4-(1-ethyl-2,4-dioxo-1,3-diazaspiro[4.4]nonan-3-yl)-3-methylbenzonitrile;
2-chloro-4-(6-hydroxy-2,4-dioxo-1-oxa-3-azaspiro[4.4]nonan-3-yl)-3-methylbenzonitrile;
2-chloro-4-(6-hydroxy-4-methoxy-2-oxo-1-oxa-3-azaspiro[4.4]nonan-3-yl)-3-methylbenzonitrile;
2-chloro-3-methyl-4-(2-oxo-1-oxa-3-azaspiro[4.4]non-6-en-3-yl)benzonitrile;
2-chloro-4-(7-hydroxy-1-methyl-2,4-dioxo-1,3-diazaspiro[4.4]nonan-3-yl)-3-methylbenzonitrile;
2-chloro-4-(7-fluoro-1-methyl-2,4-dioxo-1,3-diazaspiro[4.4]nonan-3-yl)-3-methylbenzonitrile;
2-chloro-3-methyl-4-(1-methyl-2,4,7-trioxo-1,3-diazaspiro[4.4]nonan-3-yl)benzonitrile;
2-chloro-4-(7,7-difluoro-1-methyl-2,4-dioxo-1,3-diazaspiro[4.4]nonan-3-yl)-3-methylbenzonitrile;
2-chloro-4-(6-(hydroxymethyl)-1-methyl-2,4-dioxo-1,3-diazaspiro[4.4]nonan-3-yl)-3-methylbenzonitrile;
2-chloro-4-(1,6-dimethyl-2,4-dioxo-1,3-diazaspiro[4.4]nonan-3-yl)-3-methyl benzonitrile;
2-chloro-4-(4-imino-1-methyl-2-oxo-1,3-diazaspiro[4.4]nonan-3-yl)-3-methylbenzonitrile;
2-chloro-4-(1-(4-cyanobenzyl)-2,4-dioxo-1,3-diazaspiro[4.4]nonan-3-yl)-3-methylbenzonitrile;
2-chloro-3-methyl-4-(1-((5-methyl isoxazol-3-yl)methyl)-2,4-dioxo-1,3-diazaspiro[4.4]nonan-3-yl)benzonitrile;
2-chloro-4-(2,4-dioxo-1-(2-(pyridin-4-yl)ethyl)-1,3-diazaspiro[4.4]nonan-3-yl)-3-methylbenzonitrile;
2-chloro-4-(1-((3,5-dimethylisoxazol-4-yl)methyl)-2,4-dioxo-1,3-diazaspiro[4.4]nonan-3-yl)-3-methylbenzonitrile;
2-chloro-4-(2,4-dioxo-1-(pyridin-2-ylmethyl)-1,3-diazaspiro[4.4]nonan-3-yl)-3-methylbenzonitrile;
2-chloro-4-(2,4-dioxo-1-(pyridin-4-ylmethyl)-1,3-diazaspiro[4.4]nonan-3-yl)-3-methylbenzonitrile;
2-chloro-3-methyl-4-(1-((6-methyl pyridin-3-yl)methyl)-2,4-dioxo-1,3-diazaspiro[4.4]nonan-3-yl)benzonitrile;
2-chloro-3-methyl-4-(1-((5-methyloxazol-2-yl)methyl)-2,4-dioxo-1,3-diazaspiro[4.4]nonan-3-yl)benzonitrile;
2-chloro-4-(1-((5-(hydroxymethyl)oxazol-2-yl)methyl)-2,4-dioxo-1,3-diazaspiro[4.4]nonan-3-yl)-3-methylbenzonitrile;
2-chloro-3-methyl-4-(1-(oxazol-5-ylmethyl)-2,4-dioxo-1,3-diazaspiro[4.4]nonan-3-yl)benzonitrile;
2-chloro-3-methyl-4-(1-((2-methyloxazol-5-yl)methyl)-2,4-dioxo-1,3-diazaspiro[4.4]nonan-3-yl)benzonitrile;
2-chloro-4-(1-(2-fluoroethyl)-2,4-dioxo-1,3-diazaspiro[4.4]nonan-3-yl)-3-methyl benzonitrile;
2-chloro-4-(1-(5-cyanopentyl)-2,4-dioxo-1,3-diazaspiro[4.4]nonan-3-yl)-3-methylbenzonitrile;
2-chloro-4-(1-(2-(2-fluoroethoxy)ethyl)-2,4-dioxo-1,3-diazaspiro[4.4]nonan-3-yl)-3-methylbenzonitrile;
2-chloro-4-(1-(2-(cyanomethoxy)ethyl)-2,4-dioxo-1,3-diazaspiro[4.4]nonan-3-yl)-3-methylbenzonitrile;
2-chloro-4-(1-(3-cyanopropyl)-2,4-dioxo-1,3-diazaspiro[4.4]nonan-3-yl)-3-methyl benzonitrile;
2-chloro-4-(1-isobutyl-2,4-dioxo-1,3-diazaspiro[4.4]nonan-3-yl)-3-methylbenzonitrile;
2-chloro-4-(1-(4-cyanobutyl)-2,4-dioxo-1,3-diazaspiro[4.4]nonan-3-yl)-3-methylbenzonitrile;
2-chloro-4-(1-(2-hydroxyethyl)-2,4-dioxo-1,3-diazaspiro[4.4]nonan-3-yl)-3-methylbenzonitrile;
2-chloro-4-(1-(2-cyanoethyl)-2,4-dioxo-1,3-diazaspiro[4.4]nonan-3-yl)-3-methylbenzonitrile;
2-chloro-4-(1-(3-fluoropropyl)-2,4-dioxo-1,3-diazaspiro[4.4]nonan-3-yl)-3-methylbenzonitrile;
2-chloro-3-methyl-4-(1-methyl-2,4-dioxo-7-oxa-1,3-diazaspiro[4.4]nonan-3-yl)benzonitrile;
2-chloro-4-(2,4-dioxo-1-((tetrahydrofuran-3-yl)methyl)-1,3-diazaspiro[4.4]nonan-3-yl)-3-methylbenzonitrile; and
2-chloro-3-methyl-4-(5-methyl-6,8-dioxo-5,7-diazaspiro[3.4]octan-7-yl)benzonitrile.

10. A pharmaceutical composition comprising a therapeutically effective amount of a compound according to claim 1 in free form or in pharmaceutically acceptable salt form and one or more pharmaceutically acceptable carriers.

11. A combination comprising a therapeutically effective amount of a compound according to claim 1 in free form or in pharmaceutically acceptable salt form and one or more therapeutically active co-agents.

12. (canceled)

13. (canceled)

14. A method of treating a disorder or disease selected from muscle wasting diseases, osteoporosis, sarcopenia, frailty, and cancer cachexia, comprising administering to the subject a therapeutically effective amount of the compound according to claim 1 in free form or in pharmaceutically acceptable salt form.

15. A compound according to claim 2 in free form or in pharmaceutically acceptable salt form,
in which X is —N(CH₃), Y is —(C═O), and Z is O.

16. A compound according to claim 2 in free form or in pharmaceutically acceptable salt form,
in which R₁ is methyl and R₂ is chloro.

17. A pharmaceutical composition comprising a therapeutically effective amount of a compound according to claim 2 in free form or in pharmaceutically acceptable salt form and one or more pharmaceutically acceptable carriers.

18. A combination comprising a therapeutically effective amount of a compound according to claim 2 in free form or in pharmaceutically acceptable salt form and one or more therapeutically active co-agents.
